(19) United States
(12) Patent Application Publication
(10) Pub. No.: US 2008/0076166 A1
(43) Pub. Date: Mar. 27, 2008

(54) Method For Producing Polyunsaturated Fatty Acids In Transgenic Organisms

(75) Inventors: Petra Cirpus, Mannheim (DE); Jorg Bauer, Ludwigshafen (DE); Ernst Heinz, Hamburg (DE); Frederic Domergue, Hamburg (DE)

Correspondence Address: CONNOLLY BOVE LODGE & HUTZ, LLP, P O BOX 2207, WILMINGTON, DE, 19899, US

(73) Assignee: BASF PLANT SCIENCE GMBH, LUDWIGSHAFEN (DE)

(21) Appl. No.: 11/794,006

(22) PCT Filed: Dec. 21, 2005
(86) PCT No.: PCT/EP05/13803

§ 371 Date: Jun. 21, 2007

(30) Foreign Application Priority Data
Dec. 23, 2004 (DE) 10 2004 063 326.6

(51) Int. Cl. C12P 7/64; C07C 51/00; C07H 21/04; C07K 2/00; C12N 15/82

(52) U.S. Cl. 435/134; 435/320.1; 530/300; 536/23.6; 554/169

(57) ABSTRACT

The present invention relates to a process for the production of polyunsaturated fatty acids in an organism by introducing, into the organism, nucleic acids which encode polypeptides with Δ5-elongase, Δ6-desaturase, Δ5-desaturase, Δ4-desaturase, Δ12-desaturase and/or Δ6-elongase activity. These desaturases and elongases are advantageously derived from Ostreococcus. The invention furthermore relates to a process for the production of oils and/or triacylglycerides with an elevated content of long-chain polyunsaturated fatty acids. The invention furthermore relates to the nucleic acid sequences, nucleic acid constructs, vectors and organisms comprising the nucleic acid sequences according to the invention, to vectors comprising the nucleic acid sequences and/or the nucleic acid constructs and to transgenic organisms comprising the abovementioned nucleic acid sequences, nucleic acid constructs and/or vectors. A further part of the invention relates to oils, lipids and/or fatty acids produced by the process according to the invention and to their use. Moreover, the invention relates to unsaturated fatty acids and to triglycerides with an elevated content of unsaturated fatty acids and to their use.

The present invention relates to a process for the production of polyunsaturated fatty acids in an organism by introducing, into the organism, nucleic acids which encode polypeptides with Δ5-elongase, Δ6-desaturase, Δ5-desaturase, Δ4-desaturase, Δ12-desaturase and/or Δ6-elongase activity. These desaturases and elongases are advantageously derived from Ostreococcus. The invention furthermore relates to a process for the production of oils and/or triacylglycerides with an elevated content of long-chain polyunsaturated fatty acids.

The invention furthermore relates to the nucleic acid sequences, nucleic acid constructs, vectors and organisms comprising the nucleic acid sequences according to the invention, to vectors comprising the nucleic acid sequences and/or the nucleic acid constructs and to transgenic organisms comprising the abovementioned nucleic acid sequences, nucleic acid constructs and/or vectors.

A further part of the invention relates to oils, lipids and/or fatty acids produced by the process according to the invention and to their use. Moreover, the invention relates to unsaturated fatty acids and to triglycerides with an elevated content of unsaturated fatty acids and to their use.

Fatty acids and triacylglycerides have a multiplicity of applications in the food industry, in animal nutrition, in cosmetics and in the pharmacological sector. Depending on whether they are free saturated or unsaturated fatty acids or else triacylglycerides with an elevated content of saturated or unsaturated fatty acids, they are suitable for very different applications. Polyunsaturated fatty acids such as linoleic acid and linolenic acid are essential for mammals, since they cannot be produced by the latter. Polyunsaturated ω3-fatty acids and ω6-fatty acids are therefore an important constituent in animal and human nutrition.

Polyunsaturated long-chain ω3-fatty acids such as eicosapentaenoic acid (=EPA, C20:5^{Δ5,8,11,14,17}) or docosahexaenoic acid (=DHA, C22:6^{Δ4,7,10,13,16,19}) are important components in human nutrition owing to their various roles in health aspects, including the development of the child brain, the functionality of the eyes, the synthesis of hormones and other signal substances, and the prevention of cardiovascular disorders, cancer and diabetes (Poulos, A Lipids 30:1-14, 1995; Horrocks, L A and Yeo Y K Pharmacol Res 40:211-225, 1999). This is why there is a demand for the production of polyunsaturated long-chain fatty acids.

Owing to the currently customary composition of human food, an addition of polyunsaturated ω3-fatty acids, which are preferentially found in fish oils, to the food is particularly important. Thus, for example, polyunsaturated fatty acids such as docosahexaenoic acid (=DHA, C22:6^{Δ4,7,10,13,16,19}) or eicosapentaenoic acid (=EPA, C20:5^{Δ5,8,11,14,17}) are added to infant formula to improve the nutritional value. The unsaturated fatty acid DHA is said to have a positive effect on the development and maintenance of brain functions.

Hereinbelow, polyunsaturated fatty acids are referred to as PUFA, PUFAs, LCPUFA or LCPUFAs (poly unsaturated fatty acids, PUFA, long chain holy unsaturated fatty acids, LCPUFA).

The various fatty acids and triglycerides are mainly obtained from microorganisms such as Mortierella and Schizochytrium or from oil-producing plants such as soybean, oilseed rape, algae such as Crypthecodinium or Phaeodactylum and others, where they are obtained, as a rule, in the form of their triacylglycerides (=triglycerides=triglycerols). However, they can also be obtained from animals, such as, for example, fish. The free fatty acids are advantageously prepared by hydrolysis. Very long-chain polyunsaturated fatty acids such as DHA, EPA, arachidonic acid (=ARA, C20:4^{Δ5,8,11,14}), dihomo-γ-linolenic acid (C20:3^{Δ8,11,14}) or docosapentaenoic acid (DPA, C22:5^{Δ7,10,13,16,19}) are not synthesized in oil crops such as oilseed rape, soybean, sunflower or safflower. Conventional natural sources of these fatty acids are fish such as herring, salmon, sardine, redfish, eel, carp, trout, halibut, mackerel, zander or tuna, or algae.

Depending on the intended use, oils with saturated or unsaturated fatty acids are preferred. In human nutrition, for example, lipids with unsaturated fatty acids, specifically polyunsaturated fatty acids, are preferred. The polyunsaturated ω3-fatty acids are said to have a positive effect on the cholesterol level in the blood and thus on the possibility of preventing heart disease. The risk of heart disease, stroke or hypertension can be reduced markedly by adding these ω3-fatty acids to the food. Also, ω3-fatty acids have a positive effect on inflammatory, specifically on chronically inflammatory, processes in association with immunological diseases such as rheumatoid arthritis. They are therefore added to foodstuffs, specifically to dietetic foodstuffs, or are employed in medicaments. ω6-Fatty acids such as arachidonic acid tend to have a negative effect on these disorders in connection with these rheumatic diseases on account of our usual dietary intake.

ω3- and ω6-fatty acids are precursors of tissue hormones, known as eicosanoids, such as the prostaglandins, which are derived from dihomo-γ-linolenic acid, arachidonic acid and eicosapentaenoic acid, and of the thromboxanes and leukotrienes, which are derived from arachidonic acid and eicosapentaenoic acid. Eicosanoids (known as the PG₂ series) which are formed from ω6-fatty acids generally promote inflammatory reactions, while eicosanoids (known as the PG₃ series) from ω3-fatty acids have little or no proinflammatory effect.

Owing to the positive characteristics of the polyunsaturated fatty acids, there has been no lack of attempts in the past to make available genes which are involved in the synthesis of fatty acids or triglycerides for the production of oils in various organisms with a modified content of unsaturated fatty acids. Thus, WO 91/13972 and its US equivalent describes a Δ9-desaturase. WO 93/11245 claims a Δ15-desaturase and WO 94/11516 a Δ12-desaturase. Further desaturases are described, for example, in EP-A-0 550 162, WO 94/18337, WO 97/30582, WO 97/21340, WO 95/18222, EP-A-0 794 250, Stukey et al., J. Biol. Chem., 265, 1990: 20144-20149, Wada et al., Nature 347, 1990: 200-203 or Huang et al., Lipids 34, 1999: 649-659. However, the biochemical characterization of the various desaturases has been insufficient to date since the enzymes, being membrane-bound proteins, present great difficulty in their isolation and characterization (McKeon et al., Methods in Enzymol. 71, 1981: 12141-12147, Wang et al., Plant Physiol. Biochem., 26, 1988: 777-792). As a rule, membrane-bound desaturases are characterized by being introduced into a suitable organism which is subsequently analyzed for enzyme activity by analyzing the starting materials and the products. Δ6-Desaturases are described in WO 93/06712, U.S. Pat. No. 5,614,393, WO 96/21022, WO 00/21557 and WO 99/27111 and the application for the production in transgenic organisms is described in WO 98/46763, WO 98/46764 and WO 98/46765. In this context, the expression of various desaturases and the formation of polyunsaturated fatty acids is also described and claimed in WO 99/64616 or WO 98/46776. As regards the expression efficacy of desaturases and its effect on the formation of polyunsaturated fatty acids, it must be noted that the expression of a single desaturase as described to date has only resulted in low contents of unsaturated fatty acids/lipids such as, for example, γ-linolenic acid and stearidonic acid. Moreover, a mixture of ω3- and ω6-fatty acids was obtained, as a rule. Especially suitable microorganisms for the production of PUFAs are microalgae such as Phaeodactylum tricornutum, Porphiridium species, Thraustochytrium species, Schizochytrium species or Crypthecodinium species, ciliates such as Stylonychia or Colpidium, fungae such as Mortierella, Entomophthora or Mucor and/or mosses such as Physcomitrella, Ceratodon and Marchantia (R. Vazhappilly & F. Chen (1998) Botanica Marina 41: 553-558; K. Totani & K. Oba (1987) Lipids 22: 1060-1062; M. Akimoto et al. (1998) Appl. Biochemistry and Biotechnology 73: 269-278). Strain selection has resulted in the development of a number of mutant strains of the microorganisms in question which produce a series of desirable compounds including PUFAs. However, the mutation and selection of strains with an improved production of a particular molecule such as the polyunsaturated fatty acids is a time-consuming and difficult process. This is why recombinant methods as described above are preferred whenever possible.

However, only limited amounts of the desired polyunsaturated fatty acids such as DPA, EPA or ARA can be produced with the aid of the abovementioned microorganisms, and, depending on the microorganism used, these are generally obtained as fatty acid mixtures of, for example, EPA, DPA and ARA.

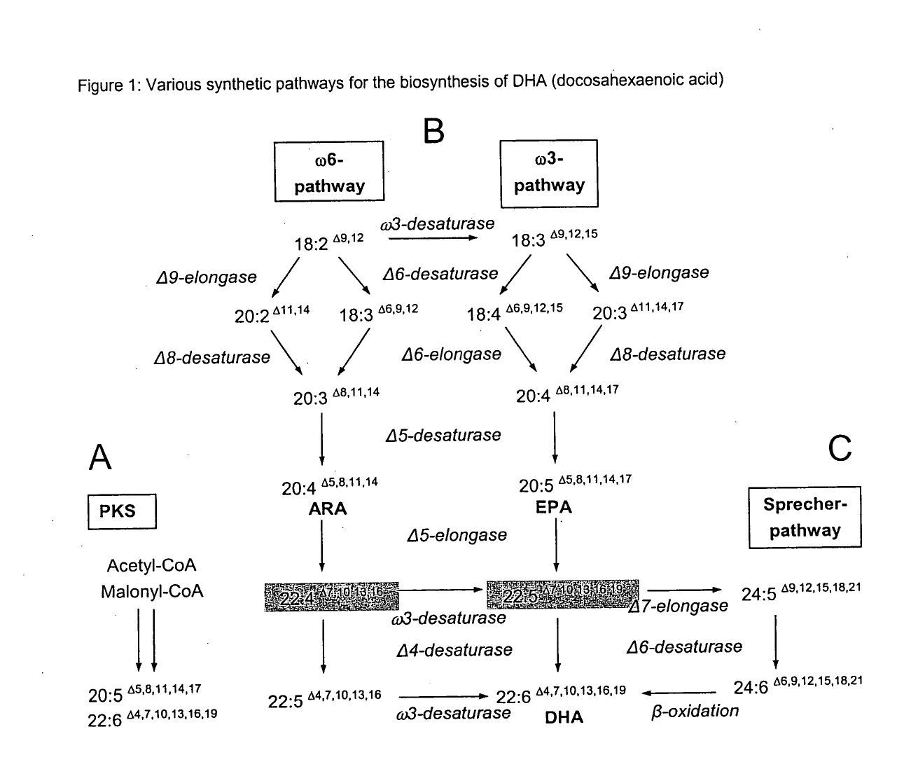

A variety of synthetic pathways is being discussed for the synthesis of arachidonic acid, eicosapentaenoic acid (EPA) and docosahexaenoic acid (DHA) (FIG. 1). Thus, EPA or DHA are produced in marine bacteria such as Vibrio sp. or Shewanella sp. via the polyketide pathway (Yu, R. et al. Lipids 35:1061-1064, 2000; Takeyama, H. et al. Microbiology 143:2725-2731, 1997).

An alternative strategy is the alternating activity of desaturases and elongases (Zank, T. K. et al. Plant Journal 31:255-268, 2002; Sakuradani, E. et al. Gene 238:445-453, 1999). A modification of the above-described pathway by Δ6-desaturase, Δ6-elongase, Δ5-desaturase, Δ5-elongase and Δ4-desaturase is the Sprecher pathway (Sprecher 2000, Biochim. Biophys. Acta 1486:219-231) in mammals. Instead of the Δ4-desaturation, a further elongation step is effected here to give C₂₄, followed by a further Δ6-desaturation and finally β-oxidation to give the C₂₂ chain length. Thus what is known as Sprecher pathway (see FIG. 1) is, however, not suitable for the production in plants and microorganisms since the regulatory mechanisms are not known. Depending on their desaturation pattern, the polyunsaturated fatty acids can be divided into two large classes, viz. ω6- or ω3-fatty acids, which differ with regard to their metabolic and functional activities (FIG. 1).

The starting material for the ω6-metabolic pathway is the fatty acid linoleic acid (18:2^{Δ9,12}) while the ω3-pathway proceeds via linolenic acid (18:3^{Δ9,12,15}). Linolenic acid is formed by the activity of an ω3-desaturase (Tocher et al. 1998, Prog. Lipid Res. 37, 73-117; Domergue et al. 2002, Eur. J. Biochem. 269, 4105-4113).

Mammals, and thus also humans, have no corresponding desaturase activity (Δ12- and ω3-desaturase) and must take up these fatty acids (essential fatty acids) via the food. Starting with these precursors, the physiologically important polyunsaturated fatty acids arachidonic acid (=ARA, 20:4^{Δ5,8,11,14}), an ω6-fatty acid and the two ω3-fatty acids eicosapentaenoic acid (=EPA, 20:5^{Δ5,8,11,14,17}) and docosahexaenoic acid (DHA, 22:6^{Δ4,7,10,13,17,19}) are synthesized via the sequence of desaturase and elongase reactions. The application of ω3-fatty acids shows the therapeutic activity described above in the treatment of cardiovascular diseases (Shimikawa 2001, World Rev. Nutr. Diet. 88, 100-108), inflammations (Calder 2002, Proc. Nutr. Soc. 61, 345-358) and arthritis (Cleland and James 2000, J. Rheumatol. 27, 2305-2307).

The elongation of fatty acids, by elongases, by 2 or 4 C atoms is of crucial importance for the production of C₂₀- and C₂₂-PUFAs, respectively. This process proceeds via 4 steps. The first step is the condensation of malonyl-CoA onto the fatty acid-acyl-CoA by ketoacyl-CoA synthase (KCS, hereinbelow referred to as elongase). This is followed by a reduction step (ketoacyl-CoA reductase, KCR), a dehydratation step (dehydratase) and a final reduction step (enoyl-CoA reductase). It has been postulated that the elongase activity affects the specificity and rate of the entire process (Millar and Kunst, 1997 Plant Journal 12:121-131).

There have been a large number of attempts in the past to obtain elongase genes. Millar and Kunst, 1997 (Plant Journal 12:121-131) and Millar et al. 1999, (Plant Cell 11:825-838) describe the characterization of plant elongases for the synthesis of monounsaturated long-chain fatty acids (C22:1) and for the synthesis of very long-chain fatty acids for the formation of waxes in plants (C₂₈-C₃₂). Descriptions regarding the synthesis of arachidonic acid and EPA are found, for example, in WO0159128, WO0012720, WO02077213 and WO0208401. The synthesis of polyunsaturated C₂₄-fatty acids is described, for example, in Tvrdik et al. 2000, JCB 149:707-717 or WO0244320.

No specific elongase has been described to date for the production of DHA (C22:6 n−3) in organisms which do not naturally produce this fatty acid. Only elongases which provide C₂₀- or C₂₄-fatty acids have been described to date. A Δ5-elongase activity has not been described to date.

Higher plants comprise polyunsaturated fatty acids such as linoleic acid (C18:2) and linolenic acid (C18:3). ARA, EPA and DHA are found not at all in the seed oil of higher plants, or only in miniscule amounts (E. Ucciani: Nouveau Dictionnaire des Huiles Végétales [New Dictionary of Vegetable Oils]. Technique & Documentation—Lavoisier, 1995. ISBN: 2-7430-0009-0). However, the production of LCPUFAs in higher plants, preferably in oil crops such as oilseed rape, linseed, sunflower and soybeans, would be advantageous since large amounts of high-quality LCPUFAs for the food industry, animal nutrition and pharmaceutical purposes might be obtained economically in this way. To this end, it is advantageous to introduce, into oil crops, genes which encode enzymes of the LCPUFA biosynthesis via recombinant methods and to express them therein. These genes encode for example Δ6-desaturases, Δ6-elongases, Δ5-desaturases or Δ4-desaturases. These genes can advantageously be isolated from microorganisms and lower plants which produce LCPUFAs and incorporate them in the membranes or triacylglycerides. Thus, it has already been possible to isolate Δ6-desaturase genes from the moss Physcomitrella patens and Δ6-elongase genes from P. patens and from the nematode C. elegans.

The first transgenic plants to comprise and express genes encoding LCPUFA biosynthesis enzymes and which produce LCPUFAs were described for the first time, for example, in DE 102 19 203 (process for the production of polyunsaturated fatty acids in plants). However, these plants produce LCPUFAs in amounts which require further optimization for processing the oils which are present in the plants.

To make possible the fortification of food and of feed with these polyunsaturated fatty acids, there is therefore a great need for a simple, inexpensive process for the production of these polyunsaturated fatty acids, specifically in eukaryotic systems.

It was therefore an object to provide further genes or enzymes which are suitable for the synthesis of LCPUFAs, specifically genes with Δ5-desaturase, Δ4-desaturase, Δ12-desaturase or Δ6-desaturase activity, for the production of polyunsaturated fatty acids. A further object of the present invention was the provision of genes or enzymes which make possible a shift from the ω6-fatty acids to the ω3-fatty acids. Another object was to develop a process for the production of polyunsaturated fatty acids in an organism, advantageously in a eukaryotic organism, preferably in a plant or a microorganism. This object was achieved by the process according to the invention for the production of compounds of the formula I 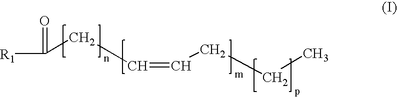
in transgenic organisms with a content of at least 1% by weight of these compounds based on the total lipid content of the transgenic organism, which comprises the following process steps:
a) introducing, into the organism, at least one nucleic acid sequence which encodes a Δ6-desaturase activity, and
b) introducing, into the organism, at least one nucleic acid sequence which encodes a Δ6-elongase activity, and
c) introducing, into the organism, at least one nucleic acid sequence which encodes a Δ5-desaturase activity, and
d) introducing, into the organism, at least one nucleic acid sequence which encodes a Δ5-elongase activity, and
e) introducing, into the organism, at least one nucleic acid sequence which encodes a Δ4-desaturase activity, and
   where the variables and substituents in formula I have the following meanings:
R¹=hydroxyl, coenzyme A (thioester), lysophosphatidylcholine, lysophosphatidylethanolamine, lysophosphatidylglycerol, lysodiphosphatidylglycerol, lysophosphatidylserine, lysophosphatidylinositol, sphingo base or a radical of the formula II 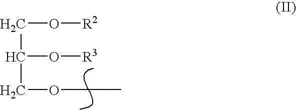
R²=hydrogen, lysophosphatidylcholine, lysophosphatidylethanolamine, lysophosphatidylglycerol, lysodiphosphatidylglycerol, lysophosphatidylserine, lysophosphatidylinositol or saturated or unsaturated C₂-C₂₄-alkylcarbonyl,
R³=hydrogen, saturated or unsaturated C₂-C₂₄-alkylcarbonyl, or R² and R³ independently of one another are a radical of the formula Ia: 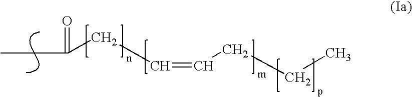
   in which

n=2, 3, 4, 5, 6, 7 or 9, m=2, 3, 4, 5 or 6 and p=0 or 3.

R¹ in the formula I is hydroxyl, coenzyme A (thioester), lysophosphatidylcholine, lysophosphatidylethanolamine, lysophosphatidylglycerol, lysodiphosphatidylglycerol, lysophosphatidylserine, lysophosphatidylinositol, sphingo base or a radical of the formula II

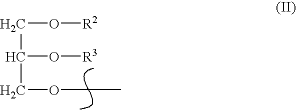

The abovementioned radicals of R¹ are always bonded to the compounds of the formula I in the form of their thioesters.

R² in the formula II is hydrogen, lysophosphatidylcholine, lysophosphatidylethanolamine, lysophosphatidylglycerol, lysodiphosphatidylglycerol, lysophosphatidylserine, lysophosphatidylinositol or saturated or unsaturated C₂-C₂₄-alkylcarbonyl.

Alkyl radicals which may be mentioned are substituted or unsubstituted, saturated or unsaturated C₂-C₂₄-alkylcarbonyl chains such as ethylcarbonyl, n-propylcarbonyl, n-butylcarbonyl, n-pentylcarbonyl, n-hexylcarbonyl, n-heptylcarbonyl, n-octylcarbonyl, n-nonylcarbonyl, n-decylcarbonyl, n-undecylcarbonyl, n-dodecylcarbonyl, n-tridecylcarbonyl, n-tetradecylcarbonyl, n-pentadecylcarbonyl, n-hexadecylcarbonyl, n-heptadecylcarbonyl, n-octadecylcarbonyl-, n-nonadecylcarbonyl, n-eicosylcarbonyl, n-docosanylcarbonyl- or n-tetracosanylcarbonyl, which comprise one or more double bonds. Saturated or unsaturated C₁₀-C₂₂-alkylcarbonyl radicals such as n-decylcarbonyl, n-undecylcarbonyl, n-dodecylcarbonyl, n-tridecylcarbonyl, n-tetradecylcarbonyl, n-pentadecylcarbonyl, n-hexadecylcarbonyl, n-heptadecylcarbonyl, n-octadecylcarbonyl, n-nonadecylcarbonyl, n-eicosylcarbonyl, n-docosanylcarbonyl or n-tetracosanylcarbonyl, which comprise one or more double bonds are preferred. Especially preferred are saturated and/or unsaturated C₁₀-C₂₂-alkylcarbonyl radicals such as C₁₀-alkylcarbonyl, C₁₁alkylcarbonyl, C₁₂-alkylcarbonyl, C₁₃-alkylcarbonyl, C14-alkylcarbonyl, C₁₆-alkylcarbonyl, C₁₈-alkylcarbonyl, C₂₀-alkylcarbonyl or C₂₂-alkylcarbonyl radicals which comprise one or more double bonds. Very especially preferred are saturated or unsaturated C₁₆-C₂₂-alkylcarbonyl radicals such as C₁₆-alkylcarbonyl, C₁₈-alkylcarbonyl, C₂₀-alkylcarbonyl or C₂₂-alkylcarbonyl radicals which comprise one or more double bonds. These advantageous radicals can comprise two, three, four, five or six double bonds. The especially advantageous radicals with 20 or 22 carbon atoms in the fatty acid chain comprise up to six double bonds, advantageously three, four, five or six double bonds, especially preferably five or six double bonds. All the abovementioned radicals are derived from the corresponding fatty acids.

R³ in the formula II is hydrogen, saturated or unsaturated C₂-C₂₄-alkylcarbonyl.

Alkyl radicals which may be mentioned are substituted or unsubstituted, saturated or unsaturated C₂-C₂₄-alkylcarbonyl chains such as ethylcarbonyl, n-propylcarbonyl, n-butylcarbonyl-, n-pentylcarbonyl, n-hexylcarbonyl, n-heptylcarbonyl, n-octylcarbonyl, n-nonylcarbonyl, n-decylcarbonyl, n-undecylcarbonyl, n-dodecylcarbonyl, n-tridecylcarbonyl, n-tetradecylcarbonyl, n-pentadecylcarbonyl, n-hexadecylcarbonyl, n-heptadecylcarbonyl, n-octadecylcarbonyl-, n-nonadecylcarbonyl, n-eicosylcarbonyl, n-docosanylcarbonyl- or n-tetracosanylcarbonyl, which comprise one or more double bonds. Saturated or unsaturated C₁₀-C₂₂-alkylcarbonyl radicals such as n-decylcarbonyl, n-undecylcarbonyl, n-dodecylcarbonyl, n-tridecylcarbonyl, n-tetradecylcarbonyl, n-pentadecylcarbonyl, n-hexadecylcarbonyl, n-heptadecylcarbonyl, n-octadecylcarbonyl, n-nonadecylcarbonyl, n-eicosylcarbonyl, n-docosanylcarbonyl or n-tetracosanylcarbonyl, which comprise one or more double bonds are preferred. Especially preferred are saturated and/or unsaturated C₁₀-C₂₂-alkylcarbonyl radicals such as C₁₀-alkylcarbonyl, C₁₁-alkylcarbonyl, C₁₂-alkylcarbonyl, C₁₃-alkylcarbonyl, C₁₄-alkylcarbonyl, C₁₆-alkylcarbonyl, C₁₈-alkylcarbonyl, C₂₀-alkylcarbonyl or C₂₂-alkylcarbonyl radicals which comprise one or more double bonds. Very especially preferred are saturated or unsaturated C₁₆-C₂₂-alkylcarbonyl radicals such as C₁₆-alkylcarbonyl, C₁₈-alkylcarbonyl, C₂₀-alkylcarbonyl or C₂₂-alkylcarbonyl radicals which comprise one or more double bonds. These advantageous radicals can comprise two, three, four, five or six double bonds. The especially advantageous radicals with 20 or 22 carbon atoms in the fatty acid chain comprise up to six double bonds, advantageously three, four, five or six double bonds, especially preferably five or six double bonds. All the abovementioned radicals are derived from the corresponding fatty acids.

The abovementioned radicals of R¹, R² and R³ can be substituted by hydroxyl and/or epoxy groups and/or can comprise triple bonds.

The polyunsaturated fatty acids produced in the process according to the invention advantageously comprise at least two, advantageously three, four, five or six, double bonds. The fatty acids especially advantageously comprise four, five or six double bonds. Fatty acids produced in the process advantageously have 18, 20 or 22 C atoms in the fatty acid chain; the fatty acids preferably comprise 20 or 22 carbon atoms in the fatty acid chain. Saturated fatty acids are advantageously reacted to a minor degree, or not at all, with the nucleic acids used in the process. To a minor degree is to be understood as meaning that the saturated fatty acids are reacted with less than 5% of the activity, advantageously less than 3%, especially advantageously with less than 2%, very especially preferably with less than 1, 0.5, 0.25 or 0.125% in comparison with polyunsaturated fatty acids. These fatty acids which have been produced can be produced in the process as a single product or be present in a fatty acid mixture.

The nucleic acid sequences used in the process according to the invention are isolated nucleic acid sequences which encode polypeptides with Δ6-desaturase, Δ6-elongase, Δ5-desaturase, Δ5-elongase and/or Δ4-desaturase activity.

Nucleic acid sequences which are advantageously used in the process according to the invention are those which encode polypeptides with Δ6-desaturase, Δ6-elongase, Δ5-desaturase, Δ5-elongase or Δ4-desaturase activity, selected from the group consisting of:
a) a nucleic acid sequence with the sequence shown in SEQ ID NO:1, SEQ ID NO:3, SEQ ID NO:5, SEQ ID NO:7, SEQ ID NO:9, SEQ ID NO:11 or SEQ ID NO:13, or
b) nucleic acid sequences which, as the result of the degeneracy of the genetic code, can be derived from the amino acid sequences shown in SEQ ID NO:2, SEQ ID NO:4, SEQ ID NO:6, SEQ ID NO:8, SEQ ID NO:10, SEQ ID NO:12 or SEQ ID NO:14, or
c) derivatives of the nucleic acid sequence shown in SEQ ID NO:1, SEQ ID NO:3, SEQ ID NO:5, SEQ ID NO:7, SEQ ID NO:9, SEQ ID NO:11 or SEQ ID NO:13 which encode polypeptides with at least 40% identity at the amino acid level with SEQ ID NO:2, SEQ ID NO:4, SEQ ID NO:6, SEQ ID NO:8, SEQ ID NO:10, SEQ ID NO:12 or SEQ ID NO:14 and which have Δ6-desaturase, Δ6-elongase, Δ5-desaturase, Δ5-elongase or Δ4-desaturase activity.

The substituents R² or R³ in the formulae I and II are advantageously and independently of one another saturated or unsaturated C₁₈-C₂₂-alkylcarbonyl, especially advantageously they are, independently of one another, unsaturated C₁₈-, C₂₀- or C₂₂-alkylcarbonyl with at least two double bonds.

In a further preferred embodiment, the process comprises the additional introduction, into the organism, of a nucleic acid sequence which encodes polypeptides with Δ12-desaturase activity, selected from the group consisting of:
a) a nucleic acid sequence with the sequence shown in SEQ ID NO:15, or
b) nucleic acid sequences which, as the result of the degeneracy of the genetic code, can be derived from the amino acid sequence shown in SEQ ID NO:16, or
c) derivatives of the nucleic acid sequence shown in SEQ ID NO:15 which encode polypeptides with at least 50% identity at the amino acid level with SEQ ID NO:16 and which have Δ12-desaturase activity.

These abovementioned Δ12-desaturase sequences can be used together with the nucleic acid sequences used in the process and which encode Δ6-desaturases, Δ6-elongases, Δ5-desaturases, Δ5-elongases and/or Δ4-desaturases, alone or in combination with the ω3-desaturase sequences.

Table 1 shows the nucleic acid sequences, the organism of origin and the SEQ ID NO.

| No. | Organism | Activity | Sequence number |
|---|---|---|---|
| 1. | Ostreococcus tauri | Δ5-elongase | SEQ ID NO: 1 |
| 2. | Ostreococcus tauri | Δ5-elongase | SEQ ID NO: 3 |
| 3. | Ostreococcus tauri | Δ6-elongase | SEQ ID NO: 5 |
| 4. | Ostreococcus tauri | Δ4-desaturase | SEQ ID NO: 7 |
| 5. | Ostreococcus tauri | Δ5-desaturase | SEQ ID NO: 9 |
| 6. | Ostreococcus tauri | Δ5-desaturase | SEQ ID NO: 11 |
| 7. | Ostreococcus tauri | Δ6-desaturase | SEQ ID NO: 13 |
| 8. | Ostreococcus tauri | Δ12-desaturase | SEQ ID NO: 15 |

The polyunsaturated fatty acids produced in the process are advantageously bound in membrane lipids and/or triacylglycerides, but may also occur in the organisms as free fatty acids or else bound in the form of other fatty acid esters. In this context, they may be present as “pure products” or else advantageously in the form of mixtures of various fatty acids or mixtures of different glycerides. The various fatty acids which are bound in the triacylglycerides can be derived from short-chain fatty acids with 4 to 6 C atoms, medium-chain fatty acids with 8 to 12 C atoms or long-chain fatty acids with 14 to 24 C atoms; preferred are long-chain fatty acids, more preferably long-chain polyunsaturated fatty acids with 18, 20 and/or 22 C atoms.

The process according to the invention advantageously yields fatty acid esters with polyunsaturated C₁₈-, C₂₀- and/or C₂₂-fatty acid molecules with at least two double bonds in the fatty acid ester, advantageously with at least three, four, five or six double bonds in the fatty acid ester, especially advantageously with at least five or six double bonds in the fatty acid ester and advantageously leads to the synthesis of linoleic acid (=LA, C18:2^{Δ9,12}), γ-linolenic acid (=GLA, C18:3^{Δ6,9,12}), stearidonic acid (=SDA, C18:4^{Δ6,9,12,15}), dihomo-γ-linolenic acid (=DGLA, 20:3^{Δ8,11,14}), ω3-eicosatetraenoic acid (=ETA, C20:4^{Δ5,8,11,14}), arachidonic acid (ARA, C20:4^{Δ5,8,11,14}), eicosapentaenoic acid (EPA, C20:5^{Δ5,8,11,14,17}), ω6-docosapentaenoic acid (C22:5^{Δ4,7,10,13,16}), ω6-docosatetraenoic acid (C22:4^{Δ7,10,13,16}), ω3-docosapentaenoic acid (=DPA, C22:5^{Δ7,10,13,16,19}), docosahexaenoic acid (=DHA, C22:6^{Δ4,7,10,13,16,19}) or mixtures of these, preferably ARA, EPA and/or DHA. ω3-Fatty acids such as EPA and/or DHA are very especially preferably produced.

The fatty acid esters with polyunsaturated C₁₈-, C₂₀- and/or C₂₂-fatty acid molecules can be isolated in the form of an oil or lipid, for example in the form of compounds such as sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, monoacylglycerides, diacylglycerides, triacylglycerides or other fatty acid esters such as the acetyl-coenzyme A esters which comprise the polyunsaturated fatty acids with at least two, three, four, five or six, preferably five or six double bonds, from the organisms which have been used for the preparation of the fatty acid esters; advantageously, they are isolated in the form of their diacylglycerides, triacylglycerides and/or in the form of phosphatidylcholine, especially preferably in the form of the triacylglycerides. In addition to these esters, the polyunsaturated fatty acids are also present in the organisms, advantageously the plants, as free fatty acids or bound in other compounds. As a rule, the various abovementioned compounds (fatty acid esters and free fatty acids) are present in the organisms with an approximate distribution of 80 to 90% by weight of triglycerides, 2 to 5% by weight of diglycerides, 5 to 10% by weight of monoglycerides, 1 to 5% by weight of free fatty acids, 2 to 8% by weight of phospholipids, the total of the various compounds amounting to 100% by weight.

The process according to the invention yields the LCPUFAs produced in a content of at least 3% by weight, advantageously at least 5% by weight, preferably at least 8% by weight, especially preferably at least 10% by weight, most preferably at least 15% by weight, based on the total fatty acids in the transgenic organisms, advantageously in a transgenic plant. In this context, it is advantageous to convert C₁₈- and/or C₂₀-fatty acids which are present in the host organisms to at least 10%, advantageously to at least 20%, especially advantageously to at least 30%, most advantageously to at least 40% to give the corresponding products such as DPA or DHA, to mention just two examples. The fatty acids are advantageously produced in bound form. These unsaturated fatty acids can, with the aid of the nucleic acids used in the process according to the invention, be positioned at the sn1, sn2 and/or sn3 position of the advantageously produced triglycerides. Since a plurality of reaction steps are performed by the starting compounds linoleic acid (C18:2) and linolenic acid (C18:3) in the process according to the invention, the end products of the process such as, for example, arachidonic acid (ARA), eicosapentaenoic acid (EPA), ω6-docosapentaenoic acid or DHA are not obtained as absolutely pure products; minor traces of the precursors are always present in the end product. If, for example, both linoleic acid and linolenic acid are present in the starting organism and the starting plant, the end products such as ARA, EPA or DHA are present as mixtures. The precursors should advantageously not amount to more than 20% by weight, preferably not to more than 15% by weight, especially preferably not to more than 10% by weight, most preferably not to more than 5% by weight, based on the amount of the end product in question. Advantageously, only ARA, EPA or only DHA, bound or as free acids, are produced as end products in a transgenic plant into the process according to the invention. If the compounds ARA, EPA and DHA are produced simultaneously, they are advantageously produced in a ratio of at least 1:1:2 (EPA:ARA:DHA), advantageously of at least 1:1:3, preferably 1:1:4, especially preferably 1:1:5.

Fatty acid esters or fatty acid mixtures produced by the process according to the invention advantageously comprise 6 to 15% of palmitic acid, 1 to 6% of stearic acid, 7-85% of oleic acid, 0.5 to 8% of vaccenic acid, 0.1 to 1% of arachic acid, 7 to 25% of saturated fatty acids, 8 to 85% of monounsaturated fatty acids and 60 to 85% of polyunsaturated fatty acids, in each case based on 100% and on the total fatty acid content of the organisms. Advantageous polyunsaturated fatty acids which are present in the fatty acid esters or fatty acid mixtures are preferably at least 0.1, 0.2, 0.3, 0.4, 0.5, 0.6, 0.7, 0.8, 0.9 or 1% of arachidonic acid, based on the total fatty acid content. Moreover, the fatty acid esters or fatty acid mixtures which have been produced by the process of the invention advantageously comprise fatty acids selected from the group of the fatty acids erucic acid (13-docosaenoic acid), sterculic acid (9,10-methyleneoctadec-9-enoic acid), malvalic acid (8,9-methyleneheptadec-8-enoic acid), chaulmoogric acid (cyclopentenedodecanoic acid), furan fatty acid (9,12-epoxyoctadeca-9,11-dienoic acid), vernolic acid (9,10-epoxyoctadec-12-enoic acid), tariric acid (6-octadecynoic acid), 6-nonadecynoic acid, santalbic acid (t11-octadecen-9-ynoic acid), 6,9-octadecenynoic acid, pyrulic acid (t10-heptadecen-8-ynoic acid), crepenynic acid (9-octadecen-12-ynoic acid), 13,14-dihydrooropheic acid, octadecen-13-ene-9,11-diynoic acid, petroselenic acid (cis-6-octadecenoic acid), 9c,12t-octadecadienoic acid, calendulic acid (8t10t12c-octadecatrienoic acid), catalpic acid (9t11t13c-octadecatrienoic acid), eleostearic acid (9c11t13t-octadecatrienoic acid), jacaric acid (8c10t12c-octadecatrienoic acid), punicic acid (9c11t13c-octadecatrienoic acid), parinaric acid (9c11t13t15c-octadecatetraenoic acid), pinolenic acid (all-cis-5,9,12-octadecatrienoic acid), laballenic acid (5,6-octadecadienallenic acid), ricinoleic acid (12-hydroxyoleic acid) and/or coriolic acid (13-hydroxy-9c,11t-octadecadienoic acid). The abovementioned fatty acids are, as a rule, advantageously only found in traces in the fatty acid esters or fatty acid mixtures produced by the process according to the invention, that is to say that, based on the total fatty acids, they occur to less than 30%, preferably to less than 25%, 24%, 23%, 22% or 21%, especially preferably to less than 20%, 0.15%, 10%, 9%, 8%, 7%, 6% or 5%, very especially preferably to less than 4%, 3%, 2% or 1%. The fatty acid esters or fatty acid mixtures produced by the process according to the invention advantageously comprise less than 0.1%, based on the total fatty acids, or no butyric acid, no cholesterol, no clupanodonic acid (=docosapentaenoic acid, C22:5^{Δ4,8,12,15,21}) and no nisinic acid (tetracosahexaenoic acid, C23:6^{Δ3,8,12,15,18,21}).

Owing to the nucleic acid sequences of the invention, or the nucleic acid sequences used in the process according to the invention, an increase in the yield of polyunsaturated fatty acids of at least 50%, advantageously of at least 80%, especially advantageously of at least 100%, very especially advantageously of at least 150%, in comparison with the nontransgenic starting organism, for example a yeast, an alga, a fungus or a plant such as Arabidopsis or linseed can be obtained when the fatty acids are detected by GC analysis (see examples).

Chemically pure polyunsaturated fatty acids or fatty acid compositions can also be synthesized by the processes described above. To this end, the fatty acids or the fatty acid compositions are isolated from the organism, such as the microorganisms or the plants or the culture medium in or on which the organisms have been grown, or from the organism and the culture medium, in the known manner, for example via extraction, distillation, crystallization, chromatography or a combination of these methods. These chemically pure fatty acids or fatty acid compositions are advantageous for applications in the food industry sector, the cosmetic sector and especially the pharmacological industry sector.

Suitable organisms for the production in the process according to the invention are, in principle, any organisms such as microorganisms, nonhuman animals or plants. Plants which are suitable are, in principle, all those plants which are capable of synthesizing fatty acids, such as all dicotyledonous or monocotyledonous plants, algae or mosses. Advantageous plants are selected from the group of the plant families Adelotheciaceae, Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Crypthecodiniaceae, Cucurbitaceae, Ditrichaceae, Elaeagnaceae, Ericaceae, Euphorbiaceae, Fabaceae, Geraniaceae, Gramineae, Juglandaceae, Lauraceae, Leguminosae, Linaceae, Prasinophyceae or vegetable plants or ornamentals such as Tagetes.

Examples which may be mentioned are the following plants selected from the group consisting of: Adelotheciaceae such as the genera Physcomitrella, for example the genus and species Physcomitrella patens, Anacardiaceae such as the genera Pistacia, Mangifera, Anacardium, for example the genus and species Pistacia vera [pistachio], Mangifer indica [mango] or Anacardium occidentale [cashew], Asteraceae, such as the genera Calendula, Carthamus, Centaurea, Cichorium, Cynara, Helianthus, Lactuca, Locusta, Tagetes, Valeriana, for example the genus and species Calendula officinalis [common marigold], Carthamus tinctorius [safflower], Centaurea cyanus [cornflower], Cichorium intybus [chicory], Cynara scolymus [artichoke], Helianthus annus [sunflower], Lactuca sativa, Lactuca crispa, Lactuca esculenta, Lactuca scariola L. ssp. sativa, Lactuca scariola L. var. integrata, Lactuca scariolaL. var. integrifolia, Lactuca sativa subsp. romana, Locusta communis, Valeriana locusta [salad vegetables], Tagetes lucida, Tagetes erecta or Tagetes tenuifolia [african or french marigold], Apiaceae, such as the genus Daucus, for example the genus and species Daucus carota [carrot], Betulaceae, such as the genus Corylus, for example the genera and species Corylus avellana or Corylus colurna [hazelnut], Boraginaceae, such as the genus Borago, for example the genus and species Borago officinalis [borage], Brassicaceae, such as the genera Brassica, Camelina, Melanosinapis, Sinapis, Arabadopsis, for example the genera and species Brassica napus, Brassica rapa ssp. [oilseed rape], Sinapis arvensis Brassica juncea, Brassica juncea var. juncea, Brassica juncea var. crispifolia, Brassica juncea var. foliosa, Brassica nigra, Brassica sinapioides, Camelina sativa, Melanosinapis communis [mustard], Brassica oleracea [fodder beet] or Arabidopsis thaliana, Bromeliaceae, such as the genera Anana, Bromelia (pineapple), for example the genera and species Anana comosus, Ananas ananas or Bromelia comosa [pineapple], Caricaceae, such as the genus Carica, such as the genus and species Carica papaya [pawpaw], Cannabaceae, such as the genus Cannabis, such as the genus and species Cannabis sative [hemp], Convolvulaceae, such as the genera Ipomea, Convolvulus, for example the genera and species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, batate], Chenopodiaceae, such as the genus Beta, such as the genera and species Beta vulgaris, Beta vulgaris var. altissima, Beta vulgaris var. vulgaris, Beta maritima, Beta vulgaris var. perennis, Beta vulgaris var. conditiva or Beta vulgaris var. esculenta [sugarbeet], Crypthecodiniaceae, such as the genus Crypthecodinium, for example the genus and species Cryptecodinium cohnii, Cucurbitaceae, such as the genus Cucurbita, for example the genera and species Cucurbita maxima, Cucurbita mixta, Cucurbita pepo or Cucurbita moschata [pumpkin/squash], Cymbellaceae, such as the genera Amphora, Cymbella, Okedenia, Phaeodactylum, Reimeria, for example the genus and species Phaeodactylum tricomutum, Ditrichaceae, such as the genera Ditrichaceae, Astomiopsis, Ceratodon, Chrysoblastella, Ditrichum, Distichium, Eccremidium, Lophidion, Philibertiella, Pleuridium, Saelania, Trichodon, Skottsbergia, for example the genera and species Ceratodon antarcticus, Ceratodon columbiae, Ceratodon heterophyllus, Ceratodon purpurascens, Ceratodon purpureus, Ceratodon purpureus ssp. convolutus, Ceratodon purpureus ssp. stenocarpus, Ceratodon purpureus var. rotundifolius, Ceratodon ratodon, Ceratodon stenocarpus, Chrysoblastella chilensis, Ditrichum ambiguum, Ditrichum brevisetum, Ditrichum crispatissimum, Ditrichum difficile, Ditrichum falcifolium, Ditrichum flexicaule, Ditrichum giganteum, Ditrichum heteromallum, Ditrichum lineare, Ditrichum montanum, Ditrichum montanum, Ditrichum pallidum, Ditrichum punctulatum, Ditrichum pusillum, Ditrichum pusillum var. tortile, Ditrichum rhynchostegium, Ditrichum schimperi, Ditrichum tortile, Distichium capillaceum, Distichium hagenii, Distichium inclinatum, Distichium macounii, Eccremidium floridanum, Eccremidium whiteleggei, Lophidion strictus, Pleuridium acuminatum, Pleuridium alternifolium, Pleuridium holdridgei, Pleuridium mexicanum, Pleuridium ravenelii, Pleuridium subulatum, Saelania glaucescens, Trichodon borealis, Trichodon cylindricus or Trichodon cylindricus var. oblongus, Elaeagnaceae, such as the genus Elaeagnus, for example the genus and species Olea europaea [olive], Ericaceae, such as the genus Kalmia, for example the genera and species Kalmia latifolia, Kalmia angustifolia, Kalmia microphylia, Kalmia polifolia, Kalmia occidentalis, Cistus chamaerhodendros or Kalmia lucida [mountain laurel], Euphorbiaceae, such as the genera Manihot, Janipha, Jatropha, Ricinus, for example the genera and species Manihot utilissima, Janipha manihot, Jatropha manihot, Manihot aipil, Manihot dulcis, Manihot manihot, Manihot melanobasis, Manihot esculenta [cassaya] or Ricinus communis [castor-oil plant], Fabaceae, such as the genera Pisum, Albizia, Cathormion, Feuillea, Inga, Pithecolobium, Acacia, Mimosa, Medicajo, Glycine, Dolichos, Phaseolus, soybean, for example the genera and species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbeck, Feuilleea lebbeck, Mimosa lebbeck, Mimosa speciosa, silk tree Medicago sativa, Medicago falcata, Medicago varia [alfalfa] Glycine max Dolichos soja, Glycine gracilis, Glycine hispida, Phaseolus max, Soja hispida or Soja max [soybean], Funariaceae, such as the genera Aphanorrhegma, Entosthodon, Funaria, Physcomitrella, Physcomitrium, for example the genera and species Aphanorrhegma serratum, Entosthodon attenuatus, Entosthodon bolanderi, Entosthodon bonplandii, Entosthodon californicus, Entosthodon drummondii, Entosthodon jamesonii, Entosthodon leibergii, Entosthodon neoscoticus, Entosthodon rubrisetus, Entosthodon spathulifolius, Entosthodon tucsoni, Funaria americana, Funaria bolanderi, Funaria calcarea, Funaria californica, Funaria calvescens, Funaria convoluta, Funaria flavicans, Funaria groutiana, Funaria hygrometrica, Funaria hygrometrica var. arctica, Funaria hygrometrica var. calvescens, Funaria hygrometrica var. convoluta, Funaria hygrometrica var. muralis, Funaria hygrometrica var. utahensis, Funaria microstoma, Funaria microstoma var. obtusifolia, Funaria muhlenbergii, Funaria orcuttii, Funaria plano-convexa, Funaria polaris, Funaria ravenelii, Funaria rubriseta, Funaria serrata, Funaria sonorae, Funaria sublimbatus, Funaria tucsoni, Physcomitrella californica, Physcomitrella patens, Physcomitrella readeri, Physcomitrium australe, Physcomitrium californicum, Physcomitrium collenchymatum, Physcomitrium coloradense, Physcomitrium cupuliferum, Physcomitrium drummondii, Physcomitrium eurystomum, Physcomitrium flexifolium, Physcomitrium hookeri, Physcomitrium hookeri var. serratum, Physcomitrium immersum, Physcomitrium kellermanii, Physcomitrium megalocarpum, Physcomitrium pyriforme, Physcomitrium pyriforme var. serratum, Physcomitrium rufipes, Physcomitrium sandbergii, Physcomitrium subsphaericum, Physcomitrium washingtoniense, Geraniaceae, such as the genera Pelargonium, Cocos, Oleum, for example the genera and species Cocos nucifera, Pelargonium grossularioides or Oleum cocois [coconut], Gramineae, such as the genus Saccharum, for example the genus and species Saccharum officinarum, Juglandaceae, such as the genera Juglans, Wallia, for example the genera and species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut], Lauraceae, such as the genera Persea, Laurus, for example the genera and species Laurus nobilis [bay], Persea americana, Persea gratissima or Persea persea [avocado], Leguminosae, such as the genus Arachis, for example the genus and species Arachis hypogaea [peanut], Linaceae, such as the genera Linum, Adenolinum, for example the genera and species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. Iewisii, Linum pratense or Linum trigynum [linseed], Lythrarieae, such as the genus Punica, for example the genus and species Punica granatum [pomegranate], Malvaceae, such as the genus Gossypium, for example the genera and species Gossypium hirsutum, Gossypium arboreum, Gossypium barbadense, Gossypium herbaceum or Gossypium thurberi [cotton], Marchantiaceae, such as the genus Marchantia, for example the genera and species Marchantia berteroana, Marchantia foliacea, Marchantia macropora, Musaceae, such as the genus Musa, for example the genera and species Musa nana, Musa acuminata, Musa paradisiaca, Musa spp. [banana], Onagraceae, such as the genera Camissonia, Oenothera, for example the genera and species Oenothera biennis or Camissonia brevipes [evening primrose], Palmae, such as the genus Elaeis, for example the genus and species Elaeis guineensis [oil palm], Papaveraceae, such as, for example, the genus Papaver, for example the genera and species Papaver orientale, Papaver rhoeas, Papaver dubium [poppy], Pedaliaceae, such as the genus Sesamum, for example the genus and species Sesamum indicum [sesame], Piperaceae, such as the genera Piper, Artanthe, Peperomia, Steffensia, for example the genera and species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata [cayenne pepper], Poaceae, such as the genera Hordeum, Secale, Avena, Sorghum, Andropogon, Holcus, Panicum, Oryza, Zea (maize), Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum [barley], Secale cereale [rye], Avena sativa, Avena fatua, Avena byzantina, Avena fatua var. sativa, Avena hybrida [oats], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cemuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum [millet], Oryza sativa, Oryza latifolia [rice], Zea mays [maize] Triticum aestivum, Triticum durum, Triticum turgidum, Triticum hybemum, Triticum macha, Triticum sativum or Triticum vulgare [wheat], Porphyridiaceae, such as the genera Chroothece, Flintielia, Petrovanella, Porphyridium, Rhodella, Rhodosorus, Vanhoeffenia, for example the genus and species Porphyridium cruentum, Proteaceae, such as the genus Macadamia, for example the genus and species Macadamia intergrifolia [macadamia], Prasinophyceae, such as the genera Nephroselmis, Prasinococcus, Scherffelia, Tetraselmis, Mantoniella, Ostreococcus, for example the genera and species Nephroselmis olivacea, Prasinococcus capsulatus, Scherffelia dubia, Tetraselmis chui, Tetraselmis suecica, Mantoniella squamata, Ostreococcus tauri, Rubiaceae, such as the genus Coffea, for example the genera and species Cofea spp., Coffea arabica, Coffea canephora or Coffea liberica [coffee], Scrophulariaceae, such as the genus Verbascum, for example the genera and species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [verbascum], Solanaceae, such as the genera Capsicum, Nicotiana, Solanum, Lycopersicon, for example the genera and species Capsicum annuum, Capsicum annuum var. glabriusculum, Capsicum frutescens [pepper], Capsicum annuum [paprika], Nicotiana tabacum, Nicotiana alata, Nicotiana attenuate, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [eggplant], Lycopersicon esculentum, Lycopersicon lycopersicum, Lycopersicon pyriforme, Solanum integrifolium or Solanum lycopersicum [tomato], Sterculiaceae, such as the genus Theobroma, for example the genus and species Theobroma cacao [cacao] or Theaceae, such as the genus Camellia, for example the genus and species Camellia sinensis [tea].

Advantageous microorganisms are, for example, fungi selected from the group of the families Chaetomiaceae, Choanephoraceae, Cryptococcaceae, Cunninghamellaceae, Demetiaceae, Moniliaceae, Mortierellaceae, Mucoraceae, Pythiaceae, Sacharomycetaceae, Saprolegniaceae, Schizosacharomycetaceae, Sodariaceae or Tuberculariaceae.

Examples of microorganisms which may be mentioned are those from the group consisting of: Choanephoraceae, such as the genera Blakeslea, Choanephora, for example the genera and species Blakeslea trispora, Choanephora cucurbitarum, Choanephora infundibulifera var. cucurbitarum, Mortierellaceae, such as the genus Mortierella, for example the genera and species Mortierella isabellina, Mortierella polycephala, Mortierella ramanniana, Mortierella vinacea, Mortierella zonata, Pythiaceae, such as the genera Phytium, Phytophthora, for example the genera and species Pythium debaryanum, Pythium intermedium, Pythium irregulare, Pythium megalacanthum, Pythium paroecandrum, Pythium sylvaticum, Pythium ultimum, Phytophthora cactorum, Phytophthora cinnamomi, Phytophthora citricola, Phytophthora citrophthora, Phytophthora cryptogea, Phytophthora drechsleri, Phytophthora erythroseptica, Phytophthora lateralis, Phytophthora megasperma, Phytophthora nicotianae, Phytophthora nicotianae var. parasitica, Phytophthora palmivora, Phytophthora parasitica, Phytophthora syringae, Saccharomycetaceae, such as the genera Hansenula, Pichia, Saccharomyces, Saccharomycodes, Yarrowia, for example the genera and species Hansenula anomala, Hansenula californica, Hansenula canadensis, Hansenula capsulata, Hansenula ciferrii, Hansenula glucozyma, Hansenula henricii, Hansenula holstii, Hansenula minuta, Hansenula nonfermentans, Hansenula philodendri, Hansenula polymorpha, Hansenula satumus, Hansenula subpelliculosa, Hansenula wickerhamii, Hansenula wingei, Pichia alcoholophila, Pichia angusta, Pichia anomala, Pichia bispora, Pichia burtonii, Pichia canadensis, Pichia capsulata, Pichia carsonii, Pichia cellobiosa, Pichia ciferrii, Pichia farinosa, Pichia fermentans, Pichia finlandica, Pichia glucozyma, Pichia guilliermondii, Pichia haplophila, Pichia henricii, Pichia holstii, Pichia jadinii, Pichia lindnerii, Pichia membranaefaciens, Pichia methanolica, Pichia minuta var. minuta, Pichia minuta var. nonfermentans, Pichia norvegensis, Pichia ohmeri, Pichia pastoris, Pichia philodendri, Pichia pini, Pichia polymorpha, Pichia quercuum, Pichia rhodanensis, Pichia sargentensis, Pichia stipitis, Pichia strasburgensis, Pichia subpelliculosa, Pichia toletana, Pichia trehalophila, Pichia vini, Pichia xylosa, Saccharomyces aceti, Saccharomyces bailii, Saccharomyces bayanus, Saccharomyces bisporus, Saccharomyces capensis, Saccharomyces carlsbergensis, Saccharomyces cerevisiae, Saccharomyces cerevisiae var. ellipsoideus, Saccharomyces chevalieri, Saccharomyces delbrueckii, Saccharomyces diastaticus, Saccharomyces drosophilarum, Saccharomyces elegans, Saccharomyces ellipsoideus, Saccharomyces fermentati, Saccharomyces florentinus, Saccharomyces fragilis, Saccharomyces heterogenicus, Saccharomyces hienipiensis, Saccharomyces inusitatus, Saccharomyces italicus, Saccharomyces kluyveri, Saccharomyces krusei, Saccharomyces lactis, Saccharomyces marxianus, Saccharomyces microellipsoides, Saccharomyces montanus, Saccharomyces norbensis, Saccharomyces oleaceus, Saccharomyces paradoxus, Saccharomyces pastorianus, Saccharomyces pretoriensis, Saccharomyces rosei, Saccharomyces rouxii, Saccharomyces uvarum, Saccharomycodes ludwigii, Yarrowia lipolytica, Schizosaccharomycetaceae such as the genera Schizosaccharomyces e.g. the species Schizosaccharomyces japonicus var. japonicus, Schizosaccharomyces japonicus var. versatilis, Schizosaccharomyces malidevorans, Schizosaccharomyces octosporus, Schizosaccharomyces pombe var. malidevorans, Schizosaccharomyces pombe var. pombe, Thraustochytriaceae such as the genera Althornia, Aplanochytrium, Japonochytrium, Schizochytrium, Thraustochytrium e.g. the species Schizochytrium aggregatum, Schizochytrium limacinum, Schizochytrium mangrovei, Schizochytrium minutum, Schizochytrium octosporum, Thraustochytrium aggregatum, Thraustochytrium amoeboideum, Thraustochytrium antacticum, Thraustochytrium arudimentale, Thraustochytrium aureum, Thraustochytrium benthicola, Thraustochytrium globosum, Thraustochytrium indicum, Thraustochytrium kerguelense, Thraustochytrium kinnei, Thraustochytrium motivum, Thraustochytrium multirudimentale, Thraustochytrium pachydermum, Thraustochytrium proliferum, Thraustochytrium roseum, Thraustochytrium rossii, Thraustochytrium striatum or Thraustochytrium visurgense.

Further advantageous microorganisms are, for example, bacteria selected from the group of the families Bacillaceae, Enterobacteriacae or Rhizobiaceae.

Examples which may be mentioned are the following microorganisms selected from the group consisting of: Bacillaceae, such as the genus Bacillus, for example the genera and species Bacillus acidocaldarius, Bacillus acidoterrestris, Bacillus alcalophilus, Bacillus amyloliquefaciens, Bacillus amylolyticus, Bacillus brevis, Bacillus cereus, Bacillus circulans, Bacillus coagulans, Bacillus sphaericus subsp. fusiformis, Bacillus galactophilus, Bacillus globisporus, Bacillus globisporus subsp. marinus, Bacillus halophilus, Bacillus lentimorbus, Bacillus lentus, Bacillus licheniformis, Bacillus megaterium, Bacillus polymyxa, Bacillus psychrosaccharolyticus, Bacillus pumilus, Bacillus sphaericus, Bacillus subtilis subsp. spizizenii, Bacillus subtilis subsp. subtilis or Bacillus thuringiensis; Enterobacteriacae such as the genera Citrobacter, Edwardsiella, Enterobacter, Erwinia, Escherichia, Klebsiella, Salmonella or Serratia, for example the genera and species Citrobacter amalonaticus, Citrobacter diversus, Citrobacter freundii, Citrobacter genomospecies, Citrobacter gillenii, Citrobacter intermedium, Citrobacter koseri, Citrobacter murliniae, Citrobacter sp., Edwardsiella hoshinae, Edwardsiella ictaluri, Edwardsiella tarda, Erwinia alni, Erwinia amylovora, Erwinia ananatis, Erwinia aphidicola, Erwinia billingiae, Erwinia cacticida, Erwinia cancerogena, Erwinia carnegieana, Erwinia carotovora subsp. atroseptica, Erwinia carotovora subsp. betavasculorum, Erwinia carotovora subsp. odorifera, Erwinia carotovora subsp. wasabiae, Erwinia chrysanthemi, Erwinia cypripedii, Erwinia dissolvens, Erwinia herbicola, Erwinia mallotivora, Erwinia milletiae, Erwinia nigrifluens, Erwinia nimipressuralis, Erwinia persicina, Erwinia psidii, Erwinia pyrifoliae, Erwinia quercina, Erwinia rhapontici, Erwinia rubrifaciens, Erwinia salicis, Erwinia stewartii, Erwinia tracheiphila, Erwinia uredovora, Escherichia adecarboxylata, Escherichia anindolica, Escherichia aurescens, Escherichia blattae, Escherichia coli, Escherichia coli var. communior, Escherichia coli-mutabile, Escherichia fergusonii, Escherichia hermannii, Escherichia sp., Escherichia vulneris, Klebsiella aerogenes, Klebsiella edwardsii subsp. atlantae, Klebsiella omithinolytica, Klebsiella oxytoca, Klebsiella planticola, Klebsiella pneumoniae, Klebsiella pneumoniae subsp. pneumoniae, Klebsiella sp., Klebsiella terrigena, Klebsiella trevisanii, Salmonella abony, Salmonella arizonae, Salmonella bongori, Salmonella choleraesuis subsp. arizonae, Salmonella choleraesuis subsp. bongori, Salmonella choleraesuis subsp. cholereasuis, Salmonella choleraesuis subsp. diarizonae, Salmonella choleraesuis subsp. houtenae, Salmonella choleraesuis subsp. indica, Salmonella choleraesuis subsp. salamae, Salmonella daressalaam, Salmonella enterica subsp. houtenae, Salmonella enterica subsp. salamae, Salmonella enteritidis, Salmonella gallinarum, Salmonella heidelberg, Salmonella panama, Salmonella senftenberg, Salmonella typhimurium, Serratia entomophila, Serratia ficaria, Serratia fonticola, Serratia grimesii, Serratia liquefaciens, Serratia marcescens, Serratia marcescens subsp. marcescens, Serratia marinorubra, Serratia odorifera, Serratia plymouthensis, Serratia plymuthica, Serratia proteamaculans, Serratia proteamaculans subsp. quinovora, Serratia quinivorans or Serratia rubidaea; Rhizobiaceae, such as the genera Agrobacterium, Carbophilus, Chelatobacter, Ensifer, Rhizobium, Sinorhizobium, for example the genera and species Agrobacterium atlanticum, Agrobacterium ferrugineum, Agrobacterium gelatinovorum, Agrobacterium larrymoorei, Agrobacterium meteori, Agrobacterium radiobacter, Agrobacterium rhizogenes, Agrobacterium rubi, Agrobacterium stellulatum, Agrobacterium tumefaciens, Agrobacterium vitis, Carbophilus carboxidus, Chelatobacter heintzii, Ensifer adhaerens, Ensifer arboris, Ensifer fredii, Ensifer kostiensis, Ensifer kummerowiae, Ensifer medicae, Ensifer meliloti, Ensifer saheli, Ensifer terangae, Ensifer xinjiangensis, Rhizobium ciceri, Rhizobium etli, Rhizobium fredii, Rhizobium galegae, Rhizobium gallicum, Rhizobium giardinii, Rhizobium hainanense, Rhizobium huakuii, Rhizobium huautlense, Rhizobium indigoferae, Rhizobium japonicum, Rhizobium leguminosarum, Rhizobium loessense, Rhizobium loti, Rhizobium lupini, Rhizobium mediterraneum, Rhizobium meliloti, Rhizobium mongolense, Rhizobium phaseoli, Rhizobium radiobacter, Rhizobium rhizogenes, Rhizobium rubi, Rhizobium sullae, Rhizobium tianshanense, Rhizobium trifolii, Rhizobium tropici, Rhizobium undicola, Rhizobium vitis, Sinorhizobium adhaerens, Sinorhizobium arboris, Sinorhizobium fredii, Sinorhizobium kostiense, Sinorhizobium kummerowiae, Sinorhizobium medicae, Sinorhizobium meliloti, Sinorhizobium morelense, Sinorhizobium saheli or Sinorhizobium xinjiangense.

Further examples of advantageous microorganisms for the process according to the invention are protists or diatoms selected from the group of the families Dinophyceae, Turaniellidae or Oxytrichidae, such as the genera and species: Crypthecodinium cohnii, Phaeodactylum tricomutum, Stylonychia mytilus, Stylonychia pustulata, Stylonychia putrina, Stylonychia notophora, Stylonychia sp., Colpidium campylum or Colpidium sp.

Those which are advantageously applied in the process according to the invention are transgenic organisms such as fungi, such as Mortierella or Traustrochytrium, yeasts such as Saccharomyces or Schizosaccharomyces, mosses such as Physcomitrella or Ceratodon, nonhuman animals such as Caenorhabditis, algae such as Nephroselmis, Pseudoscourfielda, Prasinococcus, Scherffelia, Tetraselmis, Mantoniella, Ostreococcus, Crypthecodinium or Phaeodactylum or plants such as dicotyledonous or monocotyledonous plants. Organisms which are especially advantageously used in the process according to the invention are organisms which belong to the oil-producing organisms, that is to say which are used for the production of oils, such as fungi, such as Mortierella or Thraustochytrium, algae such as Nephroselmis, Pseudoscourfielda, Prasinococcus, Scherffelia, Tetraselmis, Mantoniella, Ostreococcus, Crypthecodinium, Phaeodactylum, or plants, in particular plants, preferably oil crop plants which comprise large amounts of lipid compounds, such as peanut, oilseed rape, canola, sunflower, safflower (Carthamus tinctoria), poppy, mustard, hemp, castor-oil plant, olive, sesame, Calendula, Punica, evening primrose, verbascum, thistle, wild roses, hazelnut, almond, macadamia, avocado, bay, pumpkin/squash, linseed, soybean, pistachios, borage, trees (oil palm, coconut or walnut) or arable crops such as maize, wheat, rye, oats, triticale, rice, barley, cotton, cassaya, pepper, Tagetes, Solanaceae plants such as potato, tobacco, eggplant and tomato, Vicia species, pea, alfalfa or bushy plants (coffee, cacao, tea), Salix species, and perennial grasses and fodder crops. Preferred plants according to the invention are oil crop plants such as peanut, oilseed rape, canola, sunflower, safflower, poppy, mustard, hemp, castor-oil plant, olive, Calendula, Punica, evening primrose, pumpkin/squash, linseed, soybean, borage, trees (oil palm, coconut). Especially preferred are plants which are high in C18:2- and/or C18:3-fatty acids, such as sunflower, safflower, tobacco, verbascum, sesame, cotton, pumpkin/squash, poppy, evening primrose, walnut, linseed, hemp or thistle. Very especially preferred plants are plants such as safflower, sunflower, poppy, evening primrose, walnut, linseed or hemp.

In principle, all genes of the fatty acid or lipid metabolism can be used in the process for the production of polyunsaturated fatty acids, advantageously in combination with the Δ5-desaturase(s), Δ6-desaturase(s), Δ4-desaturase(s) and/or Δ12-desaturases [for the purposes of the present invention, the plural is understood as encompassing the singular and vice versa]. Genes of the fatty acid or lipid metabolism selected from the group consisting of acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferases, fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene oxide synthases, hydroperoxide lyases or fatty acid elongase(s) are advantageously used in combination with the Δ5-desaturase(s), Δ6-desaturase(s), Δ4-desaturase(s) and/or Δ12-desaturase(s). Genes selected from the group of the Δ4-desaturases, Δ5-desaturases, Δ6-desaturases, Δ9-desaturases, Δ12-desaturases, Δ6-elongases or Δ5-elongases are especially preferably used in combination with the above-mentioned genes for the Δ5-desaturase(s), Δ6-desaturase(s), Δ4-desaturase(s) and/or Δ12-desaturases, it being possible to use individual genes or a plurality of genes in combination.

In comparison with the human elongases, the Δ5-elongases according to the invention have the advantageous property that they do not elongate C₂₂-fatty acids to the corresponding C₂₄-fatty acids. Especially advantageous Δ5-elongases preferentially only convert unsaturated C₂₀-fatty acids. Advantageously, only C₂₀-fatty acids with one double bond in Δ5-position are converted, with ω3-C₂₀-fatty acids being preferred (EPA). In a preferred embodiment of the invention, they furthermore have the property that they have no, or only relatively low, Δ6-elongase activity, in addition to the Δ5-elongase activity. In a yeast feeding test in which EPA had been added to the yeasts to act as substrate, they advantageously convert at least 15% by weight of the added EPA into docosapentaenoic acid (DPA, C22:5^{Δ7,10,13,16,19}), advantageously at least 20% by weight, especially advantageously at least 25% by weight. If γ-linolenic acid (=GLA, C18:3^{Δ6,9,12}) is added as substrate, this substance is advantageously not elongated at all. C18:3^{Δ5,9,12} is likewise not elongated. In another advantageous embodiment, less than 60% by weight, advantageously less than 55% by weight, preferably less than 50% by weight, especially advantageously less than 45% by weight, very especially advantageously less than 40% by weight, of the added GLA are converted into dihomo-γ-linolenic acid (═C20:3^{Δ8,11,14}). In a further, very preferred embodiment of the Δ5-elongase activity according to the invention, GLA is not converted.

In comparison with the known Δ4-desaturases, Δ5-desaturases and Δ6-desaturases, the advantage of the Δ4-desaturases, Δ5-desaturases and Δ6-desaturases according to the invention is that they can convert fatty acids which are bound to phospholipids or CoA-fatty acid esters, advantageously CoA-fatty acid esters.

The Δ12-desaturases used in the process according to the invention advantageously convert oleic acid (C18:1^{Δ9}) into linoleic acid (C18:2^{Δ9,12}) or C18:2^{Δ6,9} into C18:3^{Δ6,9,12} (=GLA). The Δ12-desaturases used advantageously convert fatty acids which are bound to phospholipids or CoA-fatty acid esters, advantageously those which are bound to CoA-fatty acid esters.

Advantageously, the desaturases used in the process according to the invention convert their respective substrates in the form of the CoA-fatty acid esters. If preceded by an elongation step, this advantageously results in an increased product yield. The respective desaturation products are thereby synthesized in greater quantities, since the elongation step is usually carried out with the CoA-fatty acid esters, while the desaturation step is predominantly carried out with the phospholipids or the triglycerides. This fact therefore obviates the need for an exchange reaction between the CoA-fatty acid esters and the phospholipids or triglycerides, which reaction might require a further, potentially limiting, enzymatic reaction.

Owing to the enzymatic activity of the nucleic acids used in the process according to the invention which encode polypeptides with Δ5-desaturase, Δ6-desaturase, Δ4-desaturase, Δ12-desaturase, Δ5-elongase and/or Δ6-elongase activity, advantageously in combination with nucleic acid sequences which encode polypeptides of the fatty acid or lipid metabolism, such as additional polypeptides with Δ4-, Δ5-, Δ6-, Δ12-desaturase or Δ5- or Δ6-elongase activity, a wide range of polyunsaturated fatty acids can be produced in the process according to the invention. Depending on the choice of the organisms, such as the advantageous plants, used for the process according to the invention, mixtures of the various polyunsaturated fatty acids or individual polyunsaturated fatty acids, such as EPA or ARA, can be produced in free or bound form. Depending on the prevailing fatty acid composition in the starting plant (C18:2- or C18:3-fatty acids), fatty acids which are derived from C18:2-fatty acids, such as GLA, DGLA or ARA, or fatty acids which are derived from C18:3-fatty acids, such as SDA, ETA or EPA, are thus obtained. If only linoleic acid (=LA, C18:2^{Δ9,12}) is present as unsaturated fatty acid in the plant used for the process, the process can only afford GLA, DGLA and ARA as products, all of which can be present as free fatty acids or in bound form. If only α-linolenic acid (=ALA, C18:3^{Δ9,12,15}) is present as unsaturated fatty acid in the plant used for the process, as is the case, for example, in linseed, the process can only afford SDA, ETA or EPA and/or DHA as products, all of which can be present as free fatty acids or in bound form, as described above. Owing to the modification of the activity of the enzymes Δ5-desaturase, Δ6-desaturase, Δ4-desaturase, Δ12-desaturase, Δ5-elongase and/or Δ6-elongase which play a role in the synthesis, it is possible to produce, in a targeted fashion, only individual products in the abovementioned organisms, advantageously in the abovementioned plants. Owing to the activity of Δ6-desaturase and Δ6-elongase, for example, GLA and DGLA, or SDA and ETA, are formed, depending on the starting plant and unsaturated fatty acid. DGLA or ETA or mixtures of these are preferably formed. If Δ5-desaturase, Δ5-elongase and Δ4-desaturase are additionally introduced into the organisms, advantageously into the plant, ARA, EPA and/or DHA are additionally formed. Advantageously, only ARA, EPA or DHA or mixtures of these are synthesized, depending on the fatty acid present in the organism, or in the plant, which acts as starting substance for the synthesis. Since biosynthetic cascades are involved, the end products in question are not present in pure form in the organisms. Small amounts of the precursor compounds are always additionally present in the end product. These small amounts amount to less than 20% by weight, advantageously less than 15% by weight, especially advantageously less than 10% by weight, most advantageously less than 5, 4, 3, 2 or 1% by weight, based on the end product DGLA, ETA or their mixtures, or ARA, EPA, DHA or their mixtures, advantageously EPA or DHA or their mixtures.

In addition to the production, directly in the organism, of the starting fatty acids for the Δ5-desaturase, Δ6-desaturase, Δ4-desaturase, Δ12-desaturase, Δ5-elongase and/or Δ6-elongase used in the process of the invention, the fatty acids can also be fed externally. The production in the organism is preferred for reasons of economy. Preferred substrates are linoleic acid (C18:2^{Δ,9,12}), γ-linolenic acid (C18:3^{Δ6,912}), eicosadienoic acid (C20:2^{Δ11,14}), dihomo-γ-linolenic acid (C20:3^{Δ8,11,14}), arachidonic acid (C20:4^{Δ5,8,11,14}), docosatetraenoic acid (C_{22:4}^{Δ7,10,13,16}) and docosapentaenoic acid (C22:5^{Δ4,7,10,13,15}).

To increase the yield in the above-described process for the production of oils and/or triglycerides with an advantageously elevated content of polyunsaturated fatty acids, it is advantageous to increase the amount of starting product for the synthesis of fatty acids; this can be achieved for example by introducing, into the organism, a nucleic acid which encodes a polypeptide with Δ12-desaturase. This is particularly advantageous in oil-producing organisms such as those from the family of the Brassicaceae, such as the genus Brassica, for example oilseed rape; the family of the Elaeagnaceae, such as the genus Elaeagnus, for example the genus and species Olea europaea, or the family Fabaceae, such as the genus Glycine, for example the genus and species Glycine max, which are high in oleic acid. Since these organisms are only low in linoleic acid (Mikoklajczak et al., Journal of the American Oil Chemical Society, 38, 1961, 678-681), the use of the abovementioned Δ12-desaturases for producing the starting product linoleic acid is advantageous.

Nucleic acids used in the process according to the invention are advantageously derived from plants such as algae, for example algae of the family of the Prasinophyceae such as the genera Heteromastix, Mammella, Mantoniella, Micrcmonas, Nephroselmis, Ostreococcus, Prasinocladus, Prasinococcus, Pseudbscourfielda, Pycnococcus, Pyramimonas, Scherffelia or Tetraselmis such as the genera and species Heteromastix longifillis, Mamiella gilva, Mantoniella squamata, Micromonas pusilla, Nephroselmis olivacea, Nephroselmis pyriformis, Nephroselmis rotunda, Ostreococcus tauri, Ostreococcus sp., Prasinocladus ascus, Prasinocladus lubricus, Pycnococcus provasolii, Pyramimonas amylifera, Pyramimonas disomata, Pyramimonas obovata, Pyramimonas orientalis, Pyramimonas parkeae, Pyramimonas spinifera, Pyramimonas sp., Tetraselmis apiculata, Tetraselmis carteriaformis, Tetraselmis chui, Tetraselmis convolutae, Tetraselmis desikacharyl, Tetraselmis gracilis, Tetraselmis hazeni, Tetraselmis impellucida, Tetraselmis inconspicua, Tetraselmis levis, Tetraselmis maculata, Tetraselmis marina, Tetraselmis striata, Tetraselmis subcordiformis, Tetraselmis suecica, Tetraselmis tetrabrachia, Tetraselmis tetrathele, Tetraselmis verrucosa, Tetraselmis verrucosa fo. rubens or Tetraselmis sp. The nucleic acids used are advantageously derived from algae of the genera Mantoniella or Ostreococcus.

Further advantageous plants are algae such as Isochrysis or Crypthecodinium, algae/diatoms such as Thalassiosira, Phaeodactylum or Thraustochytrium, mosses such as Physcomitrella or Ceratodon, or higher plants such as the Primulaceae such as Aleuritia, Calendula stellata, Osteospermum spinescens or Osteospermum hyoseroides, microorganisms such as fungi, such as Aspergillus, Thraustochytrium, Phytophthora, Entomophthora, Mucor or Mortierella, bacteria such as Shewanella, yeasts or animals such as nematodes such as Caenorhabditis, insects or fish. The isolated nucleic acid sequences according to the invention are advantageously derived from an animal of the order of the vertebrates. Preferably, the nucleic acid sequences are derived from the classes of the Vertebrata; Euteleostomi, Actinopterygii; Neopterygii; Teleostei; Euteleostei, Protacanthopterygii, Salmoniformes; Salmonidae or Oncorhynchus. The nucleic acids are especially advantageously derived from fungi, animals, or from plants such as algae or mosses, preferably from the order of the Salmoniformes, such as the family of the Salmonidae, such as the genus Salmo, for example from the genera and species Oncorhynchus mykiss, Trutta trutta or Salmo trutta fario, from algae, such as the genera Mantoniella or Ostreococcus, or from the diatoms such as the genera Thalassiosira or Crypthecodinium.

The process according to the invention advantageously employs the above-mentioned nucleic acid sequences or their derivatives or homologues which encode polypeptides which retain the enzymatic activity of the proteins encoded by nucleic acid sequences. These sequences, individually or in combination with the nucleic acid sequences which encode Δ12-desaturase, Δ4-desaturase, Δ5-desaturase, Δ6-desaturase, Δ5-elongase and/or Δ6-elongase, are cloned into expression constructs and used for the introduction into, and expression in, organisms. Owing to their construction, these expression constructs make possible an advantageous optimal synthesis of the polyunsaturated fatty acids produced in the process according to the invention.

In a preferred embodiment, the process furthermore comprises the step of obtaining a cell or an intact organism which comprises the nucleic acid sequences used in the process, where the cell and/or the organism is transformed with a nucleic acid sequence according to the invention which encodes the Δ12-desaturase, Δ4-desaturase, Δ5-desaturase, Δ6-desaturase, Δ5-elongase and/or Δ6-elongase, a gene construct or a vector as described below, alone or in combination with further nucleic acid sequences which encode proteins of the fatty acid or lipid metabolism. In a further preferred embodiment, this process furthermore comprises the step of obtaining the oils, lipids or free fatty acids from the organism or from the culture. The culture can, for example, take the form of a fermentation culture, for example in the case of the cultivation of microorganisms, such as, for example, Mortierella, Thalassiosira, Mantoniella, Ostreococcus, Saccharomyces or Thraustochytrium, or a greenhouse- or field-grown culture of a plant. The cell or the organism thus produced is advantageously a cell of an oil-producing organism, such as an oil crop, such as, for example, peanut, oilseed rape, canola, linseed, hemp, soybean, safflower, sunflowers or borage.

In the case of plant cells, plant tissue or plant organs, “growing” is understood as meaning, for example, the cultivation on or in a nutrient medium, or of the intact plant on or in a substrate, for example in a hydroponic culture, potting compost or on arable land.

For the purposes of the invention, “transgenic” or “recombinant” means with regard to, for example, a nucleic acid sequence, an expression cassette (=gene construct) or a vector comprising the nucleic acid sequence according to the invention or an organism transformed with the nucleic acid sequences, expression cassettes or vectors according to the invention, all those constructions brought about by recombinant methods in which either
a) the nucleic acid sequence according to the invention, or
b) a genetic control sequence which is operably linked with the nucleic acid sequence according to the invention, for example a promoter, or
c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original organism or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences according to the invention with the corresponding Δ12-desaturase, Δ4-desaturase, Δ5-desaturase, Δ6-desaturase and/or Δ5-elongase genes—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic organism or transgenic plant for the purposes of the invention is therefore understood as meaning, as above, that the nucleic acids used in the process are not at their natural locus in the genome of an organism, it being possible for the nucleic acids to be expressed homologously or heterologously. However, as mentioned, transgenic also means that, while the nucleic acids according to the invention are at their natural position in the genome of an organism, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic organisms are fungi such as Mortierella or Phytophthora, mosses such as Physcomitrella, algae such as Mantoniella or Ostreococcus, diatoms such as Thalassiosira or Crypthecodinium, or plants such as the oil crops.

Organisms or host organisms for the nucleic acids, the expression cassette or the vector used in the process according to the invention are, in principle, advantageously all organisms which are capable of synthesizing fatty acids, specifically unsaturated fatty acids, and/or which are suitable for the expression of recombinant genes. Examples which may be mentioned are plants such as Arabidopsis, Asteraceae such as Calendula or crop plants such as soybean, peanut, castor-oil plant, sunflower, maize, cotton, flax, oilseed rape, coconut, oil palm, safflower (Carthamus tinctorius) or cacao bean, microorganisms, such as fungi, for example the genus Mortierella, Thraustochytrium, Saprolegnia, Phytophthora or Pythium, bacteria, such as the genus Escherichia or Shewanella, yeasts, such as the genus Saccharomyces, cyanobacteria, ciliates, algae such as Mantoniella or Ostreococcus, or protozoans such as dinoflagellates, such as Thalassiosira or Crypthecodinium. Preferred organisms are those which are naturally capable of synthesizing substantial amounts of oil, such as fungi, such as Mortierella alpina, Pythium insidiosum, Phytophthora infestans, or plants such as soybean, oilseed rape, coconut, oil palm, safflower, flax, hemp, castor-oil plant, Calendula, peanut, cacao bean or sunflower, or yeasts such as Saccharomyces cerevisiae, with soybean, flax, oilseed rape, safflower, sunflower, Calendula, Mortierella or Saccharomyces cerevisiae being especially preferred. In principle, host organisms are, in addition to the abovementioned transgenic organisms, also transgenic animals, advantageously nonhuman animals, for example C. elegans.

Further utilizable host cells are detailed in: Goeddel, Gene Expression Technology: Methods in Enzymology 185, Academic Press, San Diego, Calif. (1990).

Expression strains which can be used, for example those with a lower protease activity, are described in: Gottesman, S., Gene Expression Technology: Methods in Enzymology 185, Academic Press, San Diego, Calif. (1990) 119-128.

These include plant cells and certain tissues, organs and parts of plants in all their phenotypic forms such as anthers, fibers, root hairs, stalks, embryos, calli, cotelydons, petioles, harvested material, plant tissue, reproductive tissue and cell cultures which are derived from the actual transgenic plant and/or can be used for bringing about the transgenic plant.

Transgenic plants which comprise the polyunsaturated fatty acids synthesized in the process according to the invention can advantageously be marketed directly without there being any need for the oils, lipids or fatty acids synthesized to be isolated. Plants for the process according to the invention are listed as meaning intact plants and all plant parts, plant organs or plant parts such as leaf, stem, seeds, root, tubers, anthers, fibers, root hairs, stalks, embryos, calli, cotelydons, petioles, harvested material, plant tissue, reproductive tissue and cell cultures which are derived from the transgenic plant and/or can be used for bringing about the transgenic plant. In this context, the seed comprises all parts of the seed such as the seed coats, epidermal cells, seed cells, endosperm or embryonic tissue. However, the compounds produced in the process according to the invention can also be isolated from the organisms, advantageously plants, in the form of their oils, fats, lipids and/or free fatty acids. Polyunsaturated fatty acids produced by this process can be obtained by harvesting the organisms, either from the crop in which they grow, or from the field. This can be done via pressing or extraction of the plant parts, preferably the plant seeds. In this context, the oils, fats, lipids and/or free fatty acids can be obtained by what is known as cold-beating or coldpressing without applying heat. To allow for greater ease of disruption of the plant parts, specifically the seeds, they are previously comminuted, steamed or roasted. The seeds which have been pretreated in this manner can subsequently be pressed or extracted with solvent such as warm hexane. The solvent is subsequently removed. In the case of microorganisms, the latter are, after harvesting, for example extracted directly without further processing steps or else, after disruption, extracted via various methods with which the skilled worker is familiar. In this manner, more than 96% of the compounds produced in the process can be isolated. Thereafter, the resulting products are processed further, i.e. refined. In this process, substances such as the plant mucilages and suspended matter are first removed. What is known as desliming can be effected enzymatically or, for example, chemico-physically by addition of acid such as phosphoric acid. Thereafter, the free fatty acids are removed by treatment with a base, for example sodium hydroxide solution. The resulting product is washed thoroughly with water to remove the alkali remaining in the product and then dried. To remove the pigments remaining in the product, the products are subjected to bleaching, for example using filler's earth or active charcoal. At the end, the product is deodorized, for example using steam.

The PUFAs or LCPUFAs produced by this process are preferably C₁₈-, C₂₀- or C₂₂-fatty acid molecules, advantageously C₂₀- or C₂₂-fatty acid molecules, with at least two double bonds in the fatty acid molecule, preferably three, four, five or six double bonds. These C₁₈-, C₂₀- or C₂₂-fatty acid molecules can be isolated from the organism in the form of an oil, a lipid or a free fatty acid. Suitable organisms are, for example, those mentioned above. Preferred organisms are transgenic plants.

One embodiment of the invention is therefore oils, lipids or fatty acids or fractions thereof which have been produced by the above-described process, especially preferably oil, lipid or a fatty acid composition comprising PUFAs and being derived from transgenic plants.

As described above, these oils, lipids or fatty acids advantageously comprise 6 to 15% of palmitic acid, 1 to 6% of stearic acid, 7-85% of oleic acid, 0.5 to 8% of vaccenic acid, 0.1 to 1% of arachic acid, 7 to 25% of saturated fatty acids, 8 to 85% of monounsaturated fatty acids and 60 to 85% of polyunsaturated fatty acids, in each case based on 100% and on the total fatty acid content of the organisms. Advantageous polyunsaturated fatty acids which are present in the fatty acid esters or fatty acid mixtures are preferably at least 0.1, 0.2, 0.3, 0.4, 0.5, 0.6, 0.7, 0.8, 0.9 or 1% of arachidonic acid, based on the total fatty acid content. Moreover, the fatty acid esters or fatty acid mixtures which have been produced by the process of the invention advantageously comprise fatty acids selected from the group of the fatty acids erucic acid (13-docosaenoic acid), sterculic acid (9,10-methyleneoctadec-9-enoic acid), malvalic acid (8,9-methyleneheptadec-8-enoic acid), chaulmoogric acid (cyclopentenedodecanoic acid), furan fatty acid (9,12-epoxyoctadeca-9,11-dienoic acid), vernolic acid (9,10-epoxyoctadec-12-enoic acid), tariric acid (6-octadecynoic acid), 6-nonadecynoic acid, santalbic acid (t11-octadecen-9-ynoic acid), 6,9-octadecenynoic acid, pyrulic acid (t10-heptadecen-8-ynoic acid), crepenynic acid (9-octadecen-12-ynoic acid), 13,14-dihydrooropheic acid, octadecen-13-ene-9,11-diynoic acid, petroselenic acid (cis-6-octadecenoic acid), 9c,12t-octadecadienoic acid, calendulic acid (8t10t12c-octadecatrienoic acid), catalpic acid (9t11t13c-octadecatrienoic acid), eleostearic acid (9c11t13t-octadecatrienoic acid), jacaric acid (8c10t12c-octadecatrienoic acid), punicic acid (9c11t13c-octadecatrienoic acid), parinaric acid (9c11t13t15c-octadecatetraenoic acid), pinolenic acid (all-cis-5,9,12-octadecatrienoic acid), laballenic acid (5,6-octadecadienallenic acid), ricinoleic acid (12-hydroxyoleic acid) and/or coriolic acid (13-hydroxy-9c,11t-octadecadienoic acid). The abovementioned fatty acids are, as a rule, advantageously only found in traces in the fatty acid esters or fatty acid mixtures produced by the process according to the invention, that is to say that, based on the total fatty acids, they occur to less than 30%, preferably to less than 25%, 24%, 23%, 22% or 21%, especially preferably to less than 20%, 15%, 10%, 9%, 8%, 7%, 6% or 5%, very especially preferably to less than 4%, 3%, 2% or 1%. The fatty acid esters or fatty acid mixtures produced by the process according to the invention advantageously comprise less than 0.1%, based on the total fatty acids, or no butyric acid, no cholesterol, no clupanodonic acid (=docosapentaenoic acid, C22:5^{Δ4,8,12,15,21}) and no nisinic acid (tetracosahexaenoic acid, C23:6^{Δ3,8,12,15,18,21}).

The oils, lipids or fatty acids according to the invention advantageously comprise at least 0.5%, 1%, 2%, 3%, 4% or 5%, advantageously at least 6%, 7%, 8%, 9% or 10%, especially advantageously at least 11%, 12%, 13%, 14% or 15% of ARA or at least 0.5%, 1%, 2%, 3%, 4% or 5%, advantageously at least 6% or 7%, especially advantageously at least 8%, 9% or 10% of EPA and/or DHA, based on the total fatty acid content of the production organism, advantageously of a plant, especially advantageously of an oil crop plant such as soybean, oilseed rape, coconut, oil palm, safflower, flax, hemp, castor-oil plant, Calendula, peanut, cacao bean, sunflower, or the abovementioned further mono- or dicotyledonous oil crop plants.

A further embodiment according to the invention is the use of the oil, lipid, the fatty acids and/or the fatty acid composition in feedstuffs, foodstuffs, cosmetics or pharmaceuticals. The oils, lipids, fatty acids or fatty acid mixtures according to the invention can be used in the manner with which the skilled worker is familiar for mixing with other oils, lipids, fatty acids or fatty acid mixtures of animal origin, such as, for example, fish oils. These oils, lipids, fatty acids or fatty acid mixtures, which are composed of vegetable and animal constituents, may also be used for the preparation of feedstuffs, foodstuffs, cosmetics or pharmaceuticals.

The term “oil”, “lipid” or “fat” is understood as meaning a fatty acid mixture comprising unsaturated, saturated, preferably esterified, fatty acid(s). The oil, lipid or fat is preferably high in polyunsaturated free or, advantageously, esterified fatty acid(s), in particular linoleic acid, γ-linolenic acid, dihomo-γ-linolenic acid, arachidonic acid, α-linolenic acid, stearidonic acid, eicosatetraenoic acid, eicosapentaenoic acid, docosapentaenoic acid or docosahexaenoic acid. The amount of unsaturated esterified fatty acids preferably amounts to approximately 30%, a content of 50% is more preferred, a content of 60%, 70%, 80% or more is even more preferred. For the analysis, the fatty acid content can, for example, be determined by gas chromatography after converting the fatty acids into the methyl esters by transesterification. The oil, lipid or fat can comprise various other saturated or unsaturated fatty acids, for example calendulic acid, palmitic acid, palmitoleic acid, stearic acid, oleic acid and the like. The content of the various fatty acids in the oil or fat can vary, in particular depending on the starting organism.

The polyunsaturated fatty acids with advantageously at least two double bonds which are produced in the process are, as described above, for example sphingolipids, phosphoglycerides, lipids, glycolipids, phospholipids, monoacylglycerol, diacylglycerol, triacylglycerol or other fatty acid esters.

Starting from the polyunsaturated fatty acids with advantageously at least five or six double bonds, which acids have been prepared in the process according to the invention, the polyunsaturated fatty acids which are present can be liberated for example via treatment with alkali, for example aqueous KOH or NaOH, or acid hydrolysis, advantageously in the presence of an alcohol such as methanol or ethanol, or via enzymatic cleavage, and isolated via, for example, phase separation and subsequent acidification via, for example, H₂SO₄. The fatty acids can also be liberated directly without the above-described processing step.

After their introduction into an organism, advantageously a plant cell or plant, the nucleic acids used in the process can either be present on a separate plasmid or, advantageously, integrated into the genome of the host cell. In the case of integration into the genome, integration can be random or else be effected by recombination such that the native gene is replaced by the copy introduced, whereby the production of the desired compound by the cell is modulated, or by the use of a gene in trans, so that the gene is linked operably with a functional expression unit which comprises at least one sequence which ensures the expression of a gene and at least one sequence which ensures the polyadenylation of a functionally transcribed gene. The nucleic acids are advantageously introduced into the organisms via multiexpression cassettes or constructs for multiparallel expression, advantageously into the plants for the multiparallel seed-specific expression of genes.

Mosses and algae are the only known plant systems which produce substantial amounts of polyunsaturated fatty acids such as arachidonic acid (ARA) and/or eicosapentaenoic acid (EPA) and/or docosahexaenoic acid (DHA). Mosses comprise PUFAs in membrane lipids, while algae, organisms which are related to algae and a few fungi also accumulate substantial amounts of PUFAs in the triacylglycerol fraction. This is why nucleic acid molecules which are isolated from such strains that also accumulate PUFAs in the triacylglycerol fraction are particularly advantageous for the process according to the invention and thus for the modification of the lipid and PUFA production system in a host, in particular plants such as oil crops, for example oilseed rape, canola, linseed, hemp, soybeans, sunflowers and borage. They can therefore be used advantageously in the process according to the invention.

Substrates which are advantageously suitable for the nucleic acids which are used in the process according to the invention and which encode polypeptides with Δ12-desaturase, Δ5-desaturase, Δ4-desaturase, Δ6-desaturase, Δ5-elongase and/or Δ6-elongase activity and/or the further nucleic acids used, such as the nucleic acids which encode polypeptides of the fatty acid or lipid metabolism selected from the group acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene oxide synthases, hydroperoxide lyases or fatty acid elongase(s) are advantageously C₁₆, C₁₈- or C₂₀-fatty acids. The fatty acids converted as substrates in the process are preferably converted in the form of their acyl-CoA esters and/or their phospholipid esters.

To produce the long-chain PUFAs according to the invention, the polyunsaturated C₁₈-fatty acids must first be desaturated by the enzymatic activity of a desaturase and subsequently be elongated by at least two carbon atoms via an elongase. After one elongation cycle, this enzyme activity gives C₂₀-fatty acids and after two-elongation cycles C₂₂-fatty acids. The activity of the desaturases and elongases used in the process according to the invention preferably leads to C₁₈-, C₂₀- and/or C₂₂-fatty acids, advantageously with at least two double bonds in the fatty acid molecule, preferably with three, four, five or six double bonds, especially preferably to give C₂₀- and/or C₂₂-fatty acids with at least two double bonds in the fatty acid molecule, preferably with three, four, five or six double bonds, very especially preferably with five or six double bonds in the molecule. After a first desaturation and the elongation have taken place, further desaturation and elongation steps such as, for example, such a desaturation in the Δ5 and Δ4 position may take place. Products of the process according to the invention which are especially preferred are dihomo-γ-linolenic acid, arachidonic acid, eicosapentaenoic acid, docosapentaenoic acid and/or docosahexaenoic acid. The C₂₀-fatty acids with at least two double bonds in the fatty acid can be elongated by the enzymatic activity according to the invention in the form of the free fatty acid or in the form of the esters, such as phospholipids, glycolipids, sphingolipids, phosphoglycerides, monoacylglycerol, diacylglycerol or triacylglycerol.

The preferred biosynthesis site of the fatty acids, oils, lipids or fats in the plants which are advantageously used is, for example, in general the seed or cell strata of the seed, so that seed-specific expression of the nucleic acids used in the process makes sense. However, it is obvious that the biosynthesis of fatty acids, oils or lipids need not be limited to the seed tissue, but can also take place in a tissue-specific manner in all the other parts of the plant, for example in epidermal cells or in the tubers.

If microorganism such as yeasts, such as Saccharomyces or Schizosaccharomyces, fungi such as Mortierella, Aspergillus, Phytophthora, Entomophthora, Mucor or Thraustochytrium, algae such as Isochrysis, Mantoniella, Ostreococcus, Phaeodactylum or Crypthecodinium are used as organisms in the process according to the invention, these organisms are advantageously grown in fermentation cultures.

Owing to the use of the nucleic acids according to the invention which encode a Δ5-elongase, the polyunsaturated fatty acids produced in the process can be increased by at least 5%, preferably by at least 10%, especially preferably by at least 20%, very especially preferably by at least 50% in comparison with the wild types of the organisms which do not comprise the nucleic acids recombinantly.

In principle, the polyunsaturated fatty acids produced by the process according to the invention in the organisms used in the process can be increased in two different ways. Advantageously, the pool of free polyunsaturated fatty acids and/or the content of the esterified polyunsaturated fatty acids produced via the process can be enlarged. Advantageously, the pool of esterified polyunsaturated fatty acids in the transgenic organisms is enlarged by the process according to the invention.

If microorganisms are used as organisms in the process according to the invention, they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while passing in oxygen. The pH of the nutrient liquid can either be kept constant, that is to say regulated during the culturing period, or not. The cultures can be grown batchwise, semi-batchwise or continuously. Nutrients can be provided at the beginning of the fermentation or fed in semicontinuously or continuously. The polyunsaturated fatty acids produced can be isolated from the organisms as described above by processes known to the skilled worker, for example by extraction, distillation, crystallization, if appropriate precipitation with salt, and/or chromatography. To this end, the organisms can advantageously be disrupted beforehand.

If the host organisms are microorganisms, the process according to the invention is advantageously carried out at a temperature of between 0° C. and 95° C., preferably between 10° C. and 85° C., especially preferably between 15° C. and 75° C., very especially preferably between 15° C. and 45° C.

In this process, the pH value is advantageously kept between pH 4 and 12, preferably between pH 6 and 9, especially preferably between pH 7 and 8.

The process according to the invention can be operated batchwise, semibatchwise or continuously. An overview over known cultivation methods can be found in the textbook by Chmiel (Bioprozeβtechnik 1. Einführung in die Bioverfahrenstechnik [Bioprocess technology 1. Introduction to bioprocess technology] (Gustav Fischer Verlag, Stuttgart, 1991)) or in the textbook by Storhas (Bioreaktoren und periphere Einrichtungen [Bioreactors and peripheral equipment] (Vieweg Verlag, Braunschweig/Wiesbaden, 1994)).

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar raffination. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogen phosphate or dipotassium hydrogen phosphate or the corresponding sodium-containing salts may be used as sources of phosphorus.

Chelating agents may be added to the medium in order to keep the metal ions in solution. Particularly suitable chelating agents include dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air, into the culture. The temperature of the culture is normally 20° to 45° C. and preferably 25° C. to 40° C. The culture is continued until formation of the desired product is at a maximum. This aim is normally achieved within 10 to 160 hours.

The fermentation broths obtained in this way, in particular those containing polyunsaturated fatty acids, usually contain a dry mass of from 7.5 to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the fatty acids present therein.

The fatty acids obtained in the process are also suitable as starting material for the chemical synthesis of further products of interest. For example, they can be used in combination with one another or alone for the preparation of pharmaceuticals, foodstuffs, animal feeds or cosmetics.

The invention furthermore relates to isolated nucleic acid sequences encoding a polypeptide with Δ6-desaturase activity, selected from the group consisting of:
a) a nucleic acid sequence with the sequence shown in SEQ ID NO:13, or
b) nucleic acid sequences which, as the result of the degeneracy of the genetic code, can be derived from the amino acid sequence shown in SEQ ID NO:14, or
c) derivatives of the nucleic acid sequence shown in SEQ ID NO:13 which encode polypeptides with at least 40% homology at the amino acid level with SEQ ID NO:14 and which have Δ6-desaturase activity.

The invention furthermore relates to isolated nucleic acid sequences encoding a polypeptide with Δ5-desaturase activity, selected from the group consisting of:
a) a nucleic acid sequence with the sequence shown in SEQ ID NO:9 or in SEQ ID NO:11,
b) nucleic acid sequences which, as the result of the degeneracy of the genetic code, can be derived from the amino acid sequence shown in SEQ ID NO:10 or in SEQ ID NO:12, or
c) derivatives of the nucleic acid sequence shown in SEQ ID NO:9 or in SEQ ID NO:11 which encode polypeptides with at least 40% homology at the amino acid level with SEQ ID NO:10 or in SEQ ID NO:12 and which have Δ5-desaturase activity.

The invention furthermore relates to isolated nucleic acid sequences encoding a polypeptide with Δ4-desaturase activity, selected from the group consisting of:
a) a nucleic acid sequence with the sequence shown in SEQ ID NO:7,
b) nucleic acid sequences which, as the result of the degeneracy of the genetic code, can be derived from the amino acid sequence shown in SEQ ID NO:8, or
c) derivatives of the nucleic acid sequence shown in SEQ ID NO:7 which encode polypeptides with at least 40% homology at the amino acid level with SEQ ID NO:8 and which have Δ4-desaturase activity.

The invention furthermore relates to isolated nucleic acid sequences encoding a polypeptide with Δ12-desaturase activity, selected from the group consisting of:
a) a nucleic acid sequence with the sequence shown in SEQ ID NO:15,
b) nucleic acid sequences which, as the result of the degeneracy of the genetic code, can be derived from the amino acid sequence shown in SEQ ID NO:16, or
c) derivatives of the nucleic acid sequence shown in SEQ ID NO:15 which encode polypeptides with at least 50% homology at the amino acid level with SEQ ID NO:16 and which have Δ12-desaturase activity.

The invention furthermore relates to gene constructs which comprise the nucleic acid sequences SEQ ID NO:7, SEQ ID NO:9, SEQ ID NO:11, SEQ ID NO:13 or SEQ ID NO:15 according to the invention, wherein the nucleic acid is linked operably with one or more regulatory signals. In addition, additional biosynthesis genes of the fatty acid or lipid metabolism selected from the group acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene oxide synthases, hydroperoxide lyases or fatty acid elongase(s) may be present in the gene construct. Advantageously, biosynthesis genes of the fatty acid or lipid metabolism selected from the group Δ4-desaturase, Δ5-desaturase, Δ6-desaturase, Δ9-desaturase, Δ12-desaturase or Δ6-elongase are additionally present.

All of the nucleic acid sequences used in the process according to the invention are advantageously derived from a eukaryotic organism such as a plant, a microorganism or an animal. The nucleic acid sequences are preferably derived from the order Salmoniformes, algae such as Mantoniella or Ostreococcus, fungi such as the genus Phytophthora or from diatoms such as the genera Thalassiosira or Crypthecodinium.

The nucleic acid sequences used in the process which encode proteins with Δ4-desaturase, Δ5-desaturase, Δ6-desaturase, Δ9-desaturase, Δ112-desaturase, Δ5-elongase or Δ6-elongase activity are advantageously introduced alone or, preferably, in combination in an expression cassette (=nucleic acid construct) which makes possible the expression of the nucleic acids in an organism, advantageously a plant or a microorganism. The nucleic acid construct can comprise more than one nucleic acid sequence with an enzymatic activity, such as, for example, of a Δ12-desaturase, Δ4-desaturase, Δ5-desaturase, Δ6-desaturase, Δ5-elongase and/or Δ6-elongase.

To introduce the nucleic acids used in the process, the latter are advantageously amplified and ligated in the known manner. Preferably, a procedure following the protocol for Pfu DNA polymerase or a Pfu/Taq DNA polymerase mixture is followed. The primers are selected taking into consideration the sequence to be amplified. The primers should advantageously be chosen in such a way that the amplificate comprises the entire codogenic sequence from the start codon to the stop codon. After the amplification, the amplificate is expediently analyzed. For example, a gelelectrophoretic separation can be carried out, which is followed by a quantitative and a qualitative analysis. Thereafter, the amplificate can be purified following a standard protocol (for example Qiagen). An aliquot of the purified amplificate is then available for the subsequent cloning step. Suitable cloning vectors are generally known to the skilled worker. These include, in particular, vectors which are capable of replication in microbial systems, that is to say mainly vectors which ensure efficient cloning in yeasts or fungi and which make possible the stable transformation of plants. Those which must be mentioned in particular are various binary and cointegrated vector systems which are suitable for the T-DNA-mediated transformation. Such vector systems are, as a rule, characterized in that they comprise at least the vir genes required for the Agrobacterium-mediated transformation and the T-DNA-delimiting sequences (T-DNA border). These vector systems preferably also comprise further cis-regulatory regions such as promoters and terminator sequences and/or selection markers, by means of which suitably transformed organisms can be identified. While in the case of cointegrated vector systems vir genes and T-DNA sequences are arranged on the same vector, binary systems are based on at least two vectors, one of which bears vir genes, but no T-DNA, while a second one bears T-DNA, but no vir gene. Owing to this fact, the last-mentioned vectors are relatively small and easy to manipulate and to replicate both in E. coli and in Agrobacterium. These binary vectors include vectors from the series pBIB-HYG, pPZP, pBecks, pGreen. In accordance with the invention, Bin19, pBI101, pBinAR, pGPTV and pCAMBIA are used by preference. An overview of the binary vectors and their use is found in Hellens et al, Trends in Plant Science (2000) 5, 446-451. In order to prepare the vectors, the vectors can first be linearized, with restriction endonuclease(s) and then modified enzymatically in a suitable manner. Thereafter, the vector is purified, and an aliquot is employed for the cloning step. In the cloning step, the enzymatically cleaved and, if appropriate, purified amplificate is cloned with vector fragments which have been prepared in a similar manner, using ligase. In this context, a particular nucleic acid construct, or vector or plasmid construct, can have one or else more than one codogenic gene segment. The codogenic gene segments in these constructs are preferably linked operably with regulatory sequences. The regulatory sequences include, in particular, plant sequences such as the above-described promoters and terminator sequences. The constructs can advantageously be stably propagated in microorganisms, in particular in Escherichia coli and Agrobacterium tumefaciens, under selective conditions and make possible the transfer of heterologous DNA into plants or microorganisms.

The nucleic acids used in the process, the inventive nucleic acids and nucleic acid constructs, can be introduced into organisms such as microorganisms or advantageously plants, advantageously using cloning vectors, and thus be used in the transformation of plants such as those which are published and cited in: Plant Molecular Biology and Biotechnology (CRC Press, Boca Raton, Fla.), Chapter 6/7, p. 71-119 (1993); F. F. White, Vectors for Gene Transfer in Higher Plants; in: Transgenic Plants, Vol. 1, Engineering and Utilization, Ed.: Kung and R. Wu, Academic Press, 1993, 15-38; B. Jenes et al., Techniques for Gene Transfer, in: Transgenic Plants, Vol. 1, Engineering and Utilization, Ed.: Kung and R. Wu, Academic Press (1993), 128-143; Potrykus, Annu. Rev. Plant Physiol. Plant Molec. Biol. 42 (1991.), 205-225. Thus, the nucleic acids, the inventive nucleic acids and nucleic acid constructs, and/or vectors used in the process can be used for the recombinant modification of a broad spectrum of organisms, advantageously plants, so that the latter become better and/or more efficient PUFA producers.

A series of mechanisms by which a modification of the Δ12-desaturase, Δ5-elongase, Δ6-elongase, Δ5-desaturase, Δ4-desaturase and/or Δ6-desaturase protein and of the further proteins used in the process, such as Δ12-desaturase, Δ6-desaturase, Δ6-elongase, Δ5-desaturase or Δ4-desaturase proteins, is possible exist, so that the yield, production and/or production efficiency of the advantageous polyunsaturated fatty acids in a plant, preferably in an oil crop plant or a microorganism, can be influenced directly owing to this modified protein. The number or activity of the Δ12-desaturase, Δ6-desaturase, Δ6-elongase, Δ5-desaturase, Δ5-elongase or Δ4-desaturase proteins or genes can be increased, so that greater amounts of the gene products and, ultimately, greater amounts of the compounds of the general formula I are produced. A de novo synthesis in an organism which has lacked the activity and ability to biosynthesize the compounds prior to introduction of the corresponding gene(s) is also possible. This applies analogously to the combination with further desaturases or elongases or further enzymes of the fatty acid and lipid metabolism. The use of various divergent sequences, i.e. sequences which differ at the DNA sequence level, may also be advantageous in this context, or else the use of promoters for gene expression which make possible a different gene expression in the course of time, for example as a function of the degree of maturity of a seed or an oilstoring tissue.

Owing to the introduction of a Δ112-desaturase, Δ6-desaturase, Δ6-elongase, Δ5-desaturase, Δ5-elongase and/or Δ4-desaturase gene into an organism, alone or in combination with other genes in a cell, it is not only possible to increase biosynthesis flux towards the end product, but also to increase, or to create de novo the corresponding triacylglycerol composition. Likewise, the number or activity of other genes which are involved in the import of nutrients which are required for the biosynthesis of one or more fatty acids, oils, polar and/or neutral lipids, can be increased, so that the concentration of these precursors, cofactors or intermediates within the cells or within the storage compartment is increased, whereby the ability of the cells to produce PUFAs as described below is enhanced further. By optimizing the activity or increasing the number of one or more Δ12-desaturase, Δ6-desaturase, Δ6-elongase, Δ5-desaturase, Δ5-elongase or Δ4-desaturase genes which are involved in the biosynthesis of these compounds, or by destroying the activity of one or more genes which are involved in the degradation of these compounds, an enhanced yield, production and/or efficiency of production of fatty acid and lipid molecules in organisms, advantageously in plants, is made possible.

The isolated nucleic acid molecules used in the process according to the invention encode proteins or parts of these, where the proteins or the individual protein or parts thereof comprise(s) an amino acid sequence with sufficient homology to an amino acid sequence which is shown in the sequences SEQ ID NO:2, SEQ ID NO:4, SEQ ID NO:6, SEQ ID NO:8, SEQ ID NO:10, SEQ ID NO:12, SEQ ID NO:14 or SEQ ID NO:16, so that the proteins or parts thereof retain a Δ12-desaturase, Δ6-desaturase, Δ6-elongase, Δ5-desaturase, Δ5-elongase or Δ4-desaturase activity. The proteins or parts thereof which is/are encoded by the nucleic acid molecule(s) preferably retains their essential enzymatic activity and the ability of participating in the metabolism of compounds required for the synthesis of cell membranes or lipid bodies in organisms, advantageously in plants, or in the transport of molecules across these membranes. Advantageously, the proteins encoded by the nucleic acid molecules have at least approximately 40%, preferably at least approximately 50% or 60% and more preferably at least approximately 70%, 80% or 90% and most preferably at least approximately 85%, 86%, 87%, 88%, 89%, 90%, 91%, 92%, 93%, 94%, 95%, 96%, 97%, 98%, 99% or more identity with the amino acid sequences shown in SEQ ID NO:2, SEQ ID NO:4, SEQ ID NO:6, SEQ ID NO:8, SEQ ID NO:10, SEQ ID NO:12, SEQ ID NO:14 or SEQ ID NO:16. For the purposes of the invention, homology or homologous is understood as meaning identity or identical, respectively.

The homology was calculated over the entire amino acid or nucleic acid sequence region. The skilled worker has available a series of programs which are based on various algorithms for the comparison of various sequences. Here, the algorithms of Needleman and Wunsch or Smith and Waterman give particularly reliable results. The program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970) and Smith and Waterman (Adv. Appl. Math. 2; 482-489 (1981)], which are part of the GCG software packet [Genetics Computer Group, 575 Science Drive, Madison, Wis., USA 53711 (1991)], were used for the sequence alignment. The sequence homology values which are indicated above as a percentage were determined over the entire sequence region using the program GAP and the following settings: Gap Weight: 50, Length Weight: 3, Average Match: 10.000 and Average Mismatch: 0.000. Unless otherwise specified, these settings were always used as standard settings for the sequence alignments.

Essential enzymatic activity of the Δ12-desaturase, Δ6-desaturase, Δ6-elongase, Δ5-desaturase, Δ5-elongase or Δ4-desaturase used in the process according to the invention is understood as meaning that they retain at least an enzymatic activity of at least 10%, preferably 20%, especially preferably 30% and very especially 40% in comparison with the proteins/enzymes encoded by the sequence SEQ ID NO:1, SEQ ID NO:3, SEQ ID NO:5, SEQ ID NO:7, SEQ ID NO:9, SEQ ID NO:11, SEQ ID NO:13 or SEQ ID NO:15 and their derivatives and can thus participate in the metabolism of compounds required for the synthesis of fatty acids, fatty acid esters such as diacylglycerides and/or triacylglycerides in an organism, advantageously a plant or a plant cell, or in the transport of molecules across membranes, meaning C₁₈-, C₂₀- or C₂₂-carbon chains in the fatty acid molecule with double bonds at least two, advantageously three, four, five or six positions.

Nucleic acids which can advantageously be used in the process are derived from bacteria, fungi, diatoms, animals such as Caenorhabditis or Oncorhynchus or plants such as algae or mosses, such as the genera Shewanella, Physcomitrella, Thraustochytrium, Fusarium, Phytophthora, Ceratodon, Mantoniella, Ostreococcus, Isochrysis, Aleurita, Muscarioides, Mortierella, Borago, Phaeodactylum, Crypthecodinium, specifically from the genera and species Oncorhynchus mykiss, Thalassiosira pseudonona, Mantoniella squamata, Ostreococcus sp., Ostreococcus tauri, Euglena gracilis, Physcomitrella patens, Phytophthora infestans, Fusarium graminaeum, Cryptocodinium cohnii, Ceratodon purpureus, Isochrysis galbana, Aleurita farinosa, Thraustochytrium sp., Muscarioides viallii, Mortierella alpina, Borago officinalis, Phaeodactylum tricornutum, Caenorhabditis elegans or especially advantageously from Oncorhynchus mykiss, Thalassiosira pseudonona or Crypthecodinium cohnii.

Alternatively, nucleic acid sequences which encode a Δ12-desaturase, Δ6-desaturase, Δ6-elongase, Δ5-desaturase, Δ5-elongase or Δ4-desaturase and which advantageously hybridize under stringent conditions with a nucleic acid sequence as shown in SEQ ID NO:1, SEQ ID NO:3, SEQ ID NO:5, SEQ ID NO:7, SEQ ID NO:9, SEQ ID NO:11, SEQ ID NO:13 or SEQ ID NO:15 can be used in the process according to the invention.

The nucleic acid sequences used in the process are advantageously introduced into an expression cassette which makes possible the expression of the nucleic acids in organisms such as microorganisms or plants.

In doing so, the nucleic acid sequences which encode Δ12-desaturase, Δ6-desaturase, Δ6-elongase, Δ5-desaturase, Δ5-elongase or Δ4-desaturase are linked operably with one or more regulatory signals, advantageously for enhancing gene expression. These regulatory sequences are intended to make possible the specific expression of the genes and proteins. Depending on the host organism, this may mean, for example, that the gene is expressed and/or overexpressed only after induction has taken place, or else that it is expressed and/or overexpressed immediately. For example, these regulatory sequences take the form of sequences to which inductors or repressors bind, thus controlling the expression of the nucleic acid. In addition to these novel regulatory sequences, or instead of these sequences, the natural regulation of these sequences may still be present before the actual structural genes and, if appropriate, may have been genetically modified in such a way that their natural regulation is eliminated and the expression of the genes is enhanced. However, the expression cassette (=expression construct=gene construct) can also be simpler in construction, that is to say no additional regulatory signals have been inserted before the nucleic acid sequence or its derivatives, and the natural promoter together with its regulation was not removed. Instead, the natural regulatory sequence has been mutated in such a way that regulation no longer takes place and/or gene expression is enhanced. These modified promoters can also be positioned on their own before the natural gene in the form of part-sequences (=promotor with parts of the nucleic acid sequences in accordance with the invention) in order to enhance the activity. Moreover, the gene construct may advantageously also comprise one or more what are known as enhancer sequences in operable linkage with the promoter, which make possible an enhanced expression of the nucleic acid sequence. Additional advantageous sequences, such as further regulatory elements or terminator sequences, may also be inserted at the 3′ end of the DNA sequences. The Δ112-desaturase, Δ4-desaturase, Δ5-desaturase, Δ6-desaturase, Δ5-elongase and/or Δ6-elongase genes may be present in one or more copies of the expression cassette (=gene construct). Preferably, only one copy of the genes is present in each expression cassette. This gene construct or the gene constructs can be expressed together in the host organism. In this context, the gene construct(s) can be inserted in one or more vectors and be present in the cell in free form, or else be inserted in the genome. It is advantageous for the insertion of further genes in the host genome when the genes to be expressed are present together in one gene construct.

In this context, the regulatory sequences or factors can, as described above, preferably have a positive effect on the gene expression of the genes introduced, thus enhancing it. Thus, an enhancement of the regulatory elements, advantageously at the transcriptional level, may take place by using strong transcription signals such as promoters and/or enhancers. In addition, however, enhanced translation is also possible, for example by improving the stability of the mRNA.

A further embodiment of the invention is one or more gene constructs which comprise one or more sequences which are defined by SEQ ID NO:1, SEQ ID NO:3, SEQ ID NO:5, SEQ ID NO:7, SEQ ID NO:9, SEQ ID NO:11, SEQ ID NO:13 or SEQ ID NO:15 or its derivatives and which encode polypeptides as shown in SEQ ID NO:2, SEQ ID NO:4, SEQ ID NO:6, SEQ ID NO:8, SEQ ID NO:10, SEQ ID NO:12, SEQ ID NO:14 or SEQ ID NO:16. The abovementioned Δ12-desaturase, Δ6-desaturase, Δ6-elongase, Δ5-desaturase, Δ5-elongase or Δ4-desaturase proteins lead advantageously to a desaturation or elongation of fatty acids, the substrate advantageously having one, two, three, four, five or six double bonds and advantageously 18, 20 or 22 carbon atoms in the fatty acid molecule. The same applies to their homologs, derivatives or analogs, which are linked operably with one or more regulatory signals, advantageously for enhancing gene expression.

Advantageous regulatory sequences for the novel process are present for example in promoters such as the cos, tac, trp, tet, trp-tet, lpp, lac, lpp-lac, lacIq, T7, T5, T3, gal, trc, ara, SP6, λ-PR or λ-PL promoter and are advantageously employed in Gramnegative bacteria. Further advantageous regulatory sequences are, for example, present in the Gram-positive promoters amy and SPO2, in the yeast or fungal promoters ADC1, MFα, AC, P-60, CYC1, GAPDH, TEF, rp28, ADH or in the plant promoters CaMV/35S [Franck et al., Cell 21 (1980) 285-294], PRP1 [Ward et al., Plant. Mol. Biol. 22 (1993)], SSU, OCS, lib4, usp, STLS1, B33, nos or in the ubiquitin or phaseolin promoter. Advantageous in this context are also inducible promoters, such as the promoters described in EP-A-0 388 186 (benzenesulfonamide-inducible), Plant J. 2, 1992:397-404 (Gatz et al., tetracycline-inducible), EP-A-0 335 528 (abscissic acid-inducible) or WO 93/21334 (ethanol- or cyclohexenol-inducible) promoters. Further suitable plant promoters are the cytosolic FBPase promoter or the ST-LSI promoter of potato (Stockhaus et al., EMBO J. 8,1989, 2445), the glycine max phosphoribosylpyrophosphate amidotransferase promoter (Genbank Accession No. U87999) or the node-specific promoter described in EP-Δ0 249 676. Especially advantageous promoters are promoters which make possible the expression in tissues which are involved in the biosynthesis of fatty acids. Very especially advantageous are seed-specific promoters, such as the USP promoter as described, but also other promoters such as the LeB4, DC3, phaseolin or napin promoter. Further especially advantageous promoters are seed-specific promoters which can be used for monocotyledonous or dicotyledonous plants and which are described in U.S. Pat. No. 5,608,152 (oilseed rape napin promoter), WO 98/45461 (Arabidopsis oleosin promoter), U.S. Pat. No. 5,504,200 (Phaseolus vulgaris phaseolin promoter), WO 91/13980 (Brassica Bce4 promoter), by Baeumlein et al., Plant J., 2, 2, 1992:233-239 (LeB4 promoter from a legume), these promoters being suitable for dicots. Examples of promoters which are suitable for monocots are the barley lpt-2 or lpt-1 promoter (WO 95/15389 and WO 95/23230), the barley hordein promoter and other suitable promoters described in WO 99/16890.

In principle, it is possible to use all natural promoters together with their regulatory sequences, such as those mentioned above, for the novel process. It is also possible and advantageous to use synthetic promoters, either in addition or alone, in particular when they mediate seed-specific expression, such as those described in WO 99/16890.

In order to achieve a particularly high PUFA content, especially in transgenic plants, the PUFA biosynthesis genes should advantageously be expressed in oil crops in a seed-specific manner. To this end, seed-specific promoters can be used, or those promoters which are active in the embryo and/or in the endosperm. In principle, seed-specific promoters can be isolated both from dicotyledonous and from monocotyledonous plants. Advantageous preferred promoters are listed hereinbelow: USP (=unknown seed protein) and vicilin (Vicia faba) [Baumlein et al., Mol. Gen Genet., 1991, 225(3)], napin (oilseed rape) [U.S. Pat. No. 5,608,152], acyl carrier protein (oilseed rape) [U.S. Pat. No. 5,315,001 and WO 92/18634], oleosin (Arabidopsis thaliana) [WO 98/45461 and WO 93/20216], phaseolin (Phaseolus vulgaris) [U.S. Pat. No. 5,504,200], Bce4 [WO 91/13980], legumines B4 (LegB4 promoter) [Bäumlein et al., Plant J., 2, 2, 1992], Lpt2 and lpt1 (barley) [WO 95/15389 and WO95/23230], seed-specific promoters from rice, maize and wheat [WO 99/16890], Amy32b, Amy 6-6 and aleurain [U.S. Pat. No. 5,677,474], Bce4 (oilseed rape) [U.S. Pat. No. 5,530,149], glycinin (soybean). [EP 571 741], phosphoenol pyruvate carboxylase (soybean) [JP 06/62870], ADR12-2 (soybean) [WO 98/08962], isocitrate lyase (oilseed rape) [U.S. Pat. No. 5,689,040] or α-amylase (barley) [EP 781 849].

Plant gene expression can also be facilitated via a chemically inducible promoter (see review in Gatz 1997, Annu. Rev. Plant Physiol. Plant Mol. Biol., 48:89-108). Chemically inducible promoters are particularly suitable when it is desired that gene expression should take place in a time-specific manner. Examples of such promoters are a salicylic acid-inducible promoter (WO 95/19443), a tetracycline-inducible promoter (Gatz et al. (1992) Plant J. 2, 397-404) and an ethanol-inducible promoter.

To ensure the stable integration of the biosynthesis genes into the transgenic plant over a plurality of generation, each of the nucleic acids which encode Δ12-desaturase, Δ6-desaturase, Δ6-elongase, Δ5-desaturase, Δ5-elongase and/or Δ4-desaturase and which are used in the process should be expressed under the control of a separate promoter, preferably a promoter which differs from the other promoters, since repeating sequence motifs can lead to instability of the T-DNA, or to recombination events. In this context, the expression cassette is advantageously constructed in such a way that a promoter is followed by a suitable cleavage site, advantageously in a polylinker, for insertion of the nucleic acid to be expressed and, if appropriate, a terminator sequence is positioned behind the polylinker. This sequence is repeated several times, preferably three, four or five times, so that up to five genes can be combined in one construct and introduced into the transgenic plant in order to be expressed. Advantageously, the sequence is repeated up to three times. To express the nucleic acid sequences, the latter are inserted behind the promoter via a suitable cleavage site, for example in the polylinker. Advantageously, each nucleic acid sequence has its own promoter and, if appropriate, its own terminator sequence. Such advantageous constructs are disclosed, for example, in DE 101 02 337 or DE 101 02 338. However, it is also possible to insert a plurality of nucleic acid sequences behind a promoter and, if appropriate, before a terminator sequence. Here, the insertion site, or the sequence, of the inserted nucleic acids in the expression cassette is not of critical importance, that is to say a nucleic acid sequence can be inserted at the first or last position in the cassette without its expression being substantially influenced thereby. Advantageously, different promoters such as, for example, the USP, LegB4 or DC3 promoter, and different terminator sequences can be used in the expression cassette. However, it is also possible to use only one type of promoter in the cassette. This, however, may lead to undesired recombination events.

As described above, the transcription of the genes which have been introduced should advantageously be terminated by suitable terminator sequences at the 3′ end of the biosynthesis genes which have been introduced (behind the stop codon). An example of a sequence which can be used in this context is the OCS 1 terminator sequence. As is the case with the promoters, different terminator sequences should be used for each gene.

As described above, the gene construct can also comprise further genes to be introduced into the organisms. It is possible and advantageous to introduce into the host organisms, and to express therein, regulatory genes such as genes for inductors, repressors or enzymes which, owing to their enzyme activity, engage in the regulation of one or more genes of a biosynthesis pathway. These genes can be of heterologous or of homologous origin. Moreover, further biosynthesis genes of the fatty acid or lipid metabolism can advantageously be present in the nucleic acid construct, or gene construct; however, these genes can also be positioned on one or more further nucleic acid constructs. Biosynthesis genes of the fatty acid or lipid metabolism which are advantageously used is a gene selected from the group consisting of acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferases, fatty acid synthase(s), fatty acid hydroxylase(s), acetyl-coenzyme A carboxylase(s), acyl-coenzyme A oxidase(s), fatty acid desaturase(s), fatty acid acetylenase(s), lipoxygenase(s), triacylglycerol lipase(s), allene oxide synthase(s), hydroperoxide lyase(s) or fatty acid elongase(s) or combinations thereof. Especially advantageous nucleic acid sequences are biosynthesis genes of the fatty acid or lipid metabolism selected from the group of the acyl-CoA:lysophospholipid acyltransferase, Δ4-desaturase, Δ5-desaturase, Δ6-desaturase, Δ9-desaturase, Δ12-desaturase, Δ5-elongase and/or Δ6-elongase.

In this context, the abovementioned nucleic acids or genes can be cloned into expression cassettes, like those mentioned above, in combination with other elongases and desaturases and used for transforming plants with the aid of Agrobacterium.

Here, the regulatory sequences or factors can, as described above, preferably have a positive effect on, and thus enhance, the expression of genes which have been introduced. Thus, enhancement of the regulatory elements can advantageously take place at the transcriptional level by using strong transcription signals such as promoters and/or enhancers. However, an enhanced translation is also possible, for example by improving the stability of the mRNA. In principle, the expression cassettes can be used directly for introduction into the plant or else be introduced into a vector.

These advantageous vectors, preferably expression vectors, comprise the nucleic acids which encode the Δ12-desaturase, Δ6-desaturase, Δ6-elongase, Δ5-desaturases, Δ5-elongase or Δ4-desaturase and which are used in the process, or else a nucleic acid construct which the nucleic acid used either alone or in combination with further biosynthesis genes of the fatty acid or lipid metabolism such as the acyl-CoA:lysophospholipid acyltransferases, Δ4-desaturases, Δ5-desaturases, Δ6-desaturases, Δ9-desaturases, Δ12-desaturases, ω3-desaturases, Δ5-elongases and/or Δ6-elongases. As used in the present context, the term “vector” refers to a nucleic acid molecule which is capable of transporting another nucleic acid to which it is bound. One type of vector is a “plasmid”, a circular double-stranded DNA loop into which additional DNA segments can be ligated. A further type of vector is a viral vector, it being possible for additional DNA segments to be ligated into the viral genome. Certain vectors are capable of autonomous replication in a host cell into which they have been introduced (for example bacterial vectors with bacterial replication origin). Other vectors are advantageously integrated into the genome of a host cell when they are introduced into the host cell, and thus replicate together with the host genome. Moreover, certain vectors can govern the expression of genes with which they are in operable linkage. These vectors are referred to in the present context as “expression vectors”. Usually, expression vectors which are suitable for DNA recombination techniques take the form of plasmids. In the present description, “plasmid” and “vector” can be used exchangeably since the plasmid is the form of vector which is most frequently used. However, the invention is also intended to comprise other forms of expression vectors, such as viral vectors, which exert similar functions. Furthermore, the term “vector” is also intended to comprise other vectors with which the skilled worker is familiar, such as phages, viruses such as SV40, CMV, TMV, transposons, IS elements, phasmids, phagemids, cosmids, linear or circular DNA.

The recombinant expression vectors advantageously used in the process comprise the nucleic acids described below or the above-described gene construct in a form which is suitable for expressing the nucleic acids used in a host cell, which means that the recombinant expression vectors comprise one or more regulatory sequences, selected on the basis of the host cells to be used for the expression, which regulatory sequence(s) is/are linked operably with the nucleic acid sequence to be expressed. In a recombinant expression vector, “linked operably” means that the nucleotide sequence of interest is bound to the regulatory sequence(s) in such a way that the expression of the nucleotide sequence is possible and they are bound to each other in such a way that both sequences carry out the predicted function which is ascribed to the sequence (for example in an in-vitro transcription/translation system, or in a host cell if the vector is introduced into the host cell). The term “regulatory sequence” is intended to comprise promoters, enhancers and other expression control elements (for example polyadenylation signals). These regulatory sequences are described, for example, in Goeddel: Gene Expression Technology: Methods in Enzymology 185, Academic Press, San Diego, Calif. (1990), or see: Gruber and Crosby, in: Methods in Plant Molecular Biology and Biotechnolgy, CRC Press, Boca Raton, Fla., Ed.: Glick and Thompson, Chapter 7, 89-108, including the references cited therein. Regulatory sequences comprise those which govern the constitutive expression of a nucleotide sequence in many types of host cell and those which govern the direct expression of the nucleotide sequence only in specific host cells under specific conditions. The skilled worker knows that the design of the expression vector can depend on factors such as the choice of host cell to be transformed, the expression level of the desired protein and the like.

The recombinant expression vectors used can be designed for the expression of Δ12-desaturases, Δ6-desaturases, Δ6-elongases, Δ5-desaturases, Δ5-elongases and/or Δ4-desaturases in prokaryotic or eukaryotic cells. This is advantageous since intermediate steps of the vector construction are frequently carried out in microorganisms for the sake of simplicity. For example, the Δ112-desaturase, Δ6-desaturase, Δ6-elongase, Δ5-desaturase, Δ5-elongase and/or Δ4-desaturase genes can be expressed in bacterial cells, insect cells (using Baculovirus expression vectors), yeast and other fungal cells (see Romanos, M. A., et al. (1992) “Foreign gene expression in yeast: a review”, Yeast 8:423-488; van den Hondel, C. A. M. J. J., et al. (1991) “Heterologous gene expression in filamentous fungi”, in: More Gene Manipulations in Fungi, J. W. Bennet & L. L. Lasure, Ed., pp. 396-428: Academic Press: San Diego; and van den Hondel, C. A. M. J. J., & Punt, P. J. (1991) “Gene transfer systems and vector development for filamentous fungi, in: Applied Molecular Genetics of Fungi, Peberdy, J. F., et al., Ed., pp. 1-28, Cambridge University Press: Cambridge), algae (Falciatore et al., 1999, Marine Biotechnology. 1, 3:239-251), ciliates of the types: Holotrichia, Peritrichia, Spirotrichia, Suctoria, Tetrahymena, Paramecium, Colpidium, Glaucoma, Platyophrya, Potomacus, Desaturaseudocohnilembus, Euplotes, Engelmaniella and Stylonychia, in particular of the genus Stylonychia lemnae, using vectors in a transformation method as described in WO 98/01572 and, preferably, in cells of multi-celled plants (see Schmidt, R. and Willmitzer, L. (1988) “High efficiency Agrobacterium tumefaciens-mediated transformation of Arabidopsis thaliana leaf and cotyledon explants” Plant Cell Rep.:583-586; Plant Molecular Biology and Biotechnology, C Press, Boca Raton, Fla., Chapter 6/7, pp. 71-119 (1993); F. F. White, B. Jenes et al., Techniques for Gene Transfer, in: Transgenic Plants, Vol. 1, Engineering and Utilization, Ed.: Kung and R. Wu, Academic Press (1993), 128-43; Potrykus, Annu. Rev. Plant Physiol. Plant Molec. Biol. 42 (1991), 205-225 (and references cited therein)). Suitable host cells are furthermore discussed in Goeddel, Gene Expression Technology: Methods in Enzymology 185, Academic Press, San Diego, Calif. (1990). As an alternative, the recombinant expression vector can be transcribed and translated in vitro, for example using T7-promoter regulatory sequences and T7-polymerase.

In most cases, the expression of proteins in prokaryotes involves the use of vectors comprising constitutive or inducible promoters which govern the expression of fusion or nonfusion proteins. Typical fusion expression vectors are, inter alia, pGEX (Pharmacia Biotech Inc; Smith, D. B., and Johnson, K. S. (1988) Gene 67:31-40), pMAL (New England Biolabs, Beverly, Mass.) und pRIT5 (Pharmacia, Piscataway, N.J.), where glutathione S-transferase (GST), maltose-E-binding protein and protein-A, respectively, is fused with the recombinant target protein.

Examples of suitable inducible nonfusion E. coli expression vectors are, inter alia, pTrc (Amann et al. (1988) Gene 69:301-315) and pET 11d (Studier et al., Gene Expression Technology: Methods in Enzymology 185, Academic Press, San Diego, Calif. (1990) 60-89). The target gene expression from the pTrc vector is based on the transcription from a hybrid trp-lac fusion promoter by the host RNA polymerase. The target gene expression from the vector pET 11d is based on the transcription of a T7-gn10-lac fusion promoter, which is mediated by a viral RNA polymerase (T7 gn1), which is coexpressed. This viral polymerase is provided by the host strains BL21 (DE3) or HMS174 (DE3) from a resident λ-prophage which harbors a T7 gn1 gene under the transcriptional control of the lacUV 5 promoter.

Other vectors which are suitable for prokaryotic organisms are known to the skilled worker, these vectors are, for example in E. coli pLG338, pACYC184, the pBR series such as pBR322, the pUC series such as pUC18 or pUC19, the M113 mp series, pKC30, pRep4, pHS1, pHS2, pPLc236, pMBL24, pLG200, pUR290, pIN-III113-B1, λgt11 or pBdCl, in Streptomyces pIJ101, pIJ364, pIJ702 or pIJ361, in Bacillus pUB110, pC194 or pBD214, in Corynebacterium pSΔ77 or pAJ667.

In a further embodiment, the expression vector is a yeast expression vector. Examples for vectors for expression in the yeast S. cerevisiae comprise pYeDesaturasec1 (Baldari et al. (1987) Embo J. 6:229-234), pMFa (Kurjan and Herskowitz (1982) Cell 30:933-943), pJRY 88 (Schultz et al. (1987) Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi [J. W. Bennet & L. L. Lasure, Ed., pp. 396-428: Academic Press: San Diego]. Further suitable yeast vectors are; for example, pAG-1, YEp6, YEp13 or pEMBLYe23.

As an alternative, Δ12-desaturase, Δ6-desaturase, Δ6-elongase, Δ5-desaturase, Δ5-elongase and/or Δ4-desaturase can be expressed in insect cells using Baculovirus vectors. Baculovirus vectors which are available for the expression of proteins in cultured insect cells (for example Sf9 cells) comprise the pAc series (Smith et al. (1983) Mol. Cell. Biol. 3:2156-2165) and the pVL series (Lucklow and Summers (1989) Virology 170:31-39).

The abovementioned vectors are only a small overview over suitable vectors which are possible. Further plasmids are known to the skilled worker and are described, for example, in: Cloning Vectors (Ed. Pouwels, P. H., et al., Elsevier, Amsterdam-New York-Oxford, 1985, ISBN 0 444 904018). For further suitable expression systems for prokaryotic and eukaryotic cells, see the Chapters 16 and 17 in Sambrook, J., Fritsch, E. F., and Maniatis, T., Molecular Cloning: A Laboratory Manual, 2. edition, Cold Spring Harbor Laboratory, Cold Spring Harbor Laboratory Press, Cold Spring Harbor, N.Y., 1989.

In a further embodiment of the process, the Δ12-desaturase, Δ6-desaturase, Δ6-elongase, Δ5-desaturase, Δ5-elongase and/or Δ4-desaturase can be expressed in single-celled plant cells (such as algae), see Falciatore et al., 1999, Marine Biotechnology 1 (3):239-251 and references cited therein, and in plant cells from higher plants (for example spermatophytes such as arable crops). Examples of plant expression vectors comprise those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, p. 15-38.

A plant expression cassette preferably comprises regulatory sequences which are capable of governing the expression of genes in plant cells and which are linked operably so that each sequence can fulfill its function, such as transcriptional termination, for example polyadenylation signals. Preferred polyadenylation signals are those which are derived from Agrobacterium tumefaciens T-DNA, such as gene 3 of the Ti plasmid pTiACH5 (Gielen et al., EMBO J. 3 (1984) 835 et seq.), which is known as octopine synthase, or functional equivalents thereof, but all other terminator sequences which are functionally active in plants are also suitable.

Since plant gene expression is very often not limited to the transcriptional level, a plant expression cassette preferably comprises other sequences which are linked operably, such as translation enhancers, for example the overdrive sequence, which enhances the tobacco mosaic virus 5′-untranslated leader sequence, which increases the protein/RNA ratio (Gallie et al., 1987, Nucl. Acids Research 15:8693-8711).

As described above, the plant gene expression must be linked operably with a suitable promoter which triggers gene expression with the correct timing or in a cell- or tissue-specific manner. Utilizable promoters are constitutive promoters (Benfey et al., EMBO J. 8 (1989) 2195-2202), such as those which are derived from plant viruses, such as 35S CaMV (Franck et al., Cell 21 (1980) 285-294), 19S CaMV (see also U.S. Pat. No. 5,352,605 and WO 84/02913), or plant promoters, such as the promoter of the small Rubisco subunit, which is described in U.S. Pat. No. 4,962,028.

Other preferred sequences for use in operable linkage in plant gene expression cassettes are targeting sequences, which are required for steering the gene product into its corresponding cell compartment (see a review in Kermode, Crit. Rev. Plant Sci. 15, 4 (1996) 285-423 and references cited therein), for example into the vacuole, into the nucleus, all types of plastids, such as amyloplasts, chloroplasts, chromoplasts, the extracellular space, the mitochondria, the endoplasmid reticulum, elaioplasts, peroxisomes and other compartments of plant cells.

As described above, plant gene expression can also be achieved via a chemically inducible promoter (see review in Gatz 1997, Annu. Rev. Plant Physiol. Plant Mol. Biol., 48:89-108). Chemically inducible promoters are particularly suitable when it is desired that the gene expression takes place in a time-specific manner. Examples of such promoters are a salicylic-acid-inducible promoter (WO 95/19443), a tetracyclininducible promoter (Gatz et al. (1992) Plant J. 2, 397-404) and an ethanol-inducible promoter.

Promoters which respond to biotic or abiotic stress conditions are also suitable, for example the pathogen-induced PRP1 gene promoter (Ward et al., Plant. Mol. Biol. 22 (1993) 361-366), the heat-inducible tomato hsp80 promoter (U.S. Pat. No. 5,187,267), the chill-inducible potato alpha-amylase promoter (WO 96/12814) or the wound-inducible pinll promoter (EP-A-0 375 091).

Especially preferred are those promoters which bring about the gene expression in tissues and organs in which the biosynthesis of fatty acids, lipids and oils takes place, in seed cells, such as cells of the endosperm and of the developing embryo. Suitable promoters are the oilseed rape napin gene promoter (U.S. Pat. No. 5,608,152), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legumine B4 promoter (LeB4; Baeumlein et al., 1992, Plant Journal, 2 (2):233-9), and promoters which bring about the seed-specific expression in monocotyledonous plants such as maize, barley, wheat, rye, rice and the like. Suitable noteworthy promoters are the barley lpt2 or lpt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890.

In particular, it may be desired to bring about the multiparallel expression of the Δ12-desaturase, Δ6-desaturase, Δ6-elongase, Δ5-desaturase, Δ5-elongase and/or Δ4-desaturase used in the process. Such expression cassettes can be introduced via the simultaneous transformation of a plurality of individual expression constructs or, preferably, by combining a plurality of expression cassettes on one construct. Also, a plurality of vectors can be transformed with in each case a plurality of expression cassettes and then transferred into the host cell.

Other promoters which are likewise especially suitable are those which bring about a plastid-specific expression, since plastids constitute the compartment in which the precursors and some end products of lipid biosynthesis are synthesized. Suitable promoters, such as the viral RNA polymerase promoter, are described in WO 95/16783 and WO 97/06250, and the cipP promoter from Arabidopsis, described in WO 99/46394.

Vector DNA can be introduced into prokaryotic and eukaryotic cells via conventional transformation or transfection techniques. The terms “transformation” and “transfection”, conjugation and transduction, as used in the present context, are intended to comprise a multiplicity of methods known in the prior art for the introduction of foreign nucleic acid (for example DNA) into a host cell, including calcium phosphate or calcium chloride coprecipitation, DEAE-dextran-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2nd ed., Cold Spring Harbor Laboratory, Cold Spring Harbor Laboratory Press, Cold Spring Harbor, N.Y., 1989) and other laboratory textbooks such as Methods in Molecular Biology, 1995, Vol. 44, Agrobacterium protocols, Ed.: Gartland and Davey, Humana Press, Totowa, N.J.

Host cells which are suitable in principle for taking up the nucleic acid according to the invention, the gene product according to the invention or the vector according to the invention are all prokaryotic or eukaryotic organisms. The host organisms which are advantageously used are microorganisms such as fungi or yeasts, or plant cells, preferably plants or parts thereof. Fungi, yeasts or plants are preferably used, especially preferably plants, very especially preferably plants such as oil crops, which are high in lipid compounds, such as oilseed rape, evening primrose, hemp, thistle, peanut, canola, linseed, soybean, safflower, sunflower, borage, or plants such as maize, wheat, rye, oats, triticale, rice, barley, cotton, cassaya, pepper, Tagetes, Solanacea plants such as potato, tobacco, eggplant and tomato, Vicia species, pea, alfalfa, bushy plants (coffee, cacao, tea), Salix species, trees (oil palm, coconut), and perennial grasses and fodder crops. Especially preferred plants according to the invention are oil crops such as soybean, peanut, oilseed rape, canola, linseed, hemp, evening primrose, sunflower, safflower, trees (oil palm, coconut).

The invention furthermore relates to the nucleic acid sequences which are enumerated hereinbelow and which encode Δ6-desaturases, Δ5-desaturases, Δ4-desaturases or Δ12-desaturases.

Isolated nucleic acid sequences encoding polypeptides with Δ6-desaturase activity, selected from the group consisting of:
a) a nucleic acid sequence with the sequence shown in SEQ ID NO:13,
b) nucleic acid sequences which, as the result of the degeneracy of the genetic code, can be derived from the amino acid sequence shown in SEQ ID NO:14, or
c) derivatives of the nucleic acid sequence shown in SEQ ID NO:13 which encode polypeptides with at least 40% homology at the amino acid level with SEQ ID NO:14 and which have Δ6-desaturase activity.

Isolated nucleic acid sequences encoding polypeptides with Δ5-desaturase activity, selected from the group consisting of:
a) a nucleic acid sequence with the sequence shown in SEQ ID NO:9 or in SEQ ID NO:11,
b) nucleic acid sequences which, as the result of the degeneracy of the genetic code, can be derived from the amino acid sequence shown in SEQ ID NO:10 or in SEQ ID NO:12, or
c) derivatives of the nucleic acid sequence shown in SEQ ID NO:9 or in SEQ ID NO:11 which encode polypeptides with at least 40% homology at the amino acid level with SEQ ID NO:10 or in SEQ ID NO:12 and which have Δ5-desaturase activity.

Isolated nucleic acid sequences encoding polypeptides with Δ4-desaturase activity, selected from the group consisting of:
a) a nucleic acid sequence with the sequence shown in SEQ ID NO:7,
b) nucleic acid sequences which, as the result of the degeneracy of the genetic code, can be derived from the amino acid sequence shown in SEQ ID NO:8, or
c) derivatives of the nucleic acid sequence shown in SEQ ID NO:7 which encode polypeptides with at least 40% homology at the amino acid level with SEQ ID NO:8 and which have Δ6-desaturase activity.

Isolated nucleic acid sequences encoding polypeptides with Δ12-desaturase activity, selected from the group consisting of:
a) a nucleic acid sequence with the sequence shown in SEQ ID NO:15,
b) nucleic acid sequences which, as the result of the degeneracy of the genetic code, can be derived from the amino acid sequence shown in SEQ ID NO:16, or
c) derivatives of the nucleic acid sequence shown in SEQ ID NO:15 which encode polypeptides with at least 50% homology at the amino acid level with SEQ ID NO:16 and which have Δ12-desaturase activity.

The abovementioned nucleic acids according to the invention are derived from organisms such as nonhuman animals, ciliates, fungi, plants such as algae or dinoflagellates which are capable of synthesizing PUFAs.

The isolated abovementioned nucleic acid sequences are advantageously derived from the order Salmoniformes, the diatom genera Thalassiosira or Crypthecodinium, or from the family of the Prasinophyceae, such as the genus Ostreococcus or Pythiaceae, such as the genus Phytophtora.

As described above, the inventive subject matter further includes isolated nucleic acid sequence which encode polypeptides with Δ112-desaturases, Δ4-desaturases, Δ5-desaturases and Δ6-desaturases, where the Δ12-desaturases, Δ4-desaturases, Δ5-desaturases or Δ6-desaturases encoded by these nucleic acid sequences convert C₁₈-, C₂₀- and C₂₂-fatty acids with one, two, three, four or five double bonds and advantageously polyunsaturated C₁₈-fatty acids with one, two or three double bonds such as C18:1^{Δ9}, C18:2^{Δ9,12} or C18:3^{Δ9,12,15}, polyunsaturated C₂₀-fatty acids with three or four double bonds such as C20:3^{Δ8,11,14} or C20:4^{Δ8,11,14,17} or polyunsaturated C₂₂-fatty acids with four or five double bonds such as C22:4^{Δ7,10,13,16} or C22:5^{Δ7,10,13,16,19}. The fatty acids are advantageously desaturated in the phospholipids or CoA-fatty acid esters, advantageously in the CoA-fatty acid esters.

In an advantageous embodiment, the term “nucleic acid (molecule)” as used in the present context additionally comprises the untranslated sequence at the 3′ and at the 5′ end of the coding gene region: at least 500, preferably 200, especially preferably 100 nucleotides of the sequence upstream of the 5′ end of the coding region and at least 100, preferably 50, especially preferably 20 nucleotides of the sequence downstream of the 3′ end of the coding gene region. An “isolated” nucleic acid molecule is separated from other nucleic acid molecules which are present in the natural source of the nucleic acid. An “isolated” nucleic acid preferably has no sequences which naturally flank the nucleic acid in the genomic DNA of the organism from which the nucleic acid is derived (for example sequences which are located at the 5′ and 3′ ends of the nucleic acid). In various embodiments, the isolated Δ12-desaturase, Δ6-desaturase, Δ6-elongase, Δ5-desaturase, Δ5-elongase or Δ4-desaturase molecule can comprise for example fewer than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived.

The nucleic acid molecules used in the process, for example a nucleic acid molecule with a nucleotide sequence of SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 or of a part thereof can be isolated using molecular-biological standard techniques and the sequence information provided herein. Also, for example a homologous sequence or homologous, conserved sequence regions can be identified at the DNA or amino acid level with the aid of comparative algorithms. They can be used as hybridization probe and standard hybridization techniques (such as, for example, those described in Sambrook et al., Molecular Cloning: A Laboratory Manual. 2nd ed., Cold Spring Harbor Laboratory, Cold Spring Harbor Laboratory Press, Cold Spring Harbor, N.Y., 1989) for isolating further nucleic acid sequences which can be used in the process. Moreover, a nucleic acid molecule comprising a complete sequence of SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 or a part thereof can be isolated by polymerase chain reaction, where oligonucleotide primers which are on the basis of this sequence or on parts thereof are used (for example a nucleic acid molecule comprising the complete sequence or part thereof can be isolated by polymerase chain reaction using oligonucleotide primers which have been generated based on this same sequence). For example, mRNA can be isolated from cells (for example by means of the guanidinium thiocyanate extraction method of Chirgwin et al. (1979) Biochemistry 18:5294-5299) and cDNA by means of reverse transcriptase (for example Moloney MLV reverse transcriptase, available from Gibco/BRL, Bethesda, Md., or AMV reverse transcriptase, available from Seikagaku America, Inc., St. Petersburg, Fla.). Synthetic oligonucleotide primers for the amplification by means of polymerase chain reaction can be generated based on one of the sequences shown in SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 or with the aid of the amino acid sequences detailed in SEQ ID NO: 2, SEQ ID NO: 4, SEQ ID NO:6, SEQ ID NO: 8, SEQ ID NO: 10, SEQ ID NO: 12, SEQ ID NO: 14 or SEQ ID NO: 16. A nucleic acid according to the invention can be amplified by standard PCR amplification techniques using cDNA or, alternatively, genomic DNA as template and suitable oligonucleotide primers. The nucleic acid thus amplified can be cloned into a suitable vector and characterized by means of DNA sequence analysis. Oligonucleotides which correspond to a desaturase nucleotide sequence can be generated by standard synthetic methods, for example using an automatic DNA synthesizer.

Homologs of the Δ12-desaturase, Δ6-desaturase, Δ6-elongase, Δ5-desaturase, Δ5-elongase or Δ4-desaturase nucleic acid sequences with the sequence SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 means, for example, allelic variants with at least approximately 40 or 50%, preferably at least approximately 60 or 70%, more preferably at least approximately 70 or 80%, 90% or 95% and even more preferably at least approximately 85%, 86%, 87%, 88%, 89%, 90%, 91%, 92%, 93%, 94%, 95%, 96%, 97%, 98%, 99% or more identity or homology with a nucleotide sequences shown in SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 or its homologs, derivatives or analogs or parts thereof. Furthermore, isolated nucleic acid molecules of a nucleotide sequence which hybridize with one of the nucleotide sequences shown in SEQ ID NO: 1, SEQ ID. NO: 3, SEQ ID NO:5, SEQ ID NO: 7, SEQ ID NO: 9, SEQ ID NO: 11, SEQ ID NO: 13 or SEQ ID NO: 15 or with a part thereof, for example hybridized under stringent conditions. A part thereof is understood as meaning, in accordance with the invention, that at least 25 base pairs (=bp), 50 bp, 75 bp, 100 bp, 125 bp or 150 bp, preferably at least 175 bp, 200 bp, 225 bp, 250 bp, 275 bp or 300 bp, especially preferably 350 bp, 400 bp, 450 bp, 500 bp or more base pairs are used for the hybridization. It is also possible and advantageous to use the full sequence. Allelic variants comprise in particular functional variants which can be obtained by deletion, insertion or substitution of nucleotides from/into the sequence detailed in SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15, it being intended, however, that the enzyme activity of the resulting proteins which are synthesized is advantageously retained for the insertion of one or more genes. Proteins which retain the enzymatic activity of Δ12-desaturase, Δ6-desaturase, Δ6-elongase, Δ5-desaturase, Δ5-elongase or Δ4-desaturase, i.e. whose activity is essentially not reduced, means proteins with at least 10%, preferably 20%, especially preferably 30%, very especially preferably 40% of the original enzyme activity in comparison with the protein encoded by SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15. The homology was calculated over the entire amino acid or nucleic acid sequence region. The skilled worker has available a series of programs which are based on various algorithms for the comparison of various sequences. Here, the algorithms of Needleman and Wunsch or Smith and Waterman give particularly reliable results. The program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970) and Smith and Waterman (Adv. Appl. Math. 2; 482-489 (1981)], which are part of the GCG software packet [Genetics Computer Group, 575 Science Drive, Madison, Wis., USA 53711 (1991)], were used for the sequence alignment. The sequence homology values which are indicated above as a percentage were determined over the entire sequence region using the program GAP and the following settings: Gap Weight: 50, Length Weight: 3, Average Match: 10.000 and Average Mismatch: 0.000. Unless otherwise specified, these settings were always used as standard settings for the sequence alignments.

Homologs of SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 means for example also bacterial, fungal and plant homologs, truncated sequences, single-stranded DNA or RNA of the coding and noncoding DNA sequence.

Homologs of SEQ ID NO: 1, SEQ ID NO: 3, SEQ ID NO:5, SEQ ID NO: 7, SEQ ID NO: 9, SEQ ID NO: 11, SEQ ID NO: 13 or SEQ ID NO: 15 also means derivatives such as, for example, promoter variants. The promoters upstream of the nucleotide sequences detailed can be modified by one or more nucleotide exchanges, by insertion(s) and/or deletion(s) without the functionality or activity of the promoters being adversely affected, however. It is furthermore possible that the modification of the promoter sequence enhances their activity or that they are replaced entirely by more active promoters, including those from heterologous organisms.

The abovementioned nucleic acids and protein molecules with Δ12-desaturase, Δ6-desaturase, Δ6-elongase, Δ5-desaturase, Δ5-elongase and/or Δ4-desaturase activity which are involved in the metabolism of lipids and fatty acids, PUFA cofactors and enzymes or in the transport of lipophilic compounds across membranes are used in the process according to the invention for the modulation of the production of PUFAs in transgenic organisms, advantageously in plants, such as maize, wheat, rye, oats, triticale, rice, barley, soybean, peanut, cotton, Linum species such as linseed or flax, Brassica species such as oilseed rape, canola and turnip rape, pepper, sunflower, borage, evening primrose and Tagetes, Solanaceae plants such as potato, tobacco, eggplant and tomato, Vicia species, pea, cassaya, alfalfa, bushy plants (coffee, cacao, tea), Salix species, trees (oil palm, coconut) and perennial grasses and fodder crops, either directly (for example when the overexpression or optimization of a fatty acid biosynthesis protein has a direct effect on the yield, production and/or production efficiency of the fatty acid from modified organisms) and/or can have an indirect effect which nevertheless leads to an enhanced yield, production and/or production efficiency of the PUFAs or a reduction of undesired compounds (for example when the modulation of the metabolism of lipids and fatty acids, cofactors and enzymes lead to modifications of the yield, production and/or production efficiency or the composition of the desired compounds within the cells, which, in turn, can affect the production of one or more fatty acids).

The combination of various precursor molecules and biosynthesis enzymes leads to the production of various fatty acid molecules, which has a decisive effect on lipid composition, since polyunsaturated fatty acids (=PUFAs) are not only incorporated into triacylglycerol but also into membrane lipids.

Brassicaceae, Boraginaceae, Primulaceae, or Linaceae are particularly suitable for the production of PUFAs, for example stearidonic acid, eicosapentaenoic acid and docosahexaenoic acid. Linseed (Linum usitatissimum) is especially advantageously suitable for the production of PUFAs with the nucleic acid sequences according to the invention, advantageously, as described, in combination with further desaturases and elongases.

Lipid synthesis can be divided into two sections: the synthesis of fatty acids and their binding to sn-glycerol-3-phosphate, and the addition or modification of a polar head group. Usual lipids which are used in membranes comprise phospholipids, glycolipids, sphingolipids and phosphoglycerides. Fatty acid synthesis starts with the conversion of acetyl-CoA into malonyl-CoA by acetyl-CoA carboxylase or into acetyl-ACP by acetyl transacylase. After a condensation reaction, these two product molecules together form acetoacetyl-ACP, which is converted via a series of condensation, reduction and dehydratation reactions so that a saturated fatty acid molecule with the desired chain length is obtained. The production of the unsaturated fatty acids from these molecules is catalyzed by specific desaturases, either aerobically by means of molecular oxygen or anaerobically (regarding the fatty acid synthesis in microorganisms, see F. C. Neidhardt et al. (1996) E. coli and Salmonella. ASM Press: Washington, D.C., pp. 612-636 and references cited therein; Lengeler et al. (Ed.) (1999) Biology of Procaryotes; Thieme: Stuttgart, N.Y., and the references therein, and Magnuson, K., et al. (1993) Microbiological Reviews 57:522-542 and the references therein). To undergo the further elongation steps, the resulting phospholipid-bound fatty acids must be returned to the fatty acid CoA ester pool. This is made possible by acyl-CoA:lysophospholipid acyltransferases. Moreover, these enzymes are capable of transferring the elongated fatty acids from the CoA esters back to the phospholipids. If appropriate, this reaction sequence can be followed repeatedly.

Examples of precursors for the biosynthesis of PUFAs are oleic acid, linoleic acid and linolenic acid. These C₁₈-carbon fatty acids must be elongated to C₂₀ and C₂₂ in order to obtain fatty acids of the eicosa and docosa chain type. With the aid of the desaturases used in the process, such as the Δ12-, Δ4-, Δ5- and Δ6-desaturases and/or Δ5-, Δ6-elongases, arachidonic acid, eicosapentaenoic acid, docosapentaenoic acid or docosahexaenoic acid, advantageously eicosapentaenoic acid and/or docosahexaenoic acid, can be produced and subsequently employed in various applications regarding foodstuffs, feedstuffs, cosmetics or pharmaceuticals. C₂₀- and/or C₂₂-fatty acids with at least two, advantageously at least three, four, five or six, double bonds in the fatty acid molecule, preferably C₂₀- or C₂₂-fatty acids with advantageously four, five or six double bonds in the fatty acid molecule, can be prepared using the abovementioned enzymes. Desaturation may take place before or after elongation of the fatty acid in question. This is why the products of the desaturase activities and the further desaturation and elongation steps which are possible result in preferred PUFAs with a higher degree of desaturation, including a further elongation from C₂₀- to C₂₂-fatty acids, to fatty acids such as γ-linolenic acid, dihomo-γ-linolenic acid, arachidonic acid, stearidonic acid, eicosatetraenoic acid or eicosapentaenoic acid. Substrates of the desaturases and elongases used in the process according to the invention are C₁₆-, C₁₈- or C₂₀-fatty acids such as, for example, linoleic acid, γ-linolenic acid, α-linolenic acid, dihomo-γ-linolenic acid, eicosatetraenoic acid or stearidonic acid. Preferred substrates are linoleic acid, γ-linolenic acid and/or α-linolenic acid, dihomo-γ-linolenic acid or arachidonic acid, eicosatetraenoic acid or eicosapentaenoic acid. The synthesized C₂₀- or C₂₂-fatty acids with at least two, three, four, five or six double bonds in the fatty acids are obtained in the process according to the invention in the form of the free fatty acid or in the form of their esters, for example in the form of their glycerides.

The term “glyceride” is understood as meaning glycerol esterified with one, two or three carboxyl radicals (mono-, di- or triglyceride). “Glyceride” is also understood as meaning a mixture of various glycerides. The glyceride or glyceride mixture may comprise further additions, for example free fatty acids, antioxidants, proteins, carbohydrates, vitamins and/or other substances.

For the purposes of the invention, a “glyceride” is furthermore understood as meaning glycerol, derivatives. In addition to the above-described fatty acid glycerides, these also include glycerophospholipids and glyceroglycolipids. Preferred examples which may be mentioned in this context are the glycerophospholipids such as lecithin (phosphatidylcholine), cardiolipin, phosphatidylglycerol, phosphatidylserine and alkylacylglycerophospholipids.

Furthermore, fatty acids must subsequently be translocated to various modification sites and incorporated into the triacylglycerol storage lipid. A further important step in lipid synthesis is the transfer of fatty acids to the polar head groups, for example by glycerol fatty acid acyltransferase (see Frentzen, 1998, Lipid, 100(4-5):161-166).

Publications on plant fatty acid biosynthesis and on the desaturation, the lipid metabolism and the membrane transport of lipidic compounds, on beta-oxidation, fatty acid modification and cofactors, triacylglycerol storage and triacylglycerol assembly, including the references therein, see the following papers: Kinney, 1997, Genetic Engineering, Ed.: J K Setlow, 19:149-166; Ohlrogge and Browse, 1995, Plant Cell 7:957-970; Shanklin and Cahoon, 1998, Annu. Rev. Plant Physiol. Plant. Mol. Biol. 49:611-641; Voelker, 1996, Genetic Engineering, Ed.: J K Setlow, 18:111-13; Gerhardt, 1992, Prog. Lipid R. 31:397-417; Guhnemann-Schafer & Kindl, 1995, Biochim. Biophys Acta 1256:181-186; Kunau et al., 1995, Prog. Lipid Res. 34:267-342; Stymne et al., 1993, in: Biochemistry and Molecular Biology of Membrane and Storage Lipids of Plants, Ed.: Murata and Somerville, Rockville, American Society of Plant Physiologists, 150-158, Murphy & Ross 1998, Plant Journal. 13(1):1-16.

The PUFAs produced in the process comprise a group of molecules which higher animals are no longer capable of synthesizing and must therefore take up, or which higher animals are no longer capable of synthesizing themselves in sufficient quantity and must therefore take up additional quantities, although they can be synthesized readily by other organisms such as bacteria; for example, cats are no longer capable of synthesizing arachidonic acid.

Phospholipids for the purposes of the invention are understood as meaning phosphatidylcholine, phosphatidylethanolamine, phosphatidylserine, phosphatidylglycerol and/or phosphatidylinositol, advantageously phosphatidylcholine. The terms production or productivity are known in the art and encompass the concentration of the fermentation product (compounds of the formula I) which is formed within a specific period of time and in a specific fermentation volume (for example kg of product per hour per liter). It also comprises the productivity within a plant cell or a plant, that is to say the content of the desired fatty acids produced in the process relative to the content of all fatty acids in this cell or plant. The term production efficiency comprises the time required for obtaining a specific production quantity (for example the time required by the cell to establish a certain throughput rate of a fine chemical). The term yield or product/carbon yield is known in the art and comprises the efficiency of the conversion of the carbon source into the product (i.e. the fine chemical). This is usually expressed for example as kg of product per kg of carbon source. By increasing the yield or production of the compound, the amount of the molecules obtained of this compound, or of the suitable molecules of this compound obtained, in a specific culture quantity over a specified period of time is increased. The terms biosynthesis or biosynthetic pathway are known in the art and comprise the synthesis of a compound, preferably an organic compound, by a cell from intermediates, for example in a multi-step and strongly regulated process. The terms catabolism or catabolic pathway are known in the art and comprise the cleavage of a compound, preferably of an organic compound, by a cell to give catabolites (in more general terms, smaller or less complex molecules), for example in a multi-step and strongly regulated process. The term metabolism is known in the art and comprises the totality of the biochemical reactions which take place in an organism. The metabolism of a certain compound (for example the metabolism of a fatty acid) thus comprises the totality of the biosynthetic pathways, modification pathways and catabolic pathways of this compound in the cell which relate to this compound.

In a further embodiment, derivatives of the nucleic acid molecule according to the invention represented in SEQ ID NO: 7, SEQ ID NO: 9, SEQ ID NO: 11, SEQ ID NO: 13 or SEQ ID NO: 15 encode proteins with at least 40%, advantageously approximately 50 or 60%, advantageously at least approximately 60 or 70% and more preferably at least approximately 70 or 80%, 80 to 90%, 90 to 95% and most preferably at least approximately 96%, 97%, 98%, 99% or more homology (=identity) with a complete amino acid sequence of SEQ ID NO: 8, SEQ ID NO: 10, SEQ ID NO: 12, SEQ ID NO: 14 or SEQ ID NO: 16. The homology was calculated over the entire amino acid or nucleic acid sequence region. The program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970) and Smith and Waterman (Adv. Appl. Math. 2; 482-489 (1981)], which are part of the GCG software packet [Genetics Computer Group, 575 Science Drive, Madison, Wis., USA 53711 (1991)], were used for the sequence alignments. The sequence homology values which are indicated above as a percentage were determined over the entire sequence region using the program BestFit and the following settings: Gap Weight: 50, Length Weight: 3, Average Match: 10.000 and Average Mismatch: 0.000. Unless otherwise specified, these settings were always used as standard settings for the sequence alignments.

Moreover, the invention comprises nucleic acid molecules which differ from one of the nucleotide sequences shown in SEQ ID NO: 7, SEQ ID NO: 9, SEQ ID NO: 11, SEQ ID NO: 13 or SEQ ID NO: 15 (and parts thereof) owing to the degeneracy of the genetic code and which thus encode the same Δ112-desaturase, Δ6-desaturase, Δ5-desaturase or Δ4-desaturase as those encoded by the nucleotide sequences shown in SEQ ID NO: 7, SEQ ID NO: 9, SEQ ID NO: 11, SEQ ID NO: 13 or SEQ ID NO: 15.

In addition to the Δ12-desaturases, Δ6-desaturases, Δ5-desaturases or Δ4-desaturases shown in SEQ ID NO: 7, SEQ ID NO: 9, SEQ ID NO: 11, SEQ ID NO: 13 or SEQ ID NO: 15, the skilled worker will recognize that DNA sequence polymorphisms which lead to changes in the amino acid sequences of the Δ12-desaturase, Δ6-desaturase, Δ5-desaturase and/or Δ4-desaturase may exist within a population. These genetic polymorphisms in the Δ12-desaturase, Δ6-desaturase, Δ5-desaturase and/or Δ4-desaturase gene may exist between individuals within a population owing to natural variation. These natural variants usually bring about a variance of 1 to 5% in the nucleotide sequence of the Δ12-desaturase, Δ6-desaturase, Δ5-desaturase and/or Δ4-desaturase gene. Each and every one of these nucleotide variations and resulting amino acid polymorphisms in the Δ12-desaturase, Δ6-desaturase, Δ5-desaturase and/or Δ4-desaturase which are the result of natural variation and do not modify the functional activity are to be encompassed by the invention.

Owing to their homology to the Δ12-desaturase, Δ5-elongase, Δ6-desaturase, Δ5-desaturase, Δ4-desaturase and/or Δ6-elongase nucleic acids disclosed here, nucleic acid molecules which are advantageous for the process according to the invention can be isolated following standard hybridization techniques under stringent hybridization conditions, using the sequences or part thereof as hybridization probe. In this context it is possible, for example, to use isolated nucleic acid molecules which are at least 15 nucleotides in length and which hybridize under stringent conditions with the nucleic acid molecules which comprise a nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15. Nucleic acids with at least 25, 50, 100, 250 or more nucleotides can also be used. The “hybridizes under stringent conditions” as used in the present context is intended to describe hybridization and washing conditions under which nucleotide sequences with at least 60% homology to one another usually remain hybridized with one another. Conditions are preferably such that sequences with at least approximately 65%, preferably at least approximately 70% and especially preferably at least 75% or more homology to one another usually remain hybridized to one another. These stringent conditions are known to the skilled worker and described, for example, in Current Protocols in Molecular Biology, John Wiley & Sons, N.Y. (1989), 6.3.1-6.3.6. A preferred nonlimiting example of stringent hybridization conditions is hybridizations in 6× sodium chloride/sodium citrate (=SSC) at approximately 45° C., followed by one or more washing steps in 0.2×SSC, 0.1% SDS at 50 to 65° C. The skilled worker knows that these hybridization conditions differ depending on the type of nucleic acid and, for example when organic solvents are present, regarding temperature and buffer concentration. Under “standard hybridization conditions”, for example, the temperature is, depending on the type of nucleic acid, between 42° C. and 58° C. in aqueous buffer with a concentration of 0.1 to 5×SSC (pH 7.2). If an organic solvent, for example 50% formamide, is present in the abovementioned buffer, the temperature under standard conditions is approximately 42° C. The hybridization conditions for DNA:DNA hybrids, for example, are 0.1×SSC and 20° C. to 45° C., preferably 30° C. to 45° C. The hybridization conditions for DNA:RNA hybrids are, for example, 0.1×SSC and 30° C. to 55° C., preferably 45° C. to 55° C. The abovementioned hybridization temperatures are determined by way of example for a nucleic acid with approximately 100 bp (=base pairs) in length and with a G+C content of 50% in the absence of formamide. The skilled worker knows how to determine the required hybridization conditions on the basis of the abovementioned textbooks or textbooks such as Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford.

In order to determine the percentage of homology (=identity) of two amino acid sequences (for example one of the sequences of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16) or of two nucleic acids (for example SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15), the sequences are written one under the other for an optimal comparison (for example, gaps may be introduced into the sequence of a protein or of a nucleic acid in order to generate an optimal alignment with the other protein or the other nucleic acid). Then, the amino acid residues or nucleotides at the corresponding amino acid positions or nucleotide positions are compared. If a position in a sequence is occupied by the same amino acid residue or the same nucleotide as the corresponding position in the other sequence, then the molecules are homologous at this position (i.e. amino acid or nucleic acid “homology” as used in the present context corresponds to amino acid or nucleic acid “identity”). The percentage of homology between the two sequences is a function of the number of identical positions which the sequences share (i.e. % homology=number of identical positions/total number of positions×100). The terms homology and identity are therefore to be considered as synonymous. The programs and algorithms used are those described above.

An isolated nucleic acid molecule which encodes a Δ12-desaturase, Δ6-desaturase, Δ5-desaturase, Δ4-desaturase, Δ5-elongase and/or Δ6-elongase which is homologous to a protein sequence of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 can be generated by introducing one or more nucleotide substitutions, additions or deletions in/into a nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 so that one or more amino acid substitutions, additions or deletions are introduced in/into the protein which is encoded. Mutations in one of the sequences of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 can be introduced by standard techniques such as site-specific mutagenesis and PCR-mediated mutagenesis. It is preferred to generate conservative amino acid substitutions in one or more of the predicted nonessential amino acid residues. In a “conservative amino acid substitution”, the amino acid residue is replaced by an amino acid residue with a similar side chain. Families of amino acid residues with similar side chains have been defined in the art. These families comprise amino acids with basic side chains (for example lysine, arginine, histidine), acidic side chains (for example aspartic acid, glutamic acid), uncharged polar side chains (for example glycine, asparagine, glutamine, serine, threonine, tyrosine, cysteine), unpolar side chains (for example alanine, valine, leucine, isoleucine, proline, phenylalanine, methionine, tryptophan), beta-branched side chains (for example threonine, valine, isoleucine) and aromatic side chains (for example tyrosine, phenylalanine, tryptophan, histidine). A predicted nonessential amino acid residue in a Δ12-desaturase, Δ6-desaturase, Δ5-desaturase, Δ4-desaturase, Δ5-elongase or Δ6-elongase is thus preferably replaced by another amino acid residue from the same family of side chains. In another embodiment, the mutations can, alternatively, be introduced randomly over all or part of the sequence encoding the Δ12-desaturase, Δ6-desaturase, Δ5-desaturase, Δ4-desaturase, Δ5-elongase or Δ6-elongase, for example by saturation mutagenesis, and the resulting mutants can be screened by recombinant expression for the herein-described Δ12-desaturase, Δ6-desaturase, Δ5-desaturase, Δ4-desaturase, Δ5-elongase or Δ6-elongase activity in order to identify mutants which have retained the Δ12-desaturase, Δ6-desaturase, Δ5-desaturase, Δ4-desaturase, Δ5-elongase or Δ6-elongase activity. Following the mutagenesis of one of the sequences SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, the protein which is encoded can be expressed recombinantly, and the activity of the protein can be determined, for example using the tests described in the present text.

The invention furthermore relates to transgenic nonhuman organisms which comprise the nucleic acids SEQ ID NO: 7, SEQ ID NO: 9, SEQ ID NO: 11, SEQ ID NO: 13 or SEQ ID NO: 15 according to the invention or a gene construct or a vector which comprise these nucleic acid sequences according to the invention. The nonhuman organism is advantageously a microorganism, a nonhuman animal or a plant, especially preferably a plant.

The present invention is illustrated in greater detail by the examples which follow, which are not to be construed as limiting. The content of all of the references, patent applications, patents and published patent applications cited in the present patent application is herewith incorporated by reference.

### EXAMPLES

### Example 1

### General Cloning Methods

The cloning methods such as, for example, restriction cleavages, agarose gel electrophoresis, purification of DNA fragments, transfer of nucleic acids to nitrocellulose and nylon membranes, linkage of DNA fragments, transformation of Escherichia coli cells, bacterial cultures and the sequence analysis of recombinant DNA were carried out as described by Sambrook et al. (1989) (Cold Spring Harbor Laboratory Press: ISBN 0-87969-309-6).

### Example 2

### Sequence Analysis of Recombinant DNA

Recombinant DNA molecules were sequenced with an ABI laser fluorescence DNA sequencer by the method of Sanger (Sanger et al. (1977) Proc. Natl. Acad. Sci. USA 74, 5463-5467). Fragments obtained by polymerase chain reaction were sequenced and verified to avoid polymerase errors in constructs to be expressed.

### Example 3

### Lipid Extraction from Yeasts and Seeds

The effect of the genetic modification in plants, fungi, algae, ciliates or on the production of a desired compound (such as a fatty acid) can be determined by growing the modified microorganisms or the modified plant under suitable conditions (such as those described above) and analyzing the medium and/or the cellular components for the elevated production of the desired product (i.e. of the lipids or a fatty acid). These analytical techniques are known to the skilled worker and comprise spectroscopy, thin-layer chromatography, various types of staining methods, enzymatic and microbiological methods and analytical chromatography such as high-performance liquid chromatography (see, for example, Ullman, Encyclopedia of Industrial Chemistry, Vol. A2, p. 89-90 and p. 443-613, VCH: Weinheim (1985); Fallon, A., et al., (1987) “Applications of HPLC in Biochemistry” in: Laboratory Techniques in Biochemistry and Molecular Biology, Vol. 17; Rehm et al. (1993) Biotechnology, Vol. 3, Chapter III: “Product recovery and purification”, p. 469-714, VCH: Weinheim; Belter, P. A., et al. (1988) Bioseparations: downstream processing for Biotechnology, John Wiley and Sons; Kennedy, J. F., and Cabral, J. M. S. (1992) Recovery processes for biological Materials, John Wiley and Sons; Shaeiwitz, J. A., and Henry, J. D. (1988) Biochemical Separations, in: Ullmann's Encyclopedia of Industrial Chemistry, Vol. B3; Chapter 11, p. 1-27, VCH: Weinheim; and Dechow, F. J. (1989) Separation and purification techniques in biotechnology, Noyes Publications).

In addition to the abovementioned processes, plant lipids are extracted from plant material as described by Cahoon et al. (1999) Proc. Natl. Acad. Sci. USA 96 (22):12935-12940 and Browse et al. (1986) Analytic Biochemistry 152:141-145. The qualitative and quantitative analysis of lipids or fatty acids is described by Christie, William W., Advances in Lipid Methodology, Ayr/Scotland: Oily Press (Oily Press Lipid Library; 2); Christie, William W., Gas Chromatography and Lipids. A Practical Guide—Ayr, Scotland: Oily Press, 1989, Repr. 1992, IX, 307 pp. (Oily Press Lipid Library; 1); “Progress in Lipid Research”, Oxford: Pergamon Press, 1 (1952)-16 (1977) under the title: Progress in the Chemistry of Fats and Other Lipids CODEN.

In addition to measuring the end product of the fermentation, it is also possible to analyze other components of the metabolic pathways which are used for the production of the desired compound, such as intermediates and by-products, in order to determine the overall production efficiency of the compound. The analytical methods comprise measuring the amount of nutrients in the medium (for example sugars, hydrocarbons, nitrogen sources, phosphate and other ions), measuring the biomass composition and the growth, analyzing the production of conventional metabolites of biosynthetic pathways and measuring gases which are generated during the fermentation. Standard methods for these measurements are described in Applied Microbial Physiology; A Practical Approach, P. M. Rhodes and P. F. Stanbury, Ed., IRL Press, p. 103-129; 131-163 and 165-192 (ISBN: 0199635773) and references cited therein.

One example is the analysis of fatty acids (abbreviations: FAME, fatty acid methyl ester; GC-MS, gas liquid chromatography/mass spectrometry; TAG, triacylglycerol; TLC, thin-layer chromatography).

The unambiguous detection for the presence of fatty acid products can be obtained by analyzing recombinant organisms using standard analytical methods: GC, GC-MS or TLC, as described on several occasions by Christie and the references therein (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometry methods], Lipide 33:343-353).

The material to be analyzed can be disrupted by sonication, grinding in a glass mill, liquid nitrogen and grinding or via other applicable methods. After disruption, the material must be centrifuged. The sediment is resuspended in distilled water, heated for 10 minutes at 100° C., cooled on ice and recentrifuged, followed by extraction for one hour at 90° C. in 0.5 M sulfuric acid in methanol with 2% dimethoxypropane, which leads to hydrolyzed oil and lipid compounds, which give transmethylated lipids. These fatty acid methyl esters are extracted in petroleum ether and finally subjected to a GC analysis using a capillary column (Chrompack, WCOT Fused Silica, CP-Wax-52 CB, 25 μm, 0.32 mm) at a temperature gradient of between 170° C. and 240° C. for 20 minutes and 5 minutes at 240° C. The identity of the resulting fatty acid methyl esters must be defined using standards which are available from commercial sources (i.e. Sigma).

Plant material is initially homogenized mechanically by comminuting in a pestle and mortar to make it more amenable to extraction.

This is followed by heating at 10° C. for 10 minutes and, after cooling on ice, by resedimentation. The cell sediment is hydrolyzed for one hour at 90° C. with 1 M methanolic sulfuric acid and 2% dimethoxypropane, and the lipids are transmethylated. The resulting fatty acid methyl esters (FAMEs) are extracted in petroleum ether. The extracted FAMEs are analyzed by gas liquid chromatography using a capillary column (Chrompack, WCOT Fused Silica, CP-Wax-52 CB, 25 m, 0.32 mm) and a temperature gradient of from 170° C. to 240° C. in 20 minutes and 5 minutes at 240° C. The identity of the fatty acid methyl esters is confirmed by comparison with corresponding FAME standards (Sigma). The identity and position of the double bond can be analyzed further by suitable chemical derivatization of the FAME mixtures, for example to give 4,4-dimethoxyoxazoline derivatives (Christie, 1998) by means of GC-MS.

### Example 4

### Cloning Genes from Ostreococcus tauri

By searching for conserved regions in the protein sequences in elongase genes, it was possible to identify two sequences with corresponding motifs in an Ostreococcus tauri sequence database (genomic sequences). The sequences are the following

| Name of gene | SEQ ID | Amino acids |
|---|---|---|
| OtELO1, (Δ5-elongase) | SEQ ID NO: 1 | 300 |
| OtELO2, (Δ6-elongase) | SEQ ID NO: 5 | 292 |

OtElo1 has the highest similarity with a Danio rerio elongase (GenBank AAN77156; approx. 26% identity), while OtElo2 has the greatest similarity with the Physcomitrella Elo (PSE) [approx. 36% identity] (alignments were carried out using the tBLASTn algorithm (Altschul et al., J. Mol. Biol. 1990, 215: 403-410).

The cloning procedure was carried out as follows:

40 ml of an Ostreococcus tauri culture in the stationary phase were spun down and the pellet was resuspended in 100 μl of double-distilled water and stored at −20° C. The relevant genomic DNAs were amplified based on the PCR method. The corresponding primer pairs were selected in such a way that they contained the yeast consensus sequence for highly efficient translation (Kozak, Cell 1986, 44:283-292) next to the start codon. The amplification of the OtElo-DNAs was carried out using in each case 1 μl of defrosted cells, 200 μM dNTPs, 2.5 U Taq polymerase and 100 μmol of each primer in a total volume of 50 μl. The conditions for the PCR were as follows: first denaturation at 95° C. for 5 minutes, followed by 30 cycles at 94° C. for 30 seconds, 55° C. for 1 minute and 72° C. for 2 minutes, and a final elongation step at 72° C. for 10 minutes.

### Example 5

### Cloning of Expression Plasmids for Heterologous Expression in Yeasts

To characterize the function of the Ostreococcus tauri elongases, the open reading frames of the DNAs in question were cloned downstream of the galactose-inducible GALL promoter of pYES2.1N/5-His-TOPO (Invitrogen), giving rise to pOTE1 and pOTE2.

The Saccharomyces cerevisiae strain 334 was transformed with the vector pOTE1 or pOTE2, respectively, by electroporation (1500 V). A yeast which was transformed with the blank vector pYES2 was used as control. The transformed yeasts were selected on complete minimal dropout uracil medium (CMdum) agar plates supplemented with 2% glucose. After the selection, in each case three transformants were selected for the further functional expression.

To express the Ot elongases, precultures consisting of in each case 5 ml of CMdum dropout uracil liquid medium supplemented with 2% (w/v) raffinose were initially inoculated with the selected transformants and incubated for 2 days at 30° C. and 200 rpm. Then, 5 ml of CMdum (dropout uracil) liquid medium supplemented with 2% raffinose and 300 μM various fatty acids were inoculated with the precultures to an OD₆₀₀ of 0.05. Expression was induced by the addition of 2% (w/v) galactose. The cultures were incubated for a further 96 hours at 20° C.

### Example 6

### Cloning of Expression Plasmids for the Seed-Specific Expression in Plants

To transform plants, a further transformation vector based on pSUN-USP was generated. To this end, NotI cleavage sites were inserted at the 5′ and 3′ end of the coding sequences, using PCR. The corresponding primer sequences were derived from the 5′ and 3 regions of OtElo1 and OtElo2.

Composition of the PCR mix (50 μl):

5.00 μl template cDNA

5.00 μl 10× buffer (Advantage polymerase)+25 mM MgCl₂

5.00 μl 2 mM dNTP

1.25 μl of each primer (10 μmol/μl)

0.50 μl Advantage polymerase

The Advantage polymerase from Clontech was employed.

PCR Reaction Conditions:

Annealing temperature: 1 min 55° C.

Denaturation temperature: 1 min 94° C.

Elongation temperature: 2 min 72° C.

Number of cycles: 35

The PCR products were incubated with the restriction enzyme NotI for 16 hours at 37° C. The plant expression vector pSUN300-USP was incubated in the same manner. Thereafter, the PCR products and the vector were separated by agarose gel electrophoresis and the corresponding DNA fragments were excised. The DNA was purified by means of the Qiagen Gel Purification Kit following the manufacturer's instructions. Thereafter, vector and PCR products were ligated. The Rapid Ligation Kit from Roche was used for this purpose. The resulting plasmids pSUN-OtELO1 and pSUN-OtELO2 were verified by sequencing.

pSUN300 is a derivative of plasmid pPZP (Hajdukiewicz, P, Svab, Z, Maliga, P., (1994). The small versatile pPZP family of Agrobacterium binary vectors for plant transformation. Plant Mol Biol 25:989-994). PSUN-USP was derived from pSUN300, by inserting a USP promoter into pSUN300 in the form of an EcoRI fragment. The polyadenylation signal is that of the Ostreococcus gene from the A. tumefaciens Ti plasmid (ocs-Terminator, Genbank Accession V00088) (De Greve, H., Dhaese, P., Seurinck, J., Lemmers, M., Van Montagu, M. and Schell, J. Nucleotide sequence and transcript map of the Agrobacterium tumefaciens Ti plasmid-encoded octopine synthase gene J. Mol. Appl. Genet. 1 (6), 499-511 (1982). The USP promoter corresponds to nucleotides 1 to 684 (Genbank Accession X56240), where part of the noncoding region of the USP gene is present in the promoter. The promoter fragment which is 684 base pairs in size was amplified by a PCR reaction and standard methods with the aid of a synthesized primer and by means of a commercially available T7 standard primer (Stratagene). Primer sequence:

5′-GTCGACCCGCGGACTAGTGGGCCCTCTAGACCCGGGGGATCCGGATC
TGCTGGCTATGAA-3′).

The PCR fragment was recut with EcoRI/SalI and inserted into the vector pSUN300 with OCS terminator. This gave rise to the plasmid with the name pSUN-USP. The construct was used for the transformation of Arabidopsis thaliana, oilseed rape, tobacco and linseed.

### Example 7

### Expression of OtELO1 and OtELO2 in Yeasts

Yeasts which had been transformed with the plasmids pYES3, pYES3-OtELO1 and pYES3-OtELO2 as described in Example 5 were analyzed as follows:

The yeast cells from the main cultures were harvested by centrifugation (100×g, 5 min, 20° C.) and washed with 100 mM NaHCO₃, pH 8.0 to remove residual medium and fatty acids. Starting with the yeast cell sediments, fatty acid methyl esters (FAMEs) were prepared by acid methanolysis. To this end, the cell sediments were incubated for one hour at 80° C. together with 2 ml of 1 N methanolic sulfuric acid and 2% (v/v) of dimethoxypropane. The FAMEs were extracted twice with petroleum ether (PE). To remove nonderivatized fatty acids, the organic phases were washed in each case once with 2 ml of 100 mM NaHCO₃, pH 8.0 and 2 ml of distilled water. Thereafter, the PE phases were dried with Na₂SO₄, evaporated under argon and taken up in 100 μl of PE. The samples were separated on a DB-23 capillary column (30 m, 0.25 mm, 0.25 μm, Agilent) in a Hewlett-Packard 6850 gas chromatograph equipped with flame ionization detector. The conditions for the GLC analysis were as follows: the oven temperature was programmed from 50° C. to 250° C. with a rate of 5° C./min and finally 10 min at 250° C. (holding).

The signals were identified by comparing the retention times with corresponding fatty acid standards (Sigma). The methodology is described for example in Napier and Michaelson, 2001, Lipids. 36(8):761-766; Sayanova et al., 2001, Journal of Experimental Botany. 52(360):1581-1585, Sperling et al., 2001, Arch. Biochem. Biophys. 388(2):293-298 and Michaelson et al., 1998, FEBS Letters. 439(3):215-218.

### Example 8

### Functional Characterization of OtELO1 and OtELO2

The substrate specificity of OtElo1 was determined after expression and after feeding various fatty acids (Tab. 2). The substrates fed can be detected in large amounts in all of the transgenic yeasts. The transgenic yeasts revealed the synthesis of novel fatty acids, the products of the OtElo1 reaction. This means that the gene OtElo1 was expressed functionally.

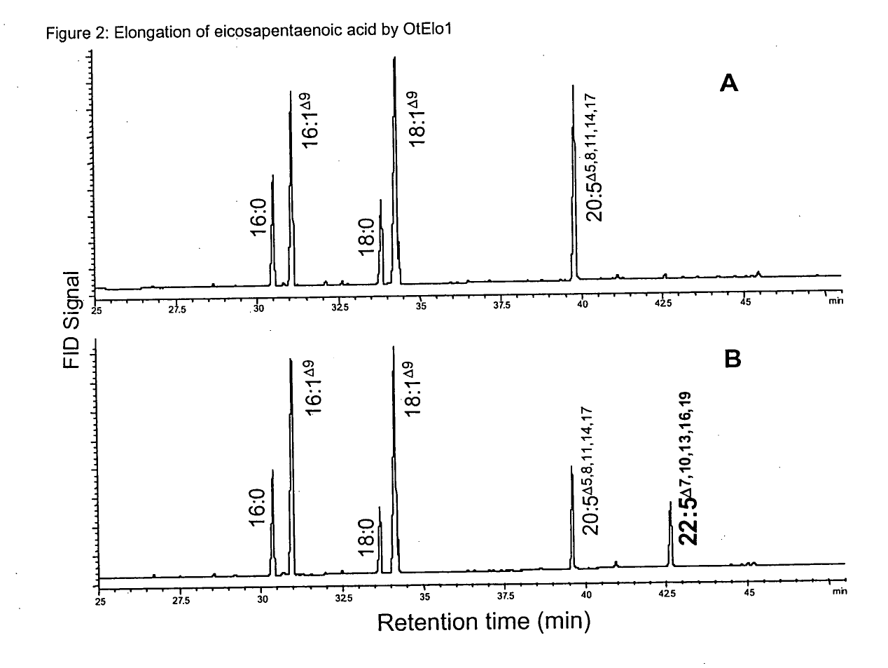

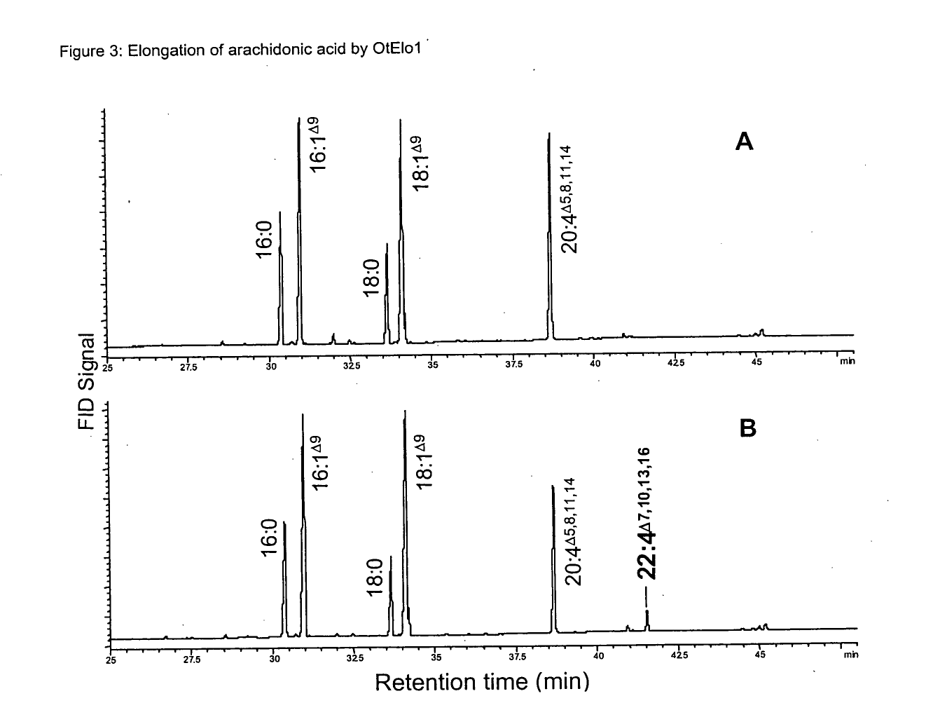

Table 2 shows that OtElo1 has a narrow degree of substrate specificity. OtElo1 was only capable of elongating the C₂₀-fatty acids eicosapentaenoic acid (FIG. 2) and arachidonic acid (FIG. 3), but preferentially eicosapentaenoic acid, which is ω3-desaturated.

TABLE 2

| Fatty acid substrate | Conversion rate (in %) |
|---|---|
| 16:0 | — |
| 16:1^{Δ9} | — |
| 18:0 | — |
| 18:1^{Δ9} | — |
| 18:1^{Δ11} | — |
| 18:2^{Δ9,12} | — |
| 18:3^{Δ6,9,12} | — |
| 18:3^{Δ5,9,12} | — |
| 20:3^{Δ8,11,14} | — |
| 20:4^{Δ5,8,11,14} | 10.8 ± 0.6 |
| 20:5^{Δ5,8,11,14,17} | 46.8 ± 3.6 |
| 22:4^{Δ7,10,13,16} | — |
| 22:6^{Δ4,7,10,13,16,19} | — |

Table 2 shows the substrate specificity of the elongase OtElo1 for C₂₀-polyunsaturated fatty acids with a double bond in the Δ5 position in comparison with various fatty acids.

The yeasts which had been transformed with the vector pOTE1 were grown in minimal medium in the presence of the fatty acids stated. The fatty acid methyl esters were synthesized by subjecting intact cells to acid methanolysis. Thereafter, the FAMEs were analyzed by GLC. Each value represents the mean (n=3)±standard deviation.

The substrate specificity of OtElo2 (SEQ ID NO: 1) was determined after expression and after feeding various fatty acids (Tab. 3). The substrates fed can be detected in large amounts in all of the transgenic yeasts. The transgenic yeasts revealed the synthesis of novel fatty acids, the products of the OtElo2 reaction. This means that the gene OtElo2 was expressed functionally.

TABLE 3

| Fatty acid substrate | Conversion rate (in %) |
|---|---|
| 16:0 | — |
| 16:1^{Δ9} | — |
| 16:3^{Δ7,10,13} | — |
| 18:0 | — |
| 18:1^{Δ6} | — |
| 18:1^{Δ9} | — |
| 18:1^{Δ11} | — |
| 18:2^{Δ9,12} | — |
| 18:3^{Δ6,9,12} | 15.3± |
| 18.3^{Δ5,9,12} | — |
| 18:4^{Δ6,9,12,15} | 21.1± |
| 20:2^{Δ11,14} | — |
| 20:3^{Δ8,11,14} | — |
| 20:4^{Δ5,8,11,14} | — |
| 20:5^{Δ5,8,11,14,17} | — |
| 22:4^{Δ7,10,13,16} | — |
| 22:5^{Δ7,10,13,16,19} | — |
| 22:6^{Δ4,7,10,13,16,19} | — |

Table 3 shows the substrate specificity of the elongase OtElo2 with regard to various fatty acids.

The yeasts which had been transformed with the vector pOTE2 were grown in minimal medium in the presence of the fatty acids stated. The fatty acid methyl esters were synthesized by subjecting intact cells to acid methanolysis. Thereafter, the FAMEs were analyzed by GLC. Each value represents the mean (n=3)±standard deviation.

The enzymatic activity shown in Table 3 clearly demonstrates that OtElo2 is a Δ6-elongase.

### Example 9

### Reconstitution of the Synthesis of DHA in Yeast

The reconstitution of the biosynthesis of DHA (22:6 ω3) can carried out starting from EPA (20:5 ω3) or stearidonic acid (18:4 ω3) by coexpressing OtElo1 together with the Euglena gracilis Δ4-desaturase or the Phaeodactylum tricornutum Δ5-desaturase and the Euglena gracilis Δ4-desaturase. To this end, the expression vectors pYes2-EgD4 and pESCLeu-PtD5 were additionally constructed. The abovementioned yeast strain which is already transformed with pYes3-OtElo1, can then be transformed further with pYes2-EgD4, or simultaneously with pYes2-EgD4 and pESCLeu-PtD5. The transformed yeasts can be selected on complete minimal dropout tryptophan and uracil medium agar plates supplemented with 2% glucose in the case of the pYes3-OtElo/pYes2-EgD4 strain and complete minimal dropout tryptophan, uracil and leucine medium in the case of the pYes3-OtElo/pYes2-EgD4+pESCLeu-PtD5 strain. Expression is then induced by addition of 2% (w/v) galactose. The cultures are subsequently incubated for a further 120 hours at 15° C.

### Example 10

### Generation of Transgenic Plants

a) Generation of transgenic oilseed rape plants (modified method of Moloney et al., 1992, Plant Cell Reports, 8:238-242)

The binary vectors in Agrobacterium tumefaciens C58C1:pGV2260 or Escherichia coli (Deblaere et al, 1984, Nucl. Acids. Res. 13, 4777-4788) were used for generating transgenic oilseed rape plants. To transform oilseed rape plants (Var. Drakkar, NPZ Nordeutsche Pflanzenzucht, Hohenlieth, Germany), a 1:50 dilution of an overnight culture of a positively transformed agrobacterial colony in Murashige-Skoog medium (Murashige and Skoog 1962 Physiol. Plant. 15, 473) supplemented with 3% sucrose (3MS medium) is used. Petioles or hypocotyls of freshly germinated sterile oilseed rape plants (in each case approx. 1 cm²) are incubated with a 1:50 agrobacterial dilution for 5-10 minutes in a petri dish. This is followed by 3 days of coincubation in the dark at 25° C. on 3MS medium supplemented with 0.8% Bacto agar. The cultures are then grown for 3 days at 16 hours light/8 hours dark. The cultivation is then continued in a weekly rhythm on MS medium supplemented with 500 mg/l Claforan (cefotaxime sodium), 50 mg/l kanamycin, 20 μM benzylaminopurine (BAP), then supplemented with 1.6 g/l of glucose. Growing shoots are transferred to MS medium supplemented with 2% sucrose, 250 mg/l Claforan and 0.8% Bacto agar. If no roots have developed after three weeks, 2-indolebutyric acid is added to the medium as growth hormone for rooting.

Regenerated shoots are obtained on 2MS medium supplemented with kanamycin and Claforan; after rooting, they were transferred to compost and, after growing on for two weeks in a controlled-environment cabinet or in the greenhouse, allowed to flower, and mature seeds were harvested and analyzed by lipid analysis for elongase expression, such as Δ5-elongase or Δ6-elongase activity. In this manner, lines with elevated contents of polyunsaturated C₂₀- and C₂₂-fatty acids can be identified.

b) Generation of Transgenic Linseed Plants

Transgenic linseed plants can be generated for example by the method of Bell et al., 1999, In Vitro Cell. Dev. Biol.-Plant. 35(6):456-465 by means of particle bombardment. Agrobacteria-mediated transformations can be generated for example by the method of Mlynarova et al. (1994), Plant Cell Report 13: 282-285.

### Example 11

### Cloning Desaturase Genes from Ostreococcus tauri

The search for conserved regions in the protein sequences with the aid of conserved motifs (His boxes, Domergue et al. 2002, Eur. J. Biochem. 269, 4105-4113) allowed the identification of five sequences with corresponding motifs in an Ostreococcus tauri sequence database (genomic sequences). The sequences are the following:

| Name of | | Amino | |
|---|---|---|---|
| gene | SEQ ID | acids | Homology |
| OtD4 | SEQ ID NO:7 | 536 | Δ4-desaturase |
| OtD5.1 | SEQ ID NO:9 | 201 | Δ5-desaturase |
| OtD5.2 | SEQ ID NO:11 | 237 | Δ5-desaturase |
| OtD6.1 | SEQ ID NO:13 | 457 | Δ6-desaturase |
| OtFad2 | SEQ ID NO:15 | 361 | Δ12-desaturase |

The alignments for finding homologies of the individual genes were carried out using the tBLASTn algorithm (Altschul et al., J. Mol. Biol. 1990, 215: 403-410). Cloning was carried out as follows:

40 ml of an Ostreococcus tauri culture in the stationary phase were spun down and the pellet was resuspended in 100 μl of double-distilled water and stored at −20° C. The relevant genomic DNAs were amplified based on the PCR method. The corresponding primer pairs were selected in such a way that they contained the yeast consensus sequence for highly efficient translation (Kozak, Cell 1986, 44:283-292) next to the start codon. The amplification of the OtDes-DNAs was carried out using in each case 1 μl of defrosted cells, 200 μM dNTPs, 2.5 U Taq polymerase and 100 μmol of each primer in a total volume of 50 μl. The conditions for the PCR were as follows: first denaturation at 95° C. for 5 minutes, followed by 30 cycles at 94° C. for 30 seconds, 55° C. for 1 minute and 72° C. for 2 minutes, and a final elongation step at 72° C. for 10 minutes.

The following primers were employed for the PCR:

OtD6.1 Forward:
5′ggtaccacataatgtgcgtggagacggaaaataacg3′
OtD6.1 Reverse:
5′ctcgagttacgccgtctttccggagtgttggcc3′

A comparison of the amino acid sequence of Ot6.1 with sequences from databases (National Center for Biotechnology, NCBI) by means of BLAST (Altschul et al., J. Mol. Biol. 1990, 215: 403-410) revealed that the highest sequence similarity is with a Δ5-desaturase from Thraustochytrium sp. (Table 4, selection of desaturases). The program GAP of the abovementioned software was used for the comparison.

TABLE 4
List of fatty acid desaturases with the highest sequence homologies
with the Ostreococcus Δ6 desaturase

| | | Iden- | | |
|---|---|---|---|---|
| | Regio- | tity | Homology | Accession |
| Organism | specificity | (%) | (%) | number |
| Thraustochytrium sp. | Δ5 position | 36 | 53 | AF489588 |
| (marine protist) | | | | |
| Mortierella alpina | Δ6 position | 19 | 35 | AB070557 |
| (fungus) | | | | |
| Rhizopus oryzae (fungus) | Δ6 position | 19 | 36 | AY583316 |
| Caenorhabditis elegans | Δ6 position | 18 | 32 | AF031477 |
| (worm) | | | | |
| Amylomyces rouxii | Δ6 position | 19 | 35 | AY392409 |
| (fungus) | | | | |
| Danio rerio (zebra fish) | Δ5/Δ6 | 20 | 38 | AF309556 |
| | position | | | |
| Homo sapiens (man) | Δ6 position | 21 | 36 | AF134404 |
| Sparus aurata (fish) | Δ6 position | 20 | 38 | AY055749 |

### Example: 12

### Cloning of Expression Plasmids for Heterologous Expression in Yeasts

To characterize the function of the desaturase OtD6.1 (=Δ6-desaturase) from Ostreococcus tauri, the open reading frame of the DNA downstream of the galactose-inducible GALL promoter of pYES2.1/V5-His-TOPO (Invitrogen) was cloned, giving rise to the corresponding pYES2.1-OtD6.1 clone. In a similar manner, further Ostreococcus desaturase genes can be cloned.

The Saccharomyces cerevisiae strain 334 was transformed with the vector pYES2.1-OtD6.1 by electroporation (1500 V). A yeast which was transformed with the blank vector pYES2 was used as control. The transformed yeasts were selected on complete minimal dropout uracil medium (CMdum) agar plates supplemented with 2% glucose. After the selection, in each case three transformants were selected for the further functional expression.

To express the OtD6.1 desaturase, precultures consisting of in each case 5 ml of CMdum dropout uracil liquid medium supplemented with 2% (w/v) raffinose were initially inoculated with the selected transformants and incubated for 2 days at 30° C. and 200 rpm. Then, 5 ml of CMdum (dropout uracil) liquid medium supplemented with 2% raffinose and 300 μM various fatty acids were inoculated with the precultures to an OD₆₀₀ of 0.05. Expression was induced by the addition of 2% (w/v) galactose. The cultures were incubated for a further 96 hours at 20° C.

### Example: 13

### Cloning of Expression Plasmids for the Seed-Specific Expression in Plants

A further transformation vector based on pSUN-USP is generated for the transformation of plants. To this end, NotI cleavage sites are introduced at the 5′ and 3′ ends of the coding sequences, using PCR. The corresponding primer sequences are derived from the 5′ and 3′ regions of the desaturases.

Composition of the PCR Mix (50 μl):

5.00 μl template cDNA

5.00 μl 10× buffer (Advantage polymerase)+25 mM MgCl₂

5.00 μl 2 mM dNTP

1.25 μl of each primer (10 μmol/μl)

0.50 μl Advantage polymerase

The Advantage polymerase from Clontech was employed.

PCR Reaction Conditions:

Annealing temperature: 1 min 55° C.

Denaturation temperature: 1 min 94° C.

Elongation temperature: 2 min 72° C.

Number of cycles: 35

The PCR products are incubated with the restriction enzyme NotI for 16 hours at 37° C. The plant expression vector pSUN300-USP is incubated in the same manner. Thereafter, the PCR products and the vector are separated by agarose gel electrophoresis and the corresponding DNA fragments are excised. The DNA is purified by means of the Qiagen Gel Purification Kit following the manufacturer's instructions. Thereafter, vector and PCR products are ligated. The Rapid Ligation Kit from Roche was used for this purpose. The resulting plasmids are verified by sequencing.

pSUN300 is a derivative of plasmid pPZP (Hajdukiewicz, P, Svab, Z, Maliga, P., (1994) The small versatile pPZP family of Agrobacterium binary vectors for plant transformation. Plant Mol Biol 25:989-994). pSUN-USP was derived from pSUN300, by inserting a USP promoter into pSUN300 in the form of an EcoRI fragment. The polyadenylation signal is that of the Ostreococcus gene from the A. tumefaciens Ti plasmid (ocs-Terminator, Genbank Accession V00088) (De Greve, H., Dhaese, P., Seurinck, J., Lemmers, M., Van Montagu, M. and Schell, J. Nucleotide sequence and transcript map of the Agrobacterium tumefaciens Ti plasmid-encoded octopine synthase gene J. Mol. Appl. Genet. 1 (6), 499-511 (1982)). The USP promoter corresponds to nucleotides 1 to 684 (Genbank Accession X56240), where part of the noncoding region of the USP gene is present in the promoter. The promoter fragment which is 684 base pairs in size was amplified by a PCR reaction and standard methods with the aid of a synthesized primer and by means of a commercially available T7 standard primer (Stratagene).

(Primer sequence:
5′-GTCGACCCGCGGACTAGTGGGCCCTCTAGACCCGGGGGATCCGGATC
TGCTGGCTATGAA-3′).

The PCR fragment was recut with EcoRI/SalI and inserted into the vector pSUN300 with OCS terminator. This gave rise to the plasmid with the name pSUN-USP. The construct was used for the transformation of Arabidopsis thaliana, oilseed rape, tobacco and linseed.

### Example: 14

### Expression of OtDes6.1 in Yeasts

Yeasts which had been transformed with the plasmids pYES2 and pYES2-OtDes6.1 as described in Example 11 were analyzed as follows:

The yeast cells from the main cultures were harvested by centrifugation (100×g, 5 min, 20° C.) and washed with 100 mM NaHCO₃, pH 8.0 to remove residual medium and fatty acids. The yeast cell sediments were extracted for 4 hours using chloroform/methanol (1:1). The resulting organic phase was extracted with 0.45% NaCl, dried with Na₂SO₄ and evaporated in vacuo. Applying thin-layer chromatography (horizontal tank, chloroform:methanol:acetic acid 65:35:8), the lipid extract was separated further into the lipid classes phosphatidylcholine (PC), phosphatidylinosotol (PI), phosphatidylserine (PS), phsophatidylethanolamine (PE) and neutral lipids (NL). The various separated spots on the thin-layer plate were scraped off. For the gas-chromatographic analysis, fatty acid methyl esters (FAMEs) were prepared by acid methanolysis. To this end, the cell sediments were incubated for one hour at 80° C. together with 2 ml of 1 N methanolic sulfuric acid and 2% (v/v) dimethoxypropane. The FAMEs were extracted twice with petroleum ether (PE). To remove nonderivatized fatty acids, the organic phases were washed in each case once with 2 ml of 100 mM NaHCO₃, pH 8.0 and 2 ml of distilled water. Thereafter, the PE phases were dried with Na₂SO₄, evaporated under argon and taken up in 100 μl of PE. The samples were separated on a DB-23 capillary column (30 m, 0.25 mm, 0.25 μm, Agilent) in a Hewlett-Packard 6850 gas chromatograph equipped with flame ionization detector. The conditions for the GLC analysis were as follows: the oven temperature was programmed from 50° C. to 250° C. with a 5° C./min increment and finally 10 min at 250° C. (holding).

The signals were identified by comparing the retention times with corresponding fatty acid standards (Sigma). The methodology is described for example in Napier and Michaelson, 2001, Lipids. 36(8):761-766; Sayanova et al., 2001, Journal of Experimental Botany. 52(360):1581-1585, Sperling et al., 2001, Arch. Biochem. Biophys. 388(2):293-298 and Michaelson et al., 1998, FEBS Letters. 439(3):215-218.

For the extraction of acyl-CoA esters 4 ml of yeast culture (OD_{60D}=1.5) according to the method of Scherling et al. 1996, J. Biol. Chem. 271, 22514-22521 were used. The derivatization of the acyl-CoA esters and analysis thereof by HPLC was carried at as described in Larson, T R and Graham I A, 2001 Plant J. 25, 115-125.

### Example: 15

### Functional Characterization of Ostreococcus Desaturases

The substrate specificity of desaturases can be determined after expression in yeast (see Examples cloning of desaturase genes, yeast expression) by feeding, using various yeasts. Descriptions for the determination of the individual activities can be found in WO 93/11245 for Δ15-desaturases, WO 94/11516 for Δ12-desaturases, WO 93/06712, U.S. Pat. No. 5,614,393, WO 96/21022, WO 0021557 and WO 99/27111 for Δ6-desaturases, Qiu et al. 2001, J. Biol. Chem. 276, 31561-31566 for Δ4-desaturases, Hong et al. 2002, Lipids 37, 863-868 for Δ5-desaturases.

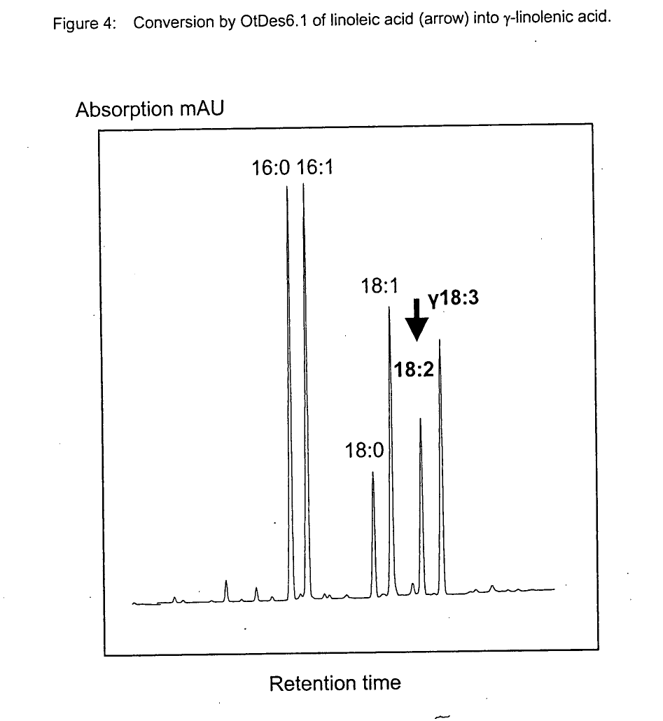

Table 4 shows the substrate specificity of the desaturase OtDes6.1 with regard to various fatty acids. The substrate specificity of OtDes6.1 was determined after expression and after feeding various fatty acids. The substrates fed can be detected in large amounts in all of the transgenic yeasts. The transgenic yeasts revealed the synthesis of novel fatty acids, the products of the OtDes6.2 reaction (FIG. 4). This means that the gene OtDes6.1 was expressed functionally.

The yeasts which had been transformed with the vector pYES2-OtDes6.1 were grown in minimal medium in the presence of the stated fatty acids. The fatty acid methyl esters were synthesized by subjecting intact cells to acid methanolysis. Thereafter, the FAMEs were analyzed via GLC. Each value represents the mean (n=3)±standard deviation. The activity corresponds to the conversion rate calculated using the formula [substrate/(substrate+product)*100].

Table 4 shows that OtDes6.1 has substrate specificity for linoleic and linolenic acid (18:2 and 18:3), since these fatty acids result in the highest activities. In contrast, the activity for oleic acid (18:1) and palmitoleic acid (16:1) is markedly less pronounced.

The preferred conversion of linoleic and linolenic acid demonstrates the suitability of this desaturase for the production of polyunsaturated fatty acids.

| Substrates | Activity in % |
|---|---|
| 16:1^{Δ9} | 5.6 |
| 18:1^{Δ9} | 13.1 |
| 18:2^{Δ9,12} | 68.7 |
| 18:3^{Δ9,12,15} | 64.6 |

FIG. 4 shows the conversion of linoleic acid by OtDes6.2. The FAMEs were analyzed via gas chromatography. The substrate fed (C18:2) is converted into γ-C18:3. Both starting material and product formed are indicated by arrows.

Kinetic analysis of the fatty acid shifts in acyl-CoA esters and lipids of yeast cultures which express OtDes6.1:

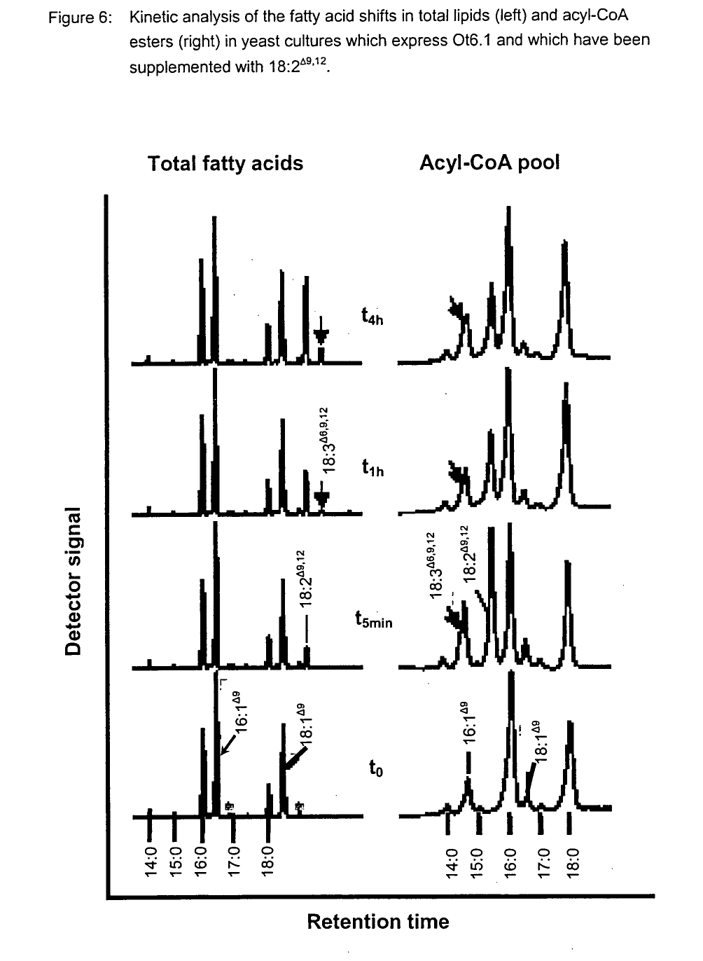

A culture of the yeast strain INVSc1, transformed with pYES-Ot6.1 (see Example 12), was incubated for 24 hours at 30° C. in the presence of galactose. Thereafter, 250 μM of linoleic acid (18:2Δ9,12) were added, and yeast cells were sampled and analyzed at different points in time (0 min, 5 min, 1 h, 4 h). The total fatty acid spectrum was analyzed by GC (FIG. 6, left) and the acyl-CoA esters by HPLC (FIG. 6, right).

It can be seen that the added fatty acid (18:2Δ9,12) can be detected both in the total lipids and in the acyl-CoA esters as early as at the first measurement (5 min). The product of the reaction of the Ot6.1 desaturase in the acyl-CoA esters can also be found at this early point in time. This product remains stable in terms of quantity over the remaining course of time. Only after 4 hours is the product clearly visible in the total lipids. The detection of the acyl-CoA ester product of the Ot6.1 desaturase before the product can be detected in the total lipids suggests that the desaturase utilizes CoA esters as the substrate and not phospholipids.

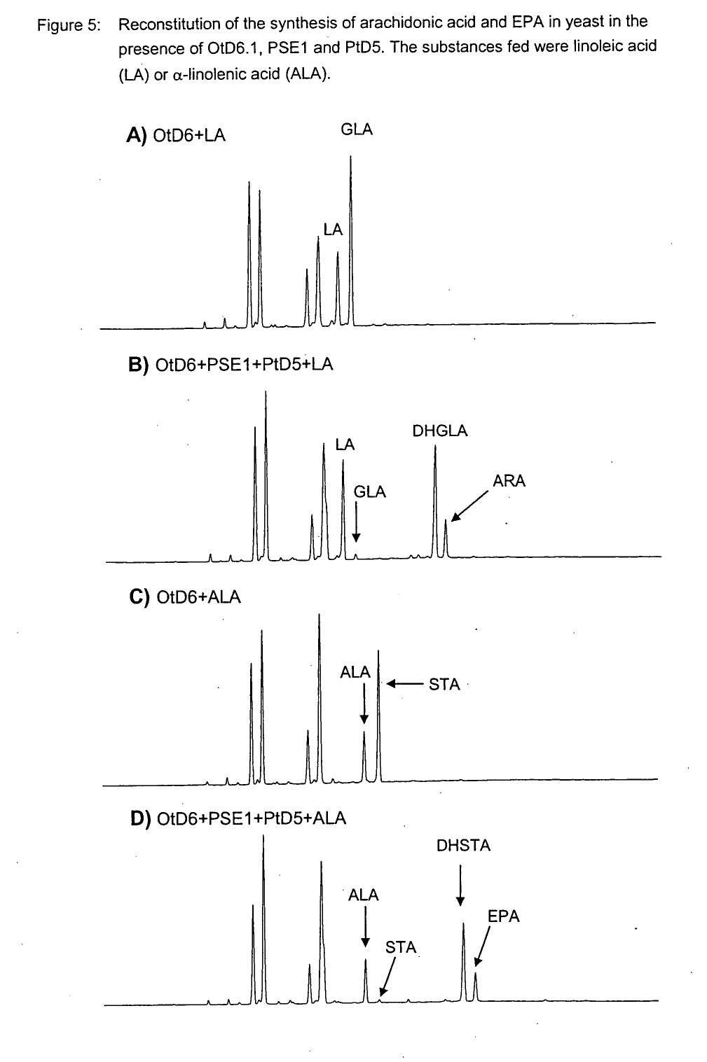

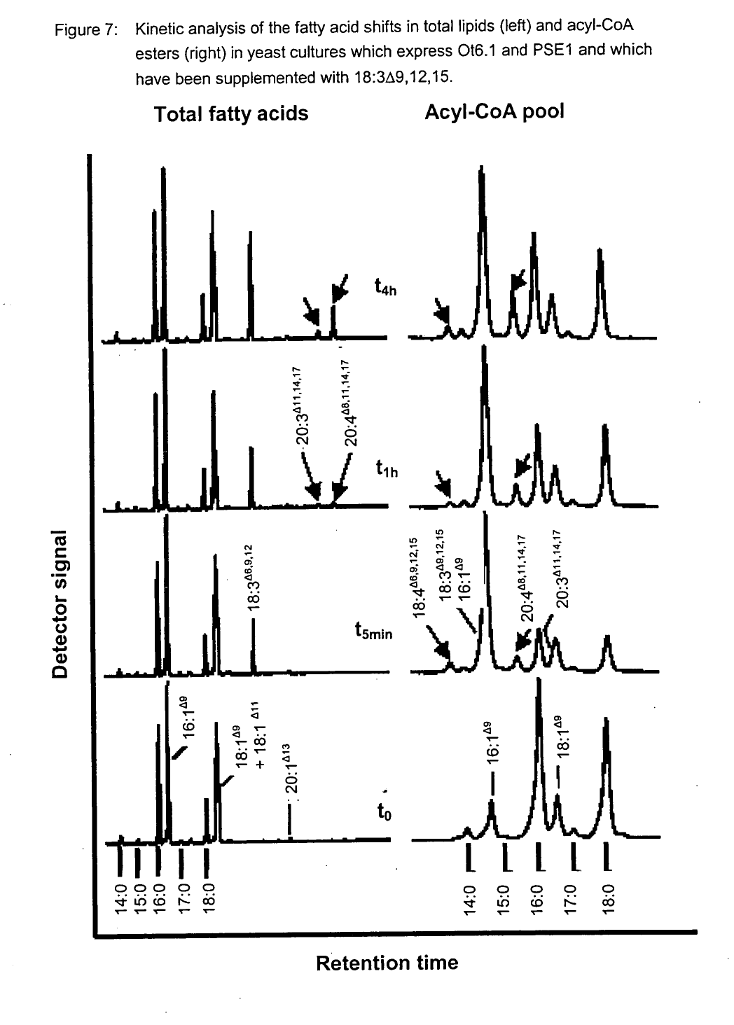

FIG. 7 shows the conversion of linoleic acid (=LA) and α-linolenic acid (=ALA) in the presence of OtDes6.1 to give γ-linolenic acid (=GLA) and stearidonic acid (=STA), respectively (FIGS. 5 A and C). Furthermore, FIG. 5 shows the conversion of linoleic acid (=LA) and α-linolenic acid (=ALA) in the presence of the Δ6-desaturase OtDes6.1 together with the Δ6-elongase PSE1 from Physcomitrella patens. (Zank et al. 2002, Plant J. 31:255-268) and the Δ5-desaturase PtD5 from Phaeodactylum tricornutum (Domergue et al. 2002, Eur. J. Biochem. 269, 4105-4113) to give dihomo-γ-linolenic acid (=DHGLA) and arachidonic acid (=ARA, FIG. 5 B) and to give dihomostearidonic acid (=DHSTA) and eicosapentaenoic acid (=EPA, FIG. 5 D), respectively. FIG. 5 shows clearly that the reaction products GLA and STA of the Δ6-desaturase OtDes6.1 in the presence of the Δ6-elongase PSE1 are elongated almost quantitatively to give DHGLA and DHSTA, respectively. The subsequent desaturation by the Δ5-desaturase PtD5 to give ARA and EPA, respectively, is also problem-free. Approximately 25-30% of the elongase product is desaturated (FIGS. 5 B and D). Table 5 gives an overview over the reconstitution of ARA and EPA, respectively. The parameters measured were the total fatty acids. In comparison with phospholipid-dependent desaturases as described, for example, in Domergue et al. 2002, Eur. J. Biochem. 269, 4105-4113, a clear increase (approx. 6-fold) of the end products ARA and EPA can be observed.

TABLE 5
Reconstitution of the PUFA biosynthesis in yeast.

| | 18:2^{Δ9,12}, | | 18:2^{Δ9,12,15}, | |
|---|---|---|---|---|
| | exogenously added | | exogenously added | |
| | | OtD6 + | | OtD6 + |
| | | PSE1 + | | PSE1 + |
| Fatty acid | Blank vector | PtD5 | Blank vector | PtD5 |
| 16:0 | 19.7 +/− 0.8 | 17.6 +/− 1.5 | 16.4 +/− 0.3 | 14.7 +/− 0.7 |
| 16:1^{Δ9} | 22.2 +/− 1.1 | 19.9 +/− 1.6 | 24.6 +/− 0.4 | 22.4 +/− 1.6 |
| 18:0 | 6.8 +/− 0.5 | 6.0 +/− 0.8 | 6.7 +/− 0.2 | 6.0 +/− 0.2 |
| 18:1 | 15.8 +/− 0.5 | 23.2 +/− 2.9 | 21.1 +/− 0.7 | 27.4 +/− 2.9 |
| 18:2^{Δ9,12} | 35.4 +/− 1.5 | 13.8 +/− 3.5 | — | — |
| 18:3^{Δ6,9,12} | — | 0.5 +/− 0.1 | — | — |
| 18:3^{Δ9,12,15} | — | — | 31.0 +/− 1.5 | 9.5 +/− 3.9 |
| 18:4^{Δ6,9,12,15} | — | — | — | 0.5 +/− 0.1 |
| 20:2^{Δ11,14} | — | 0.8 +/− 0.4 | — | — |
| 20:3^{Δ8,11,14} | — | 13.6 +/− 2.2 | — | — |
| 20:4^{Δ5,8,11,14} | — | 4.5 +/− 0.9 | — | — |
| 20:3^{Δ11,14,17} | — | — | — | 0.6 +/− 0.2 |
| 20:4^{Δ8,11,14,17} | — | — | — | 13.4 +/− 3.6 |
| 20:5^{Δ5,8,11,14,17} | — | — | — | 4.7 +/− 0.4 |

In a similar experiment as described hereinabove, the synthesis of isoARA (20:4Δ8,11,14,17) was also studied in the CoA ester pool. To this end, a yeast culture transformed with pYES-Ot6.1 and pLEU-PSE1 (described in Domergue et al. 2002, Eur. J. Biochem. 269, 4105-4113) was initiated and linolenic acid (18:3 Δ9,12,15) was added. After various points in time (0 min, 5 min, 1 h, 4 h) after the addition, yeast cells were sampled, and the total lipids were analyzed by GC (FIG. 7, left), while the acyl-CoA esters were analyzed by HPLC (FIG. 7, right).

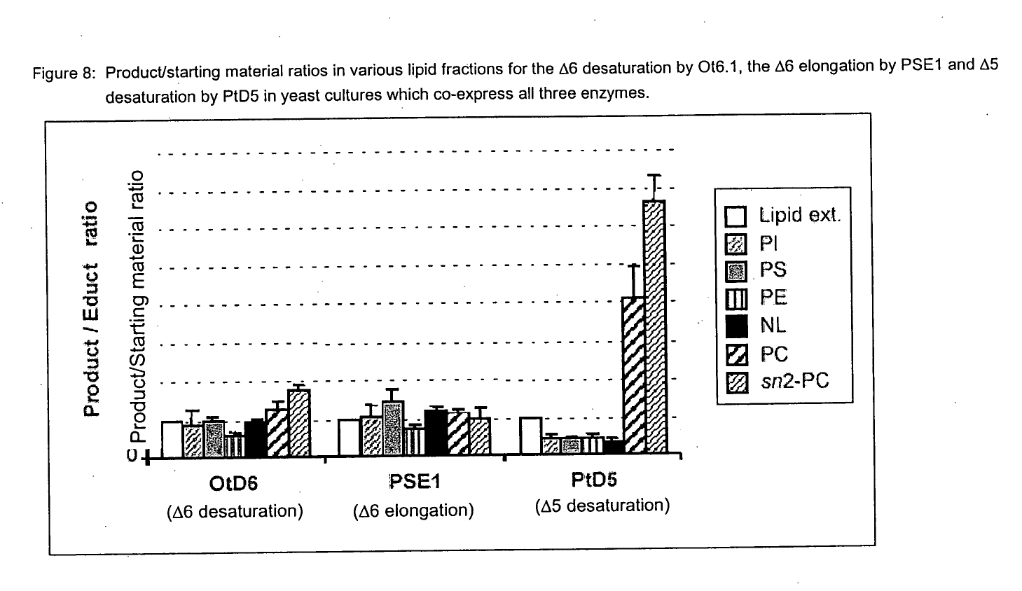

It was possible to demonstrate that both the Ot6.1 products and the products of the elongase PSE1 can be found at the earliest point in time in the acyl-CoA pool. Since the acyl-CoA esters act as substrate for elongases (Zank et al. 2002, Plant J, 31:255-268), this demonstrates that the Ot6.1 desaturase must also have the same substrate. FIG. 8 compiles this result. While the distribution of the two acyl-CoA-dependent enzymes OtD6 and PSE1 over the various lipid classes and positions is very homogeneous, this is not the case for the phospholipid-dependent Δ5-desaturase from Phaeodactylum tricornutum. Here, an accumulation of phosphatidylcholine in the sn-2 position can be demonstrated. As described in Domergue et al. 2002, Eur. J. Biochem. 269, 4105-4113, the Δ5-desaturase has phosphatidylcholine as its substrate.

Table 6 hereinbelow gives an overview of Ostreococcus desaturases which have been cloned:

Ostreococcus tauri desaturases

| Name | bp | aa | Homology | Cyt. B5 | His box1 | His box2 | His box3 |
|---|---|---|---|---|---|---|---|
| OtD4 | 1611 | 536 | Δ4-desaturase | HPGG | HCANH | WRYHHQVSHH | QVEHHLFP |
| OtD5.1 | 606 | 201 | Δ5-desaturase | — | — | — | QVVHHLFP |
| OtD5.2 | 714 | 237 | Δ5-desaturase | — | — | WRYHHMVSHH | QIEHHLPF |
| OtD6.1 | 1443 | 457 | Δ6-desaturase | HPGG | HEGGH | WNSMHNKHH | QVIHHLFP |
| OtFAD2 | 1086 | 361 | Δ12-desaturase | — | HECGH | WQRSHAVHH | HVAHH |

### Example 16

### Cloning of Expression Plasmids for the Seed-Specific Expression in Plants

A further transformation vector based on pSUN-USP is generated for the transformation of plants. To this end, NotI cleavage sites are introduced at the 5′ and 3′ ends of the coding sequences, using PCR. The corresponding primer sequences are derived from the 5′ and 3 regions of the desaturases.

Composition of the PCR Mix (50 μl):

5.00 μl template cDNA

5.00 μl 10× buffer (Advantage polymerase)+25 mM MgCl₂

5.00 μl 2 mM dNTP

1.25 μl of each primer (10 pmol/μl)

0.50 μl Advantage polymerase

The Advantage polymerase from Clontech was employed.

PCR Reaction Conditions:

Annealing temperature: 1 min 55° C.

Denaturation temperature: 1 min 94° C.

Elongation temperature: 2 min 72° C.

Number of cycles: 35

The PCR products are incubated with the restriction enzyme NotI for 16 hours at 37° C. The plant expression vector pSUN300-USP is incubated in the same manner. Thereafter, the PCR products and the vector are separated by agarose gel electrophoresis and the corresponding DNA fragments are excised. The DNA is purified by means of the Qiagen Gel Purification Kit following the manufacturer's instructions. Thereafter, vector and PCR products are ligated. The Rapid Ligation Kit from Roche was used for this purpose. The resulting plasmids are verified by sequencing.

pSUN300 is a derivative of plasmid pPZP (Hajdukiewicz, P, Svab, Z, Maliga, P., (1994) The small versatile pPZP family of Agrobacterium binary vectors for plant transformation. Plant Mol Biol 25:989-994). pSUN-USP originated from pSUN300, by inserting a USP promoter into pSUN300 in the form of an EcoRI fragment. The polyadenylation signal is the OCS gene from the A. tumefaciens Ti plasmid (ocs-Terminator, Genbank Accession V00088) (De Greve, H., Dhaese, P., Seurinck, J., Lemmers, M., Van Montagu, M. and Schell, J. Nucleotide sequence and transcript map of the Agrobacterium tumefaciens Ti plasmid-encoded octopine synthase gene J. Mol. Appl. Genet. 1 (6), 499-511 (1982)). The USP promoter corresponds to nucleotides 1 to 684 (Genbank Accession X56240), where part of the noncoding region of the USP gene is present in the promoter. The promoter fragment which is 684 base pairs in size was amplified by a PCR reaction and standard methods with the aid of a synthesized primer and by means of a commercially available T7 standard primer (Stratagene).

(Primer sequence:
5′-GTCGACCCGCGGACTAGTGGGCCCTCTAGACCCGGGGGATCCGGATC
TGCTGGCTATGAA-3′).

The PCR fragment was recut with EcoRI/SalI and inserted into the vector pSUN300 with OCS terminator. This gave rise to the plasmid with the name pSUN-USP. The construct was used for the transformation of Arabidopsis thaliana, oilseed rape, tobacco and linseed.

### Example 17

### Expression of Ostreococcus Desaturases in Yeasts

Yeasts which are transformed with the plasmids pYES2 and pYES2-Ostreococcus desaturases as described in Example 11 are analyzed as follows:

The yeast cells from the main cultures are harvested by centrifugation (100×g, 5 min, 20° C.) and washed with 100 mM NaHCO₃, pH 8.0 to remove residual medium and fatty acids. Starting with the yeast cell sediments, fatty acid methyl esters (FAMEs) are prepared by acid methanolysis. To this end, the cell sediments are incubated for one hour at 80° C. together with 2 ml of 1 N methanolic sulfuric acid and 2% (v/v) dimethoxypropane. The FAMEs were extracted twice with petroleum ether (PE). To remove nonderivatized fatty acids, the organic phases are washed in each case once with 2 ml of 100 mM NaHCO₃, pH 8.0 and 2 ml of distilled water. Thereafter, the PE phases are dried with Na₂SO₄, evaporated under argon and taken up in 100 μl of PE. The samples are separated on a DB-23 capillary column (30 m, 0.25 mm, 0.25 μm, Agilent) in a Hewlett-Packard 6850 gas chromatograph equipped with flame ionization detector. The conditions for the GLC analysis are as follows: the oven temperature is programmed from 50° C. to 250° C. with a 5° C./min increment and finally 10 min at 250° C. (holding).

The signals are identified by comparing the retention times with corresponding fatty acid standards (Sigma). The methodology is described for example in Napier and Michaelson, 2001, Lipids. 36(8):761-766; Sayanova et al., 2001, Journal of Experimental Botany. 52(360):1581-1585, Sperling et al., 2001, Arch. Biochem. Biophys. 388(2):293-298 and Michaelson et al., 1998, FEBS Letters. 439(3):215-218.

### Example 18

### Functional Characterization of Ostreococcus tauri Desaturases

The substrate specificity of desaturases can be determined after expression in yeast (see Examples cloning of desaturase genes, yeast expression) by feeding, using various yeasts. Descriptions for the determination of the individual activities can be found in WO 93/11245 for Δ15-desaturases, WO 94/11516 for Δ12-desaturases, WO 93/06712, U.S. Pat. No. 5,614,393, WO 96/21022, WO 0021557 and WO 99/27111 for Δ6-desaturases, Qiu et al. 2001, J. Biol. Chem. 276, 31561-31566 for Δ4-desaturases, Hong et al. 2002, Lipids 37, 863-868 for Δ5-desaturases. The activity of the individual desaturases is calculated from the conversion rate using the formula [substrate/(substrate+product)*100].

### EQUIVALENTS

Many equivalents of the specific embodiments according to the invention described herein can be identified or found by the skilled worker resorting simply to routine experiments. These equivalents are intended to be within the scope of the patent claims.

1. A process for the production of compounds of the formula I 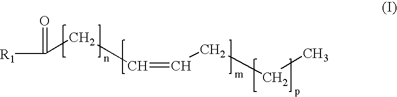
in a transgenic organism with a content of at least 1% by weight of the compounds based on the total lipid content of the transgenic organism, wherein the process comprises the following process steps:
a) introducing, into the organism, at least one nucleic acid sequence which encodes a polypeptide having Δ6-desaturase activity,
b) introducing, into the organism, at least one nucleic acid sequence which encodes a polypeptide having Δ6-elongase activity,
c) introducing, into the organism, at least one nucleic acid sequence which encodes a polypeptide having Δ5-desaturase activity,
d) introducing, into the organism, at least one nucleic acid sequence which encodes a polypeptide having Δ5-elongase activity, and
e) introducing, into the organism, at least one nucleic acid sequence which encodes a polypeptide having Δ4-desaturase activity,
wherein the variables and substituents in formula I have the following meanings:
R¹=hydroxyl, coenzyme A (thioester), lysophosphatidylcholine, lysophosphatidylethanolamine, lysophosphatidylglycerol, lysodiphosphatidylglycerol, lysophosphatidylserine, lysophosphatidylinositol, sphingo base or a radical of the formula II 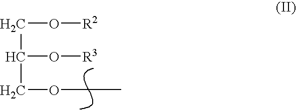
R²=hydrogen, lysophosphatidylcholine, lysophosphatidylethanolamine, lysophosphatidylglycerol, lysodiphosphatidylglycerol, lysophosphatidylserine, lysophosphatidylinositol or saturated or unsaturated C₂-C₂₄-alkylcarbonyl,
R³=hydrogen, saturated or unsaturated C₂-C₂₄-alkylcarbonyl, or R² and R³ independently of one another are a radical of the formula Ia: 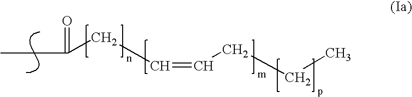
n=2, 3, 4, 5, 6, 7 or 9, m=2, 3, 4, 5 or 6 and p=0 or 3.

2. The process according to claim 1, wherein the nucleic acid sequences which encode polypeptides with Δ6-desaturase, Δ6-elongase, Δ5-desaturase, Δ5-elongase or Δ4-desaturase activity are selected from the group consisting of:
a) a nucleic acid sequence with the sequence shown in SEQ ID NO: 1, SEQ ID NO: 3, SEQ ID NO:5, SEQ ID NO: 7, SEQ ID NO: 9, SEQ ID NO: 11 or SEQ ID NO: 13,
b) nucleic acid sequences which, as the result of the degeneracy of the genetic code, are derived from the amino acid sequence shown in SEQ ID NO: 2, SEQ ID NO: 4, SEQ ID NO:6, SEQ ID NO: 8, SEQ ID NO: 10, SEQ ID NO: 12 or SEQ ID NO: 14, and
c) derivatives of the nucleic acid sequence shown in SEQ ID NO: 1, SEQ ID NO: 3, SEQ ID NO:5, SEQ ID NO: 7, SEQ ID NO: 9, SEQ ID NO: 11 or SEQ ID NO: 13 which encode polypeptides with at least 40% identity at the amino acid level with the sequence shown in SEQ ID NO: 2, SEQ ID NO: 4, SEQ ID NO:6, SEQ ID NO: 8, SEQ ID NO: 10, SEQ ID NO: 12 or SEQ ID NO: 14 and having Δ6-desaturase, Δ6-elongase, Δ5-desaturase, Δ5-elongase or Δ4-desaturase activity.

3. The process according to claim 1, wherein a nucleic acid sequence which encodes a polypeptide with Δ12-desaturase activity selected from the group consisting of:
a) a nucleic acid sequence with the sequence shown in SEQ ID NO: 15,
b) nucleic acid sequences which, as the result of the degeneracy of the genetic code, are derived from the amino acid sequence shown in SEQ ID NO: 16, and
c) derivatives of the nucleic acid sequence shown in SEQ ID NO:15 which encode polypeptides with at least 50% identity at the amino acid level with the sequence shown in SEQ ID NO: 16 and having Δ12-desaturase activity is additionally introduced into the organism.

4. The process according to claim 1, wherein the substituents R² or R³ independently of one another are saturated or unsaturated C₁₈-C₂₂-alkylcarbonyl.

5. The process according to claim 1, wherein the substituents R² or R³ independently of one another are unsaturated C₁₈-, C₂₀- or C₂₂-alkylcarbonyl with at least two double bonds.

6. The process according to claim 1, wherein the transgenic organism is a transgenic microorganism or a transgenic plant.

7. The process according to claim 1, wherein the transgenic organism is an oil-producing plant, a vegetable plant or an ornamental.

8. The process according to claim 1, wherein the transgenic organism is a transgenic plant selected from the group of the plant families: Adelotheciaceae, Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Crypthecodiniaceae, Cucurbitaceae, Ditrichaceae, Elaeagnaceae, Ericaceae, Euphorbiaceae, Fabaceae, Geraniaceae, Gramineae, Juglandaceae, Lauraceae, Leguminosae, Linaceae or Prasinophyceae.

9. The process according to claim 1, wherein the compounds of the formula I are isolated from the organism in the form of their oils, lipids or free fatty acids.

10. The process according to claim 1, wherein the compounds of the formula I are isolated in a concentration of at least 5% by weight based on the total lipid content of the transgenic organism.

11. An oil, lipid or fatty acid, or a fraction thereof, produced by the process according to claim 1.

12. An oil, lipid or fatty acid composition which comprises polyunsaturated fatty acids (PUFAs) produced by the process according to claim 1 from transgenic plants.

13. A process for the production of oils, lipids or fatty acid compositions by mixing oil, lipids or fatty acids produced by the process of claim 1 or oil, lipid or fatty acid compositions comprising PUFAs produced by the process of claim 1 from transgenic plants with animal oils, lipids or fatty acids.

14. A process for the preparation of feed, foodstuffs, cosmetics or pharmaceuticals, comprising utilizing the oil, lipids or fatty acids produced according to claim 1.

15. An isolated nucleic acid encoding a polypeptide with Δ6-desaturase activity, comprising a nucleotide sequence selected from the group consisting of:
a) a nucleic acid sequence with the sequence shown in SEQ ID NO: 13,
b) nucleic acid sequences which, as the result of the degeneracy of the genetic code, are derived from the amino acid sequence shown in SEQ ID NO:14, and
c) derivatives of the nucleic acid sequence shown in SEQ ID NO:13 which encode polypeptides with at least 40% homology at the amino acid level with the sequence shown in SEQ ID NO: 14 and having Δ6-desaturase activity.

16. An isolated nucleic acid encoding a polypeptide with Δ5-desaturase activity, comprising a nucleotide sequence selected from the group consisting of:
a) a nucleic acid sequence with the sequence shown in SEQ ID NO:9 or SEQ ID NO:11,
b) nucleic acid sequences which, as the result of the degeneracy of the genetic code, are derived from the amino acid sequence shown in SEQ ID NO:10 or SEQ ID NO:12, and
c) derivatives of the nucleic acid sequence shown in SEQ ID NO:9 or in SEQ ID NO: 11 which encode polypeptides with at least 40% homology at the amino acid level with the sequence shown in SEQ ID NO: 10 or in SEQ ID NO: 12 and having Δ5-desaturase activity.

17. An isolated nucleic acid encoding a polypeptide with Δ4-desaturase activity, comprising a nucleotide sequence selected from the group consisting of:
a) a nucleic acid sequence with the sequence shown in SEQ ID NO:7,
b) nucleic acid sequences which, as the result of the degeneracy of the genetic code, are derived from the amino acid sequence shown in SEQ ID NO:8, and
c) derivatives of the nucleic acid sequence shown in SEQ ID NO:7 which encode polypeptides with at least 40% homology at the amino acid level with the sequence shown in SEQ ID NO:8 and having Δ4-desaturase activity.

18. An isolated nucleic acid encoding a polypeptide with Δ12-desaturase activity, comprising a nucleotide sequence selected from the group consisting of:
a) a nucleic acid sequence with the sequence shown in SEQ ID NO: 15,
b) nucleic acid sequences which, as the result of the degeneracy of the genetic code, are derived from the amino acid sequence shown in SEQ ID NO:16, and
c) derivatives of the nucleic acid sequence shown in SEQ ID NO: 15 which encode polypeptides with at least 50% identity at the amino acid level with the sequence shown in SEQ ID NO:16 and having Δ12-desaturase activity.

19. The isolated nucleic acid according to claim 15, wherein the nucleic acid is derived from an alga, a fungus, a microorganism, a plant or a nonhuman animal.

20. The isolated nucleic acid according to claim 15, wherein the nucleic acid is derived from the order Salmoniformes, the diatom genera Thalassiosira or Crypthecodinium or from the family of the Prasinophyceae or Pythiaceae.

21. A polypeptide comprising an amino acid sequence which is encoded by the isolated nucleic acid according to claim 15.

22. A gene construct comprising the isolated nucleic acid according claim 15, wherein the nucleic acid is linked operably with one or more regulatory signals.

23. The gene construct according to claim 22, wherein the nucleic acid construct comprises additional biosynthesis genes of the fatty acid or lipid metabolism selected from the group acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene oxide synthases, hydroperoxide lyases or fatty acid elongase(s).

24. The gene construct according to claim 22, wherein the gene construct comprises additional biosynthesis genes of the fatty acid or lipid metabolism selected from the group consisting of Δ4-desaturase, Δ5-desaturase, Δ6-desaturase, Δ9-desaturase, Δ12-desaturase and Δ6-elongase.

25. A vector comprising a the nucleic acid according to claim 15.

26. A transgenic nonhuman organism, comprising at least one nucleic acid according to claim 15.

27. The transgenic nonhuman organism according to claim 26, wherein the organism is a microorganism, a nonhuman animal or a plant.

28. The transgenic nonhuman organism according to claim 26, wherein the organism is a plant.


SEQUENCE LISTING (19 sequences)

SEQ ID NO: 1 - DNA, 903 bp, Ostreococcus tauri
  CDS (1)..(903): Delta-5-Elongase
atgagcgcctccggtgcgctgctgcccgcgatcgcgttcgccgcgtacgcgtacgcgacgtacgcctacgcctttgagtggtcgcacgcgaatggcatcgacaacgtcgacgcgcgcgagtggatcggtgcgctgtcgttgaggctcccggcgatcgcgacgacgatgtacctgttgttctgcctggtcggaccgaggttgatggcgaagcgcgaggcgttcgacccgaaggggttcatgctggcgtacaatgcgtatcagacggcgttcaacgtcgtcgtgctcgggatgttcgcgcgagagatctcggggctggggcagcccgtgtgggggtcaaccatgccgtggagcgatagaaaatcgtttaagatcctcctcggggtgtggttgcactacaacaaccaatatttggagctattggacactgtgttcatggttgcgcgcaagaagacgaagcagttgagcttcttgcacgtttatcatcacgccctgttgatctgggcgtggtggttggtgtgtcacttgatggccacgaacgattgtatcgatgcctacttcggcgcggcgtgcaactcgttcattcacatcgtgatgtactcgtattatctcatgtcggcgctcggcattcgatgcccgtggaagcgatacatcacccaggctcaaatgctccaattcgtcattgtcttcgcgcacgccgtgttcgtgctgcgtcagaagcactgcccggtcacccttccttgggcgcaaatgttcgtcatgacgaacatgctcgtgctcttcgggaacttctacctcaaggcgtactcgaacaagtcgcgcggcgacggcgcgagttccgtgaaaccagccgagaccacgcgcgcgcccagcgtgcgacgcacgcgatctcgaaaaattgactaa

SEQ ID NO: 2 - protein, 300 aa, Ostreococcus tauri
MSASGALLPAIAFAAYAYATYAYAFEWSHANGIDNVDAREWIGALSLRLPAIATTMYLLFCLVGPRLMAKREAFDPKGFMLAYNAYQTAFNVVVLGMFAREISGLGQPVWGSTMPWSDRKSFKILLGVWLHYNNQYLELLDTVFMVARKKTKQLSFLHVYHHALLIWAWWLVCHLMATNDCIDAYFGAACNSFIHIVMYSYYLMSALGIRCPWKRYITQAQMLQFVIVFAHAVFVLRQKHCPVTLPWAQMFVMTNMLVLFGNFYLKAYSNKSRGDGASSVKPAETTRAPSVRRTRSRKID

SEQ ID NO: 3 - DNA, 879 bp, Ostreococcus tauri
  CDS (1)..(879): Delta-6-Elongase
atgagtggcttacgtgcacccaactttttacacagattctggacaaagtgggactacgcgatttccaaagtcgtcttcacgtgtgccgacagttttcagtgggacatcgggccagtgagttcgagtacggcgcatttacccgccattgaatcccctaccccactggtgactagcctcttgttctacttagtcacagttttcttgtggtatggtcgtttaaccaggagttcagacaagaaaattagagagcctacgtggttaagaagattcataatatgtcataatgcgttcttgatagtcctcagtctttacatgtgccttggttgtgtggcccaagcgtatcagaatggatatactttatggggtaatgaattcaaggccacggaaactcagcttgctctctacatttacattttttacgtaagtaaaatatacgagtttgtagatacttacattatgcttctcaagaataacttgcggcaagtaagtttcctacacatttatcaccacagcacgatttcctttatttggtggatcattgctcggagggctccgggtggtgatgcttacttcagcgcggccttgaactcatgggtacacgtgtgcatgtacacctattatctattatcaacccttattggaaaagaagatcctaagcgttccaactacctttggtggggtcgccacctaacgcaaatgcagatgcttcagtttttcttcaacgtacttcaagcgttgtactgcgcttcgttctctacgtatcccaagtttttgtccaaaattctgctcgtctatatgatgagccttctcggcttgtttgggcatttctactattccaagcacatagcagcagctaagctccagaaaaaacagcagtga

SEQ ID NO: 4 - protein, 292 aa, Ostreococcus tauri
MSGLRAPNFLHRFWTKWDYAISKVVFTCADSFQWDIGPVSSSTAHLPAIESPTPLVTSLLFYLVTVFLWYGRLTRSSDKKIREPTWLRRFIICHNAFLIVLSLYMCLGCVAQAYQNGYTLWGNEFKATETQLALYIYIFYVSKIYEFVDTYIMLLKNNLRQVSFLHIYHHSTISFIWWIIARRAPGGDAYFSAALNSWVHVCMYTYYLLSTLIGKEDPKRSNYLWWGRHLTQMQMLQFFFNVLQALYCASFSTYPKFLSKILLVYMMSLLGLFGHFYYSKHIAAAKLQKKQQ

SEQ ID NO: 5 - DNA, 879 bp, Ostreococcus tauri
  CDS (1)..(879): Delta-6-Elongase
atgagtggcttacgtgcacccaactttttacacagattctggacaaagtgggactacgcgatttccaaagtcgtcttcacgtgtgccgacagttttcagtgggacatcgggccagtgagttcgagtacggcgcatttacccgccattgaatcccctaccccactggtgactagcctcttgttctacttagtcacagttttcttgtggtatggtcgtttaaccaggagttcagacaagaaaattagagagcctacgtggttaagaagattcataatatgtcataatgcgttcttgatagtcctcagtctttacatgtgccttggttgtgtggcccaagcgtatcagaatggatatactttatggggtaatgaattcaaggccacggaaactcagcttgctctctacatttacattttttacgtaagtaaaatatacgagtttgtagatacttacattatgcttctcaagaataacttgcggcaagtaagattcctacacacttatcaccacagcacgatttcctttatttggtggatcattgctcggagggctccgggtggtgatgcttacttcagcgcggccttgaactcatgggtacacgtgtgcatgtacacctattatctattatcaacccttattggaaaagaagatcctaagcgttccaactacctttggtggggtcgccacctaacgcaaatgcagatgcttcagtttttcttcaacgtacttcaagcgttgtactgcgcttcgttctctacgtatcccaagtttttgtccaaaattctgctcgtctatatgatgagccttctcggcttgtttgggcatttctactattccaagcacatagcagcagctaagctccagaaaaaacagcagtga

SEQ ID NO: 6 - protein, 292 aa, Ostreococcus tauri
MSGLRAPNFLHRFWTKWDYAISKVVFTCADSFQWDIGPVSSSTAHLPAIESPTPLVTSLLFYLVTVFLWYGRLTRSSDKKIREPTWLRRFIICHNAFLIVLSLYMCLGCVAQAYQNGYTLWGNEFKATETQLALYIYIFYVSKIYEFVDTYIMLLKNNLRQVRFLHTYHHSTISFIWWIIARRAPGGDAYFSAALNSWVHVCMYTYYLLSTLIGKEDPKRSNYLWWGRHLTQMQMLQFFFNVLQALYCASFSTYPKFLSKILLVYMMSLLGLFGHFYYSKHIAAAKLQKKQQ

SEQ ID NO: 7 - DNA, 1611 bp, Ostreococcus tauri
  CDS (1)..(1611): Delta-4-Desaturase
atgtacctcggacgcggccgtctcgagagcgggacgacgcgagggatgatgcggacgcacgcgcggcgaccgtcgacgacgtcgaatccgtgcgcgcggtcacgcgtgcgtaagacgacggagcgatcgctcgcgcgagtgcgacgatcgacgagtgagaagggaagcgcgctcgtgctcgagcgagagagcgaacgggagaaggaggagggagggaaagcgcgagcggagggattgcgattccaacgcccggacgtcgccgcgccggggggagcggatccttggaacgacgagaagtggacaaagaccaagtggacggtattcagagacgtcgcgtacgatctcgatcctttcttcgctcgacaccccggaggagactggctcctgaacttggccgtgggacgagactgcaccgcgctcatcgaatcctatcacttgcgaccagaggtggcgacggctcgtttcagaatgctgcccaaactcgaggattttcccgtcgaggccgtgcccaagtccccgagaccgaacgattcgccgttatacaacaacattcgcaaccgagtccgcgaagagctcttcccagaggagggaaagaatatgcacagacagggcggcgaccacggcgacggtgacgattctgggtttcgccgccttttgcttatgccgtgtacctattcccttccgggggttcctttccggctgcctcctcgggtctcgcgggggcgtggattggtctcacgattcaggcactgcgccaaccacggcgcgatgtctccttcgccggccgttaacggcgtcctcggtttgacgaacgatctcatcggcggctcgtccttgatgtggagatatcaccaccaagtcagccaccacattcattgcaacgacaacgccatggatcaagacgtgtacacggcgatgccattattgcgtttcgacgctcgccggcccaagtcctggtaccatcgcttccagcagtggtacatgtttttagcgttcccgttgttgcaggttgccttccaagtcggagacattgccgcactgttcacgcgtgataccgaaggcgctaagcttcacggggcgacgacgtgggagcttaccacggttgtcctcggtaagattgtgcacttcggtcttttgttggggccgttgatgaaccacgcggtgagttctgttttgctggggatcgtcggtttcatggcgtgccaaggtatagttctggcgtgcacgtttgctgtgagtcacaatgtcgcggaggcgaagatacctgaggacaccggaggagaagcctgggagagagattggggtgtccagcagttggtgactagcgccgactggggtggaaagataggtaacttcttcacgggtggcctcaacttgcaagttgagcaccacttgtttccggcgatttgcttcgtccactacccggacatcgcgaagatcgtgaaggaagaagcggccaagctcaacatcccttacgcgtcttacaggactcttcctggtattttcgtccaattctggagatttatgaaggacatgggcacggctgagcaaattggtgaagttccattgccgaagattcccaacccgcagctcgcgccgaagctcgcttag

SEQ ID NO: 8 - protein, 536 aa, Ostreococcus tauri
MYLGRGRLESGTTRGMMRTHARRPSTTSNPCARSRVRKTTERSLARVRRSTSEKGSALVLERESEREKEEGGKARAEGLRFQRPDVAAPGGADPWNDEKWTKTKWTVFRDVAYDLDPFFARHPGGDWLLNLAVGRDCTALIESYHLRPEVATARFRMLPKLEDFPVEAVPKSPRPNDSPLYNNIRNRVREELFPEEGKNMHRQGGDHGDGDDSGFRRLLLMPCTYSLPGVPFRLPPRVSRGRGLVSRFRHCANHGAMSPSPAVNGVLGLTNDLIGGSSLMWRYHHQVSHHIHCNDNAMDQDVYTAMPLLRFDARRPKSWYHRFQQWYMFLAFPLLQVAFQVGDIAALFTRDTEGAKLHGATTWELTTVVLGKIVHFGLLLGPLMNHAVSSVLLGIVGFMACQGIVLACTFAVSHNVAEAKIPEDTGGEAWERDWGVQQLVTSADWGGKIGNFFTGGLNLQVEHHLFPAICFVHYPDIAKIVKEEAAKLNIPYASYRTLPGIFVQFWRFMKDMGTAEQIGEVPLPKIPNPQLAPKLA

SEQ ID NO: 9 - DNA, 606 bp, Ostreococcus tauri
  CDS (1)..(606): Delta-5-Desaturase
atgtacggtttgctatcgctcaagtcgtgcttcgtcgacgatttcaacgcctacttctccggacgcatcggctgggtcaaggtgatgaagttcacccgcggcgaggcgatcgcattttggggcaccaagctcttgtgggccgcgtattacctcgcgttgccgctaaagatgtcgcatcggccgctcggagaactcctcgcactctgggccgtcaccgagttcgtcaccggatggctgttggcgttcatgttccaagtcgcccacgtcgtcggcgaggttcacttcttcaccctcgacgcgaagaaccgcgtgaacttgggatggggagaggcacagctcatgtcgagcgcggatttcgcccacggatccaagttttggacgcacttctccggaggcttaaactaccaagtcgtccaccatctcttcccgggcgtctgccacgtgcactatcccgcgctcgcgccaattattaaggcggcagctgagaagcacggcctccactaccagatttaccccacgttttggtccgccctgcgcgcgcacttccggcacctcgccaacgtcggccgcgccgcgtacgtaccgtccctccaaaccgtcggatga

SEQ ID NO: 10 - protein, 201 aa, Ostreococcus tauri
MYGLLSLKSCFVDDFNAYFSGRIGWVKVMKFTRGEAIAFWGTKLLWAAYYLALPLKMSHRPLGELLALWAVTEFVTGWLLAFMFQVAHVVGEVHFFTLDAKNRVNLGWGEAQLMSSADFAHGSKFWTHFSGGLNYQVVHHLFPGVCHVHYPALAPIIKAAAEKHGLHYQIYPTFWSALRAHFRHLANVGRAAYVPSLQTVG

SEQ ID NO: 11 - DNA, 714 bp, Ostreococcus tauri
  CDS (1)..(714): Delta-5-Desaturase
atggtgagccatcactcgtactgtaacgacgcggatttggatcaggatgtgtacaccgcactgccgctcctgcgcctggacccgtctcaggagttgaagtggtttcatcgataccaggcgttttacgccccgctcatgtggccgtttttgtggctcgcggcgcagtttggcgacgcgcagaacatcctgatcgaccgagcgtcgccgggcgtcgcgtacaagggattgatggcgaacgaggtcgcgctgtacgttctcggtaaggttttacactttggtcttctcctcggcgttcctgcgtacttgcacggattgtccaacgcgatcgttccattcttggcgtacggcgcattcggctccttcgtcctgtgctggttcttcatcgtcagccataacctcgaagcgctgacacccgttaaccttaacaagtccacgaagaacgactggggggcgtggcagatcgagacatcggcgtcttggggcaacgcgttctggagcttcttctctggaggtctgaacctgcaaatcgagcaccacctcttcccgggcatggcgcacaacctgtacccgaagatggtgccgatcatcaaggacgagtgtgcgaaagcgggcgttcgctacaccggttacggtggctacaccggcctgctcccgatcacccgcgacatgttctcctacctccataagtgtggccgaacggcgaaactagcctaa

SEQ ID NO: 12 - protein, 237 aa, Ostreococcus tauri
MVSHHSYCNDADLDQDVYTALPLLRLDPSQELKWFHRYQAFYAPLMWPFLWLAAQFGDAQNILIDRASPGVAYKGLMANEVALYVLGKVLHFGLLLGVPAYLHGLSNAIVPFLAYGAFGSFVLCWFFIVSHNLEALTPVNLNKSTKNDWGAWQIETSASWGNAFWSFFSGGLNLQIEHHLFPGMAHNLYPKMVPIIKDECAKAGVRYTGYGGYTGLLPITRDMFSYLHKCGRTAKLA

SEQ ID NO: 13 - DNA, 1371 bp, Ostreococcus tauri
  CDS (1)..(1371): Delta-6-Desaturase
atgtgcgtggagacggaaaataacgatgggatccccacggtggagatcgcgttcgacggtgagcgcgagcgggcggaggcaaacgtgaagctgtccgcggagaagatggagccggcggcgctggcgaagacgttcgcgaggcggtacgtcgtgatcgagggggtggagtacgatgtgacggattttaagcacccgggaggaacggttattttctatgcgttgtcaaacaccggggcggacgcgacggaagcgttcaaggagtttcatcatcggtcgagaaaggcgaggaaagccttggcggcgctcccgtctcgaccggccaagacggccaaggtggacgacgcggagatgctccaagatttcgccaagtggcggaaagaattggagagagatggattcttcaagccctctccggcgcacgtggcgtatcgcttcgccgagctcgcggcgatgtacgctctcgggacgtacctgatgtacgctcgatacgtcgtctcctcggtgctcgtgtacgcttgctttttcggcgcccgatgcggttgggtgcagcacgagggcggacacagctcgctgacgggcaacatttggtgggacaagcgcatccaggccttcacagccgggttcggtctcgccggtagcggcgacatgtggaactcgatgcacaacaagcatcacgcgacgcctcaaaaggttcgtcacgacatggatctggacaccacccccgcggtggcgttcttcaacaccgcggtggaagacaatcgtccccgtggctttagcaagtactggttgcgccttcaggcgtggaccttcatccccgtgacgtccggcttggtgctccttttctggatgtttttcctccacccctccaaggctttgaagggtggcaagtacgaagagttggtgtggatgctcgccgcgcacgtcatccgcacgtggacgatcaaggcggtgaccggattcaccgcgatgcagtcctacggcttatttttggcgacgagctgggtgagcggctgctatctgtttgcacacttctccacgtcgcacacgcacctggatgtggtgcccgcggacgagcatctctcctgggttcgatacgccgtcgatcacacgatcgacatcgatccgagtcaaggttgggtgaactggttgatgggctacctcaactgccaagtcatccaccacctctttccgagcatgccgcagttccgccagcccgaggtatctcgccgcttcgtcgcctttgcgaaaaagtggaacctcaactacaaggtcatgacctacgccggtgcgtggaaggcaacgctcggaaacctcgacaacgtgggtaagcactactacgtgcacggccaacactccggaaagacggcgtaa

SEQ ID NO: 14 - protein, 456 aa, Ostreococcus tauri
MCVETENNDGIPTVEIAFDGERERAEANVKLSAEKMEPAALAKTFARRYVVIEGVEYDVTDFKHPGGTVIFYALSNTGADATEAFKEFHHRSRKARKALAALPSRPAKTAKVDDAEMLQDFAKWRKELERDGFFKPSPAHVAYRFAELAAMYALGTYLMYARYVVSSVLVYACFFGARCGWVQHEGGHSSLTGNIWWDKRIQAFTAGFGLAGSGDMWNSMHNKHHATPQKVRHDMDLDTTPAVAFFNTAVEDNRPRGFSKYWLRLQAWTFIPVTSGLVLLFWMFFLHPSKALKGGKYEELVWMLAAHVIRTWTIKAVTGFTAMQSYGLFLATSWVSGCYLFAHFSTSHTHLDVVPADEHLSWVRYAVDHTIDIDPSQGWVNWLMGYLNCQVIHHLFPSMPQFRQPEVSRRFVAFAKKWNLNYKVMTYAGAWKATLGNLDNVGKHYYVHGQHSGKTA

SEQ ID NO: 15 - DNA, 1086 bp, Ostreococcus tauri
  CDS (1)..(1086): Delta-12-Desaturase
atgcaggagggggtgcgaaacattccgaacgagtgctttgagacgggacatcttgaaagaccctggcgttccggccggtgtgggcgcgatcccggttcgaattggggcgctggcttccgctttttttcgctcaaggggttttggtggccggcgtggtgggcgtacgcgttcgtgacggggacggcggccactgggtgttgggtcgccgcgcacgagtgcgggcacggcgcgttcagcgataacaagacgttgcaagatgcggttggatacgtgttgcactcgttgctcttggtgccgtacttttcttggcagcgatcacacgcggtgcatcactcgaggacgaatcacgttcttgagggcgagacgcacgtgccggcgcgcttggggacggaagacgccaacgtcgtgttcaagcttcgcgaattgatcggtgaagggccgttcacgtttttcaacctcgtcggcgtcttcgcgctcggatggccgatttacttgctcaccggcgcgagcggcggaccggtgcgcggtaacacgaaccacttcttacccttcatgggcgagaaaggtaagcacgcgctgttcccgggtaagtgggcgaagaaggtgtggcagtctgacatcggcgttgttgccgtcctgggcgcgctcgcggcttgggcggcgcacagcgggattgccacagtgatggcactctacgtcggcccgtacatggtgaccaacttttggctcgtcttgtacacgtggttacagcacaccgacgttgacgtgccgcacttcgagggcgacgattggaacttggtcaagggggcattcatgacgatcgatcgcccgtacggcccagtttttgatttcttgcaccaccgcatcggcagcacgcacgtcgcgcaccacatcaacacaccattcccgcattacaaggctcaaatggcgacggatgcgctaaaggaggcgtatcccgacctctacctttacgatccaactccgatcgcgaccgctacgtggcgcgtggggagcaagtgcatcgccgtcgtgaagaagggagacgaatgggtgttcacggataagcaactcccggtcgcggcgtga

SEQ ID NO: 16 - protein, 361 aa, Ostreococcus tauri
MQEGVRNIPNECFETGHLERPWRSGRCGRDPGSNWGAGFRFFSLKGFWWPAWWAYAFVTGTAATGCWVAAHECGHGAFSDNKTLQDAVGYVLHSLLLVPYFSWQRSHAVHHSRTNHVLEGETHVPARLGTEDANVVFKLRELIGEGPFTFFNLVGVFALGWPIYLLTGASGGPVRGNTNHFLPFMGEKGKHALFPGKWAKKVWQSDIGVVAVLGALAAWAAHSGIATVMALYVGPYMVTNFWLVLYTWLQHTDVDVPHFEGDDWNLVKGAFMTIDRPYGPVFDFLHHRIGSTHVAHHINTPFPHYKAQMATDALKEAYPDLYLYDPTPIATATWRVGSKCIAVVKKGDEWVFTDKQLPVAA

SEQ ID NO: 17 - DNA, 60 bp, Artificial sequence
  Primer
gtcgacccgcggactagtgggccctctagacccgggggatccggatctgctggctatgaa

SEQ ID NO: 18 - DNA, 36 bp, Artificial sequence
  PCR primer
ggtaccacataatgtgcgtggagacggaaaataacg

SEQ ID NO: 19 - DNA, 33 bp, Artificial sequence
  PCR primer
ctcgagttacgccgtctttccggagtgttggcc